(19) United States
(12) Patent Application Publication
(10) Pub. No.: US 2026/0226179 A1
(43) Pub. Date: Aug. 6, 2026

(54) ANTIBODIES BINDING TO HUMAN INTERLEUKIN-6 RECEPTOR

(71) Applicant: MAB DESIGNS LIMITED, London, Greater London (GB)

(72) Inventors: Andrew CLARK, London, Greater London (GB); John WIJDENES, London, Greater London (GB)

(21) Appl. No.: 19/151,967

(22) PCT Filed: Feb. 13, 2024
(86) PCT No.: PCT/EP2024/053654

§ 371 Date: Jul. 29, 2025

(30) Foreign Application Priority Data
Feb. 13, 2023 (GB) 2302043.1

(51) Int. Cl. C07K 16/28; A61P 35/00; A61P 37/06

(52) U.S. Cl. CPC C07K 16/2866; A61P 35/00; A61P 37/06; C07K 2317/33; C07K 2317/40; C07K 2317/55; C07K 2317/565; C07K 2317/76

(57) ABSTRACT

The present disclosure provides an antibody (or antigen binding protein) comprising at least one, preferably two, antigen binding domains that binds to human IL-6 receptor, wherein said antibody inhibits IL-6 trans signalling through the human soluble IL-6 receptor while maintaining IL-6 classical signalling through the human membrane-bound IL-6 receptor, and wherein said antibody can bind to human soluble IL-6 receptor and human membrane-bound IL-6 receptor. Compositions comprising said antibodies or antigen binding proteins are also provided, together with encoding nucleic acid molecules and expression vectors.

This invention relates generally to the field of antibodies and binding proteins that bind to human Interleukin-6 receptor (IL-6R), in particular anti-IL-6R antibodies that inhibit IL-6-mediated trans signalling while maintaining (e.g. not affecting) IL-6-mediated classical signalling. Such anti-IL-6R antibodies or binding proteins have therapeutic uses, such as treatment of inflammatory diseases and cancer. Binding protein and antibody-based compositions, methods and kits are also provided.

Interleukin-6 (IL-6) is a pleiotropic cytokine which functions as both a pro-inflammatory and an immune-modulatory or anti-inflammatory mediator dependent upon the signalling modality (Rose-John et al., Cytokine 144, 155577, 2021). It has a significant role in both innate and acquired immunity, in the maintenance and regeneration of neural cells and as a myokine in the regulation of metabolism (Rose-John et al., supra). IL-6 is also a prominent growth factor of tumour cells and plays a major role in inflammatory diseases and cancer (Rose-John et al., supra).

Interleukin-6 receptor (IL-6R) functions as a component of the signalling complex for IL-6. IL-6R exists in two forms: a membrane-bound form (membrane-bound IL-6R) which is expressed on the cell membrane of a limited number of cells (e.g. neutrophils, naïve T cells, macrophages, monocytes and hepatocytes), and a soluble form (soluble IL-6R, sIL-6R) which exists in the extracellular space (e.g. in the circulation) (Rose-John et al., supra).

IL-6 signalling through IL-6R can occur via two distinct pathways: classical and trans signalling. Both pathways are initiated by the interaction of circulating IL-6 cytokine with IL-6R and are dependent on the subsequent interaction between the IL-6/IL-6R complex and the ubiquitously expressed (e.g. expressed on essentially all cells), trans-membrane protein gp130 (Rose-John et al., supra). A soluble form of gp130 (sgp130) also exists in the extracellular space. Upon IL-6 binding to membrane-bound IL-6R or soluble IL-6R, dimerization of membrane-bound gp130 activates signal transduction through JAK tyrosine kinase family members, leading to phosphorylation of the transcription factor STAT3 on Y705 and its activation (Johnson et al., 2018, Nat. Rev. Clin. Oncol., 15(4):234-248). Activated STAT3, along with other activated transcription factors, induce expression of downstream target genes involved in differentiation, survival, apoptosis and proliferation, and can activate either pro-inflammatory or anti-inflammatory pathways depending on the signalling pathway used (i.e. classical or trans) (Rose-John et al., Johnson et al., supra).

For IL-6 classical signalling, signalling through membrane-bound IL-6R is restricted to cells expressing IL-6R on the cell surface. IL-6 classical signalling is recognised as being anti-inflammatory and playing an important role in protective, homeostatic and regenerative functions including proliferation, inhibition of apoptosis and defence against bacterial infection (Rose-John et al., supra).

For IL-6 trans signalling, IL-6 binds to soluble IL-6R in the extracellular space and subsequent binding to gp130 on the cell membrane stimulates IL-6 trans signalling. As gp130 is ubiquitously expressed, IL-6 trans signalling can occur in all (or essentially all) cells of the body. Such IL-6 trans signalling can also thus occur in cells that are not expressing the membrane-bound IL-6R. In other words, the expression of IL-6R on the cell surface is not required for trans signalling. IL-6 trans signalling is recognised as being pro-inflammatory and playing a significant role in pathological inflammation and cancer (Rose-John et al., supra).

A natural buffer system is believed to regulate extracellular IL-6 levels, where molecules which have the ability to interact with IL-6/soluble IL-6R trans-signalling complexes to form inactive complexes, for example soluble gp130, reduce free extracellular IL-6 levels and prevent promiscuous IL-6 signalling (Baran et al., J Biol Chem, 2018, 293(18), 6762-6775).

The important role of IL-6 trans signalling in disease has led to the development of antibodies and other types of biologics which inhibit IL-6 signalling, e.g. Sylvant (siltuximab), Actemra (tocilizumab) and Kevzara (sarilumab) are monoclonal antibodies targeting human IL-6 or IL-6R (Rose-John et al., Johnson et al., supra). These therapeutics block both IL-6 classical and trans signalling. Such antibodies can thus lead to side-effects for patients due to the inhibition of IL-6 classical signalling in addition to other functionally related cytokine signalling pathways (e.g. susceptibility to bacterial infection, neutropenia, malignancies, changes of blood counts, gastrointestinal perforations, hepatic manifestations and cardiovascular risk).

Antibodies selectively targeting murine IL-6 trans signalling have previously been described, however approaches to develop antibodies raised to the corresponding epitope on human IL-6R failed to selectively inhibit human IL-6 trans signalling, highlighting the complexities of developing antibodies that selectively inhibit IL-6 trans signalling in humans (Lacroix et al., J Biol Chem, 2015, 290(45), 26943-26953).

An alternative potential approach to inhibiting human IL-6 trans signalling targets the human soluble IL-6R/IL-6 complex. However, this approach can result in the unwanted inhibition of IL-6 classical signalling due to the trapping of IL-6 in IL-6 receptor complexes, and thus does not lead to selective inhibition of IL-6 trans signalling. Additionally, this approach results in the off-target inhibition of other signalling pathways which require gp130 for signal transduction, for example the inhibition of IL-11 signalling by trapping of IL-11 in IL-11 receptor complexes, and thus leads to unwanted and potentially detrimental off-target effects.

Other biologics targeting the human soluble IL-6R/IL-6 complex have also been described. For example VHH6, a single domain antibody which binds to the soluble IL-6R/IL-6 complex, was found to trap IL-6 in the soluble IL-6R/IL-6 complex resulting in an increase in IL-6 trans signalling, which would be expected to lead to local or systemic pathological inflammation (Baran et al., supra). Such trapping of IL-6 may also result in in the unwanted inhibition of IL-6 classical signalling as discussed above.

There is therefore a need for alternative and preferably improved therapeutics which have improved function in humans and, ideally, an improved side effect profile. Specifically, therapeutics are required that selectively inhibit human IL-6 trans signalling (i.e. do not inhibit, or do not significantly inhibit, IL-6 classical signalling) and preferably do not trap IL-6 in the soluble IL-6 receptor complex (or other inactive complexes) and/or do not inhibit other gp130-dependent signalling pathways, e.g. IL-11 signalling. Surprisingly, the present inventors have identified such antibodies which are able to bind to the human IL-6 receptor and selectively inhibit or target IL-6 trans signalling as opposed to IL-6 classical signalling in humans, e.g. are able to selectively inhibit or target IL-6 trans signalling without affecting IL-6 classical signalling in humans.

The present invention thus provides one such alternative and improved therapeutic option in the form of antibodies and binding proteins (antigen binding proteins) which bind to human IL-6R and inhibit IL-6 trans signalling while maintaining (e.g. not affecting) IL-6 classical signalling. The antibodies generated by the inventors thus have advantageous properties which make them ideal agents for the above-mentioned therapeutic uses and those described elsewhere herein.

As will be described elsewhere herein, antibodies of the invention have been shown to be capable of binding to human IL-6R and inhibiting IL-6 trans signalling through the human soluble IL-6R while maintaining (e.g. not significantly affecting or not significantly reducing) IL-6 classical signalling through the human membrane-bound IL-6R.

Thus, at its broadest, the present invention provides an antigen binding protein, for example an antibody, which binds to (is capable of binding to) human IL-6R and inhibits IL-6 trans signalling through the human soluble IL-6R while maintaining (e.g. not significantly affecting or not significantly reducing) IL-6 classical signalling (also sometimes referred to as “classic” signalling) through the human membrane-bound IL-6R.

Viewed alternatively, the present invention provides an antigen binding protein, for example an antibody, comprising at least one, preferably two, antigen binding domains that binds to human IL-6R, wherein said antigen binding protein or antibody inhibits IL-6 trans signalling through the human soluble IL-6R while maintaining IL-6 classical signalling through the human membrane-bound IL-6R.

The present invention further provides an antigen binding protein, for example an antibody, comprising at least one, preferably two, antigen binding domains that binds to human IL-6R, wherein said antigen binding protein or antibody inhibits IL-6 trans signalling through the human soluble IL-6R while maintaining IL-6 classical signalling through the human membrane-bound IL-6R, and wherein said antigen binding protein or antibody can bind to human soluble IL-6R and human membrane-bound IL-6R.

Such antibodies of the invention (or for example other binding proteins of the invention comprising a human IL-6R antigen binding domain as described herein) can conveniently and advantageously be used for the treatment of diseases associated with IL-6 signalling (preferably IL-6 trans signalling), in particular for the treatment of inflammatory diseases, CNS conditions, and cancer. By inhibiting IL-6 trans signalling while maintaining IL-6 classical signalling (e.g. by selectively or specifically targeting IL-6 trans signalling), treatment of said diseases can be achieved while preventing (or minimising or limiting) side effects associated with the inhibition of IL-6 classical signalling.

To the inventors' knowledge no other antibodies or binding proteins binding to human IL-6R have been disclosed to have the advantageous property of inhibiting IL-6 trans signalling while maintaining (e.g. not affecting or not significantly affecting or not significantly reducing) IL-6 classical signalling in humans.

In one embodiment, the present invention provides an antigen binding protein, for example an antibody, for example an isolated antibody (or antigen binding protein), comprising at least one, preferably two, antigen binding domains that binds to human IL-6R, wherein said antigen binding protein or antibody inhibits IL-6 trans signalling through the human soluble IL-6 receptor while maintaining (e.g. not significantly affecting or not significantly reducing) IL-6 classical signalling through the human membrane-bound IL-6 receptor, said antigen binding domain comprising a heavy chain variable region that comprises three complementarity determining regions (CDRs) and a light chain variable region that comprises three CDRs,
a) wherein said heavy chain variable region comprises:
(i) a variable heavy (VH) CDR1 that comprises the amino acid sequence of GFTFSSYA or a sequence substantially homologous thereto,
(ii) a VH CDR2 that comprises the amino acid sequence of INSNGGST or a sequence substantially homologous thereto, and
(iii) a VH CDR3 that comprises the amino acid sequence of AREGYYTMDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto; or
b) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of ARDGNYVSDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPWT or a sequence substantially homologous thereto; or
c) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of TRDGNFVSDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto; or
d) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of MNSKGGST or a sequence substantially homologous thereto, and
(iii) a VH CDR3 that comprises the amino acid sequence of ARDGYYTMDY or a sequence substantially homologous thereto; and/or (preferably “and”)
wherein said light chain variable region comprises:
(iv) a variable light (VL) CDR1 that comprises the amino acid sequence of KSVDSFGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto; or
e) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of ARDGNFVSDY or a sequence substantially homologous thereto; and/or (preferably “and”)
wherein said light chain variable region comprises:
(iv) a variable light (VL) CDR1 that comprises the amino acid sequence of ESVDSYGNRF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto.

In one embodiment, the present invention provides an antigen binding protein, for example an antibody, for example an isolated antibody (or antigen binding protein), comprising at least one, preferably two, antigen binding domains that binds to human IL-6R, wherein said antigen binding protein or antibody inhibits IL-6 trans signalling through the human soluble IL-6 receptor while maintaining (e.g. not significantly affecting or not significantly reducing) IL-6 classical signalling through the human membrane-bound IL-6 receptor, said antigen binding domain comprising a heavy chain variable region that comprises three complementarity determining regions (CDRs) and a light chain variable region that comprises three CDRs,
a) wherein said heavy chain variable region comprises:
(i) a variable heavy (VH) CDR1 that comprises the amino acid sequence of GFTFSSYA or a sequence substantially homologous thereto,
(ii) a VH CDR2 that comprises the amino acid sequence of INSNGGST or a sequence substantially homologous thereto, and
(iii) a VH CDR3 that comprises the amino acid sequence of AREGYYTMDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto;
wherein said substantially homologous sequence is a sequence containing 1, 2, or 3 amino acid substitutions compared to the given CDR sequences; or
b) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of ARDGNYVSDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPWT or a sequence substantially homologous thereto;
wherein said substantially homologous sequence is a sequence containing 1, 2, or 3 amino acid substitutions compared to the given CDR sequences; or
c) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of TRDGNFVSDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto;
wherein said substantially homologous sequence is a sequence containing 1, 2, or 3 amino acid substitutions compared to the given CDR sequences; or
d) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of MNSKGGST or a sequence substantially homologous thereto, and
(iii) a VH CDR3 that comprises the amino acid sequence of ARDGYYTMDY or a sequence substantially homologous thereto; and/or (preferably “and”)
wherein said light chain variable region comprises:
(iv) a variable light (VL) CDR1 that comprises the amino acid sequence of KSVDSFGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto;
wherein said substantially homologous sequence is a sequence containing 1, 2, or 3 amino acid substitutions compared to the given CDR sequences; or
e) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of ARDGNFVSDY or a sequence substantially homologous thereto; and/or (preferably “and”)
wherein said light chain variable region comprises:
(iv) a variable light (VL) CDR1 that comprises the amino acid sequence of ESVDSYGNRF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto;
wherein said substantially homologous sequence is a sequence containing 1, 2, or 3 amino acid substitutions compared to the given CDR sequences.

In one embodiment, the present invention provides an antigen binding protein, for example an antibody, for example an isolated antibody (or antigen binding protein), comprising at least one, preferably two, antigen binding domains that binds to human IL-6R, wherein said antigen binding protein or antibody inhibits IL-6 trans signalling through the human soluble IL-6 receptor while maintaining (e.g. not significantly affecting or not significantly reducing) IL-6 classical signalling through the human membrane-bound IL-6 receptor, said antigen binding domain comprising a heavy chain variable region that comprises three complementarity determining regions (CDRs) and a light chain variable region that comprises three CDRs,
a) wherein said heavy chain variable region comprises:
(i) a variable heavy (VH) CDR1 that comprises the amino acid sequence of GFTFSSYA or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of INSNGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of AREGYYTMDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of ESVDSYGNSF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPYT or a sequence substantially homologous thereto, wherein said substantially homologous sequence is a sequence containing 1, 2, or 3 amino acid substitutions compared to the given CDR sequence; or
b) wherein said heavy chain variable region comprises:
(i) a variable heavy (VH) CDR1 that comprises the amino acid sequence of GFTFSNYG or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of INSNGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of ARDGNYVSDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of ESVDSYGNSF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and (vi) a VL CDR3 that comprises the amino acid sequence of QQNNEDPWT or a sequence substantially homologous thereto, wherein said substantially homologous sequence is a sequence containing 1, 2, or 3 amino acid substitutions compared to the given CDR sequence; or
c) wherein said heavy chain variable region comprises:
(i) a variable heavy (VH) CDR1 that comprises the amino acid sequence of GFTFSNYG or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of INSNGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of TRDGNFVSDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of ESVDSYGNSF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPYT or a sequence substantially homologous thereto, wherein said substantially homologous sequence is a sequence containing 1, 2, or 3 amino acid substitutions compared to the given CDR sequence; or
d) wherein said heavy chain variable region comprises:
(i) a variable heavy (VH) CDR1 that comprises the amino acid sequence of GFTFSNYG or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of MNSKGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of ARDGYYTMDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of KSVDSFGNSF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPYT or a sequence substantially homologous thereto, wherein said substantially homologous sequence is a sequence containing 1, 2, or 3 amino acid substitutions compared to the given CDR sequence; or
e) wherein said heavy chain variable region comprises:
(i) a variable heavy (VH) CDR1 that comprises the amino acid sequence of GFTFSNYG or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of INSNGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of ARDGNFVSDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of ESVDSYGNRF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPYT or a sequence substantially homologous thereto, wherein said substantially homologous sequence is a sequence containing 1, 2, or 3 amino acid substitutions compared to the given CDR sequence.

In one embodiment, the present invention provides an antigen binding protein, for example an antibody, for example an isolated antibody (or antigen binding protein), comprising at least one, preferably two, antigen binding domains that binds to human IL-6R, said antigen binding domain comprising a heavy chain variable region that comprises three complementarity determining regions (CDRs) and a light chain variable region that comprises three CDRs,
a) wherein said heavy chain variable region comprises:
(i) a variable heavy (VH) CDR1 that comprises the amino acid sequence of GFTFSSYA or a sequence substantially homologous thereto,
(ii) a VH CDR2 that comprises the amino acid sequence of INSNGGST or a sequence substantially homologous thereto, and
(iii) a VH CDR3 that comprises the amino acid sequence of AREGYYTMDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto;
wherein said substantially homologous sequence is for example a sequence containing 1, 2, or 3 amino acid substitutions compared to the given CDR sequences; or
b) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of ARDGNYVSDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPWT or a sequence substantially homologous thereto;
wherein said substantially homologous sequence is for example a sequence containing 1, 2, or 3 amino acid substitutions compared to the given CDR sequences; or
c) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of TRDGNFVSDY or a sequence substantially homologous thereto; and/or (preferably “and”)
wherein said light chain variable region comprises:
(iv) a variable light (VL) CDR1 that comprises the amino acid sequence of ESVDSYGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto;
wherein said substantially homologous sequence is for example a sequence containing 1, 2, or 3 amino acid substitutions compared to the given CDR sequences; or
d) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of MNSKGGST or a sequence substantially homologous thereto, and
(iii) a VH CDR3 that comprises the amino acid sequence of ARDGYYTMDY or a sequence substantially homologous thereto; and/or (preferably “and”)
wherein said light chain variable region comprises:
(iv) a variable light (VL) CDR1 that comprises the amino acid sequence of KSVDSFGNSF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto;
wherein said substantially homologous sequence is for example a sequence containing 1, 2, or 3 amino acid substitutions compared to the given CDR sequences; or
e) wherein said heavy chain variable region comprises:
(i) a variable heavy (VH) CDR1 that comprises the amino acid sequence of GFTFSNYG or a sequence substantially homologous thereto,
(ii) a VH CDR2 that comprises the amino acid sequence of INSNGGST or a sequence substantially homologous thereto, and
(iii) a VH CDR3 that comprises the amino acid sequence of ARDGNFVSDY or a sequence substantially homologous thereto; and/or (preferably “and”)
wherein said light chain variable region comprises:
(iv) a variable light (VL) CDR1 that comprises the amino acid sequence of ESVDSYGNRF or a sequence substantially homologous thereto,
(v) a VL CDR2 that comprises the amino acid sequence of LAS or a sequence substantially homologous thereto, and
(vi) a VL CDR3 that comprises the amino acid sequence of QQNNEDPYT or a sequence substantially homologous thereto;
wherein said substantially homologous sequence is for example a sequence containing 1, 2, or 3 amino acid substitutions compared to the given CDR sequences.

In one embodiment, the present invention provides an antigen binding protein, for example an antibody, for example an isolated antibody (or antigen binding protein), comprising at least one, preferably two, antigen binding domains that binds to human IL-6R, said antigen binding domain comprising a heavy chain variable region that comprises three complementarity determining regions (CDRs) and a light chain variable region that comprises three CDRs,
a) wherein said heavy chain variable region comprises:
(i) a variable heavy (VH) CDR1 that comprises the amino acid sequence of GFTFSSYA or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of INSNGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of AREGYYTMDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of ESVDSYGNSF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPYT or a sequence substantially homologous thereto, wherein said substantially homologous sequence is a sequence containing 1, 2, or 3 amino acid substitutions compared to the given CDR sequence; or
b) wherein said heavy chain variable region comprises:
(i) a variable heavy (VH) CDR1 that comprises the amino acid sequence of GFTFSNYG or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of INSNGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of ARDGNYVSDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of ESVDSYGNSF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPWT or a sequence substantially homologous thereto, wherein said substantially homologous sequence is a sequence containing 1, 2, or 3 amino acid substitutions compared to the given CDR sequence; or
c) wherein said heavy chain variable region comprises:
(i) a variable heavy (VH) CDR1 that comprises the amino acid sequence of GFTFSNYG or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of INSNGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of TRDGNFVSDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of ESVDSYGNSF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPYT or a sequence substantially homologous thereto, wherein said substantially homologous sequence is a sequence containing 1, 2, or 3 amino acid substitutions compared to the given CDR sequence; or
d) wherein said heavy chain variable region comprises:
(i) a variable heavy (VH) CDR1 that comprises the amino acid sequence of GFTFSNYG or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of MNSKGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of ARDGYYTMDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of KSVDSFGNSF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPYT or a sequence substantially homologous thereto, wherein said substantially homologous sequence is a sequence containing 1, 2, or 3 amino acid substitutions compared to the given CDR sequence; or
e) wherein said heavy chain variable region comprises:
(i) a variable heavy (VH) CDR1 that comprises the amino acid sequence of GFTFSNYG or a sequence substantially homologous thereto, wherein said substantially homologous sequence is a sequence containing 1, 2, or 3 amino acid substitutions compared to the given CDR sequence,
(ii) a VH CDR2 that comprises the amino acid sequence of INSNGGST or a sequence substantially homologous thereto, wherein said substantially homologous sequence is a sequence containing 1, 2, or 3 amino acid substitutions compared to the given CDR sequence, and
(iii) a VH CDR3 that comprises the amino acid sequence of ARDGNFVSDY or a sequence substantially homologous thereto, wherein said substantially homologous sequence is a sequence containing 1, 2, 3 or 4 amino acid substitutions compared to the given CDR sequence; and/or (preferably “and”)
wherein said light chain variable region comprises:
(iv) a variable light (VL) CDR1 that comprises the amino acid sequence of ESVDSYGNRF or a sequence substantially homologous thereto, wherein said substantially homologous sequence is a sequence containing 1, 2, 3 or 4 amino acid substitutions compared to the given CDR sequence,
(v) a VL CDR2 that comprises the amino acid sequence of LAS or a sequence substantially homologous thereto, wherein said substantially homologous sequence is a sequence containing 1 amino acid substitution compared to the given CDR sequence, and
(vi) a VL CDR3 that comprises the amino acid sequence of QQNNEDPYT or a sequence substantially homologous thereto, wherein said substantially homologous sequence is a sequence containing 1, 2, or 3 amino acid substitutions compared to the given CDR sequence.

In a preferred embodiment, the present invention provides an antibody (or antigen binding protein), for example an isolated antibody (or antigen binding protein), comprising at least one, preferably two, antigen binding domains that binds to human IL-6R, wherein said antibody (or binding protein) inhibits IL-6 trans signalling through the human soluble IL-6 receptor while maintaining (e.g. not significantly affecting or not significantly reducing) IL-6 classical signalling through the human membrane-bound IL-6 receptor, said antigen binding domain comprising a heavy chain variable region that comprises three complementarity determining regions (CDRs) and a light chain variable region that comprises three CDRs,
a) wherein said heavy chain variable region comprises:
(i) a variable heavy (VH) CDR1 that comprises the amino acid sequence of GFTFSSYA,
(ii) a VH CDR2 that comprises the amino acid sequence of INSNGGST, and
(iii) a VH CDR3 that comprises the amino acid sequence of AREGYYTMDY; and/or (preferably “and”)
wherein said light chain variable region comprises:
(iv) a variable light (VL) CDR1 that comprises the amino acid sequence of ESVDSYGNSF,
(v) a VL CDR2 that comprises the amino acid sequence of LAS, and
(vi) a VL CDR3 that comprises the amino acid sequence of QQNNEDPYT; or
b) wherein said heavy chain variable region comprises:
(i) a variable heavy (VH) CDR1 that comprises the amino acid sequence of GFTFSNYG,
(ii) a VH CDR2 that comprises the amino acid sequence of INSNGGST, and
(iii) a VH CDR3 that comprises the amino acid sequence of ARDGNYVSDY; and/or (preferably “and”)
wherein said light chain variable region comprises:
(iv) a variable light (VL) CDR1 that comprises the amino acid sequence of ESVDSYGNSF,
(v) a VL CDR2 that comprises the amino acid sequence of LAS, and
(vi) a VL CDR3 that comprises the amino acid sequence of QQNNEDPWT; or
c) wherein said heavy chain variable region comprises:
(i) a variable heavy (VH) CDR1 that comprises the amino acid sequence of GFTFSNYG,
(ii) a VH CDR2 that comprises the amino acid sequence of INSNGGST, and
(iii) a VH CDR3 that comprises the amino acid sequence of TRDGNFVSDY; and/or (preferably “and”)
wherein said light chain variable region comprises:
(iv) a variable light (VL) CDR1 that comprises the amino acid sequence of ESVDSYGNSF,
(v) a VL CDR2 that comprises the amino acid sequence of LAS, and
(vi) a VL CDR3 that comprises the amino acid sequence of QQNNEDPYT; or
d) wherein said heavy chain variable region comprises:
(i) a variable heavy (VH) CDR1 that comprises the amino acid sequence of GFTFSNYG,
(ii) a VH CDR2 that comprises the amino acid sequence of MNSKGGST, and
(iii) a VH CDR3 that comprises the amino acid sequence of ARDGYYTMDY; and/or (preferably “and”)
wherein said light chain variable region comprises:
(iv) a variable light (VL) CDR1 that comprises the amino acid sequence of KSVDSFGNSF,
(v) a VL CDR2 that comprises the amino acid sequence of LAS, and
(vi) a VL CDR3 that comprises the amino acid sequence of QQNNEDPYT; or
e) wherein said heavy chain variable region comprises:
(i) a variable heavy (VH) CDR1 that comprises the amino acid sequence of GFTFSNYG,
(ii) a VH CDR2 that comprises the amino acid sequence of INSNGGST, and
(iii) a VH CDR3 that comprises the amino acid sequence of ARDGNFVSDY; and/or (preferably “and”)
wherein said light chain variable region comprises:
(iv) a variable light (VL) CDR1 that comprises the amino acid sequence of ESVDSYGNRF,
(v) a VL CDR2 that comprises the amino acid sequence of LAS, and
(vi) a VL CDR3 that comprises the amino acid sequence of QQNNEDPYT.

In another aspect, the present invention provides an antibody (or antigen binding protein), for example an isolated antibody (or antigen binding protein), comprising at least one, preferably two, antigen binding domains that binds to human IL-6R, wherein said antibody (or binding protein) inhibits IL-6 trans signalling through the human soluble IL-6 receptor while maintaining (e.g. not significantly affecting or not significantly reducing) IL-6 classical signalling through the human membrane-bound IL-6 receptor, said antigen binding domain comprising a heavy chain variable region that comprises three complementarity determining regions (CDRs) and a light chain variable region that comprises three CDRs,
wherein said heavy chain region comprises:
   a) a variable heavy (VH) CDR1 that comprises the amino acid sequence of GFTFSX₆YX₈, wherein X₈ and X₈ is any amino acid, wherein X₆ is preferably N or S, and/or X₈ is preferably G or A;
   b) a VH CDR2 that comprises the amino acid sequence of X₁NSX₄GGST, wherein X₁ and X₄ is any amino acid, wherein X₁ is preferably I or M, and/or X₄ is preferably N or K; and
   c) a VH CDR3 that comprises the amino acid sequence X₁RX₃GX₅X₆X₇X₈DY, wherein X₁, X₃, X₅, X₆, X₇, and X₃ is any amino acid, wherein X₁ is preferably A or T, X₃ is preferably D or E, X₅ is preferably N or Y, X₆ is preferably Y or F, X₇ is preferably V or T, and/or X₃ is preferably S or M,
      preferably wherein the VH CDR3 amino acid sequence comprises ARX₃GYYTMDY, wherein X₃ is any amino acid, preferably E or D, or wherein the VH CDR3 amino acid sequence comprises X₁RDGNX₆VSDY, wherein X₁ and X₆ is any amino acid, wherein X₁ is preferably A or T, and/or X₆ is preferably F or Y; and/or (preferably “and”)
wherein said light chain variable region comprises:
   d) a variable light (VL) CDR1 that comprises the amino acid sequence of X₁SVDSX₆GNX₉F, wherein X₁, X₆, and X₉ is any amino acid, wherein X₁ is preferably E or K, X₈ is preferably Y or F, and/or X₉ is preferably S or R;
   e) a VL CDR2 that comprises the amino acid sequence of LAS, or a sequence substantially homologous thereto, wherein said substantially homologous sequence is a sequence containing 1, 2 or 3 amino acid substitutions, preferably 1 amino acid substitution; and
   f) a VL CDR3 that comprises the amino acid sequence of QQNNEDPX₈T, wherein X₃ is any amino acid, preferably Y or W.

In some embodiments, the VH CDR1 has or comprises an amino acid sequence of GFTFSX₆YX₆. In these embodiments, X₆ and X₈ can be any amino acid. Preferably one or both of these X residues are selected from the following group: X₆ is N or S; X₈ is G or A. Thus, a preferred VH CDR1 has or comprises the amino acid sequence of GFTFS[N/S]Y[G/A]. For example, preferred VH CDR1 sequences of this embodiment have or comprise the sequence GFTFSNYG or GFTFSSYA.

In some embodiments, the VH CDR2 has or comprises an amino acid sequence of X₁NSX₄GGST. In these embodiments, X₁ and X₄ can be any amino acid. Preferably one or both of these X residues are selected from the following group: X₁ is I or M; X₄ is N or K.

Thus, a preferred VH CDR2 has or comprises the amino acid sequence of [I/M]NS[N/K]GGST. For example, preferred VH CDR2 sequences of this embodiment have or comprise the sequence INSNGGST or MNSKGGST.

In some embodiments, the VH CDR3 has or comprises an amino acid sequence of X₁RX₃GX₅X₆X₇X₃DY. In these embodiments, X₁, X₃, X₅, X₆, X₇ and X₃ can be any amino acid. Preferably one or more, e.g. one, two, three, four, five or six, most preferably all, of these X residues are selected from the following group: X₁ is A or T; X₃ is D or E; X₅ is N or Y; X₆ is Y or F; X₇ is V or T; X₈ is S or M. Thus, a preferred VH CDR3 has or comprises the amino acid sequence of [A/T]R[D/E]G[N/Y][Y/F][V/T][S/M]DY. For example, preferred VH CDR3 sequences of this embodiment have or comprise the sequence AREGYYTMDY, ARDGNYVSDY, TRDGNFVSDY, ARDGYYTMDY or ARDGNFVSDY.

In a preferred embodiment, the VH CDR3 has or comprises an amino acid sequence of ARX₃GYYTMDY. In this embodiment, X₃ can be any amino acid. Preferably the X residue is selected from the following group: X₃ is E or D. Thus, a preferred VH CDR3 has or comprises the amino acid sequence of AR[E/D]GYYTMDY. For example, preferred VH CDR3 sequences of this embodiment have or comprise the sequence AREGYYTMDY or ARDGYYTMDY.

In another preferred embodiment, the VH CDR3 has or comprises an amino acid sequence of X₁RDGNX₆VSDY. In this embodiment, X₁ and X₆ can be any amino acid. Preferably one or both of these X residues are selected from the following group: X₁ is A or T; X₆ is F or Y. Thus, an alternative preferred VH CDR3 has or comprises the amino acid sequence of [A/T]RDGN[F/Y]VSDY. For example, preferred VH CDR3 sequences of this embodiment have or comprise the sequence ARDGNYVSDY, TRDGNFVSDY or ARDGNFVSDY.

In some embodiments, the VL CDR1 has or comprises an amino acid sequence of X₁SVDSX₆GNX₉F. In these embodiments, X₁, X₆ and X₉ can be any amino acid. Preferably one or more, e.g. one, two, or three, most preferably all, of these X residues are selected from the following group: X₁ is E or K; X₆ is Y or F; X₉ is S or R. Thus, a preferred VL CDR1 has or comprises the amino acid sequence of [E/K]SVDS[Y/F]GN[S/R]F. For example, preferred VL CDR1 sequences of this embodiment have or comprise the sequence ESVDSYGNSF, KSVDSFGNSF or ESVDSYGNRF.

In some embodiments, the VL CDR2 has or comprises an amino acid sequence of LAS or a sequence substantially homologous thereto. A preferred substantially homologous sequence is a sequence containing 1, 2, or 3 amino acid substitutions, more preferably 1 or 2 amino acid substitutions, most preferably 1 amino acid substitution.

In some embodiments, the VL CDR3 has or comprises an amino acid sequence of QQNNEDPX₈T. In these embodiments, X₈ can be any amino acid. Preferably the X residue is selected from the following group: X₈ is Y or W. Thus, a preferred VL CDR3 has or comprises the amino acid sequence of QQNNEDP[Y/W]T. For example, preferred VL CDR3 sequences of this embodiment have or comprise the sequence QQNNEDPYT or QQNNEDPWT.

The present invention further provides an antigen binding protein, for example an antibody, as described in any of the above embodiments or aspects, wherein said antigen binding protein or antibody can bind to human soluble IL-6R and human membrane-bound IL-6R.

In some embodiments, antibodies (or antigen binding proteins) of the invention comprise one or more of the above CDRs, e.g. one, two, three, four, five or all six of the above CDR sequences. Preferred antibodies (antigen binding proteins) of the invention comprise all three of the VH CDRs and/or (preferably “and”) all three of the VL CDRs. Thus, particularly preferred antibodies (or antigen binding proteins or antigen binding domains) of the invention comprise six CDRs.

In some embodiments, the present invention provides an antibody (or antigen binding protein), for example an isolated antibody (or antigen binding protein), comprising at least one, preferably two, antigen binding domains that binds to human IL-6R, wherein said antibody (or antigen binding protein) inhibits IL-6 trans signalling through the soluble human IL-6 receptor while maintaining (e.g. not significantly affecting or not significantly reducing) IL-6 classical signalling through the human membrane-bound IL-6 receptor, said antigen binding domain comprising a heavy chain variable region that comprises three complementarity determining regions (CDRs) and a light chain variable region that comprises three CDRs,
(a) wherein said heavy chain variable region comprises the amino acid sequence of SEQ ID NO:3, or a sequence substantially homologous thereto (e.g. a sequence having at least 80% sequence identity thereto, for example at least 85%, 90%, 95% or 98% sequence identity thereto), and/or (preferably “and”)
   wherein said light chain variable region comprises the amino acid sequence of SEQ ID NO:4, or a sequence substantially homologous thereto (e.g. a sequence having at least 80% sequence identity thereto, for example at least 85%, 90%, 95% or 98% sequence identity thereto); or
(b) wherein said heavy chain variable region comprises the amino acid sequence of SEQ ID NO:21, or a sequence substantially homologous thereto (e.g. a sequence having at least 80% sequence identity thereto, for example at least 85%, 90%, 95% or 98% sequence identity thereto), and/or (preferably “and”)
   wherein said light chain variable region comprises the amino acid sequence of SEQ ID NO:22, or a sequence substantially homologous thereto (e.g. a sequence having at least 80% sequence identity thereto, for example at least 85%, 90%, 95% or 98% sequence identity thereto); or
(c) wherein said heavy chain variable region comprises the amino acid sequence of SEQ ID NO:39, or a sequence substantially homologous thereto (e.g. a sequence having at least 80% sequence identity thereto, for example at least 85%, 90%, 95% or 98% sequence identity thereto), and/or (preferably “and”)
   wherein said light chain variable region comprises the amino acid sequence of SEQ ID NO:40, or a sequence substantially homologous thereto (e.g. a sequence having at least 80% sequence identity thereto, for example at least 85%, 90%, 95% or 98% sequence identity thereto); or
(d) wherein said heavy chain variable region comprises the amino acid sequence of SEQ ID NO:57, or a sequence substantially homologous thereto (e.g. a sequence having at least 80% sequence identity thereto, for example at least 85%, 90%, 95% or 98% sequence identity thereto), and/or (preferably “and”)
   wherein said light chain variable region comprises the amino acid sequence of SEQ ID NO:58, or a sequence substantially homologous thereto (e.g. a sequence having at least 80% sequence identity thereto, for example at least 85%, 90%, 95% or 98% sequence identity thereto); or
(e) wherein said heavy chain variable region comprises the amino acid sequence of SEQ ID NO:75, or a sequence substantially homologous thereto (e.g. a sequence having at least 80% sequence identity thereto, for example at least 85%, 90%, 95% or 98% sequence identity thereto), and/or (preferably “and”)
   wherein said light chain variable region comprises the amino acid sequence of SEQ ID NO:76, or a sequence substantially homologous thereto (e.g. a sequence having at least 80% sequence identity thereto, for example at least 85%, 90%, 95% or 98% sequence identity thereto).

In another embodiment, the present invention provides an antibody (or antigen binding protein), for example an isolated antibody (or antigen binding protein), comprising at least one, preferably two, antigen binding domains that binds to human IL-6R, wherein said antibody (or antigen binding protein) inhibits IL-6 trans signalling through the human soluble IL-6 receptor while maintaining (e.g. not significantly affecting or not significantly reducing) IL-6 classical signalling through the human membrane-bound IL-6 receptor, said antigen binding domain comprising
(a) a heavy chain variable region that comprises the amino acid sequence of SEQ ID NO:3, or a sequence substantially homologous thereto (e.g. a sequence having at least 80% sequence identity thereto, for example at least 85%, 90%, 95% or 98% sequence identity thereto), further wherein said heavy chain variable region comprises three CDRs, preferably comprising the amino acid sequences of GFTFSSYA, INSNGGST and AREGYYTMDY, or sequences substantially homologous thereto, as defined elsewhere herein, and/or (preferably “and”) a light chain variable region that comprises the amino acid sequence of SEQ ID NO:4, or a sequence substantially homologous thereto (e.g. a sequence having at least 80% sequence identity thereto, for example at least 85%, 90%, 95% or 98% sequence identity thereto), further wherein said light chain variable region comprises three CDRs, preferably comprising the amino acid sequences of ESVDSYGNSF, LAS and QQNNEDPYT, or sequences substantially homologous thereto, as defined elsewhere herein; or
(b) a heavy chain variable region that comprises the amino acid sequence of SEQ ID NO:21, or a sequence substantially homologous thereto (e.g. a sequence having at least 80% sequence identity thereto, for example at least 85%, 90%, 95% or 98% sequence identity thereto), further wherein said heavy chain variable region comprises three CDRs, preferably comprising the amino acid sequences of GFTFSNYG, INSNGGST and ARDGNYVSDY, or sequences substantially homologous thereto, as defined elsewhere herein, and/or (preferably “and”) a light chain variable region that comprises the amino acid sequence of SEQ ID NO:22, or a sequence substantially homologous thereto (e.g. a sequence having at least 80% sequence identity thereto, for example at least 85%, 90%, 95% or 98% sequence identity thereto), further wherein said light chain variable region comprises three CDRs, preferably comprising the amino acid sequences of ESVDSYGNSF, LAS and QQNNEDPWT, or sequences substantially homologous thereto, as defined elsewhere herein; or
(c) a heavy chain variable region that comprises the amino acid sequence of SEQ ID NO:39, or a sequence substantially homologous thereto (e.g. a sequence having at least 80% sequence identity thereto, for example at least 85%, 90%, 95% or 98% sequence identity thereto), further wherein said heavy chain variable region comprises three CDRs, preferably comprising the amino acid sequences of GFTFSNYG, INSNGGST and TRDGNFVSDY, or sequences substantially homologous thereto, as defined elsewhere herein, and/or (preferably “and”) a light chain variable region that comprises the amino acid sequence of SEQ ID NO:40, or a sequence substantially homologous thereto (e.g. a sequence having at least 80% sequence identity thereto, for example at least 85%, 90%, 95% or 98% sequence identity thereto), further wherein said light chain variable region comprises three CDRs, preferably comprising the amino acid sequences of ESVDSYGNSF, LAS and QQNNEDPYT, or sequences substantially homologous thereto, as defined elsewhere herein; or
(d) a heavy chain variable region that comprises the amino acid sequence of SEQ ID NO:57, or a sequence substantially homologous thereto (e.g. a sequence having at least 80% sequence identity thereto, for example at least 85%, 90%, 95% or 98% sequence identity thereto), further wherein said heavy chain variable region comprises three CDRs, preferably comprising the amino acid sequences of GFTFSNYG, MNSKGGST and ARDGYYTMDY, or sequences substantially homologous thereto, as defined elsewhere herein, and/or (preferably “and”) a light chain variable region that comprises the amino acid sequence of SEQ ID NO:58, or a sequence substantially homologous thereto (e.g. a sequence having at least 80% sequence identity thereto, for example at least 85%, 90%, 95% or 98% sequence identity thereto), further wherein said light chain variable region comprises three CDRs, preferably comprising the amino acid sequences of KSVDSFGNSF, LAS and QQNNEDPYT, or sequences substantially homologous thereto, as defined elsewhere herein; or
(e) a heavy chain variable region that comprises the amino acid sequence of SEQ ID NO:75, or a sequence substantially homologous thereto (e.g. a sequence having at least 80% sequence identity thereto, for example at least 85%, 90%, 95% or 98% sequence identity thereto), further wherein said heavy chain variable region comprises three CDRs, preferably comprising the amino acid sequences of GFTFSNYG, INSNGGST and ARDGNFVSDY, or sequences substantially homologous thereto, as defined elsewhere herein, and/or (preferably “and”) a light chain variable region that comprises the amino acid sequence of SEQ ID NO:76, or a sequence substantially homologous thereto (e.g. a sequence having at least 80% sequence identity thereto, for example at least 85%, 90%, 95% or 98% sequence identity thereto), further wherein said light chain variable region comprises three CDRs, preferably comprising the amino acid sequences of ESVDSYGNRF, LAS and QQNNEDPYT, or sequences substantially homologous thereto, as defined elsewhere herein.

In a preferred embodiment, the present invention provides an antibody (or antigen binding protein), for example an isolated antibody (or antigen binding protein), comprising at least one, preferably two, antigen binding domains that binds to human IL-6R, wherein said antibody (or binding protein) inhibits IL-6 trans signalling through the human soluble IL-6 receptor while maintaining (e.g. not significantly affecting or not significantly reducing) IL-6 classical signalling through the human membrane-bound IL-6 receptor, said antigen binding domain comprising at least one heavy chain variable region that comprises three complementarity determining regions (CDRs) and at least one light chain variable region that comprises three CDRs,
(a) wherein said heavy chain variable region comprises the amino acid sequence of SEQ ID NO:3, and/or (preferably “and”)
   wherein said light chain variable region comprises the amino acid sequence of SEQ ID NO:4; or
(b) wherein said heavy chain variable region comprises the amino acid sequence of SEQ ID NO:21, and/or (preferably “and”)
   wherein said light chain variable region comprises the amino acid sequence of SEQ ID NO:22; or
(c) wherein said heavy chain variable region comprises the amino acid sequence of SEQ ID NO:39, and/or (preferably “and”)
   wherein said light chain variable region comprises the amino acid sequence of SEQ ID NO:40; or
(d) wherein said heavy chain variable region comprises the amino acid sequence of SEQ ID NO:57, and/or (preferably “and”)
   wherein said light chain variable region comprises the amino acid sequence of SEQ ID NO:58; or
(e) wherein said heavy chain variable region comprises the amino acid sequence of SEQ ID NO:75, and/or (preferably “and”)
   wherein said light chain variable region comprises the amino acid sequence of SEQ ID NO:76.

The present invention further provides an antibody (or antigen binding protein), as described in any of the above embodiments or aspects, wherein said antigen binding protein or antibody can bind to human soluble IL-6R and human membrane-bound IL-6R.

In another embodiment, the present invention provides an antibody (or antigen binding protein), for example an isolated antibody (or antigen binding protein), comprising at least one, preferably two, antigen binding domains that binds to human IL-6R, said antigen binding domain comprising at least one heavy chain variable region that comprises three complementarity determining regions (CDRs) and at least one light chain variable region that comprises three CDRs, wherein said heavy chain variable region and/or (preferably “and”) said light chain variable region, is as described elsewhere herein.

In alternative embodiments of the invention, sequences indicated as having sequence identity to a given sequence, e.g. a sequence with a given SEQ ID NO, can have at least 55%, 60%, 65%, 70% or 75% identity to the sequence, or at least 96%, 97% or 99% identity to the sequence. Other appropriate levels of sequence identity are provided elsewhere herein.

CDR sequences of certain antibodies of the invention are set forth herein in Tables A, B, C, D, E and F. In some other embodiments, CDR sequences of antibodies of the invention may be CDR sequences in the VH domains and VL domains of antibodies of the invention as identified using any suitable method (or tool), for example as identified using the IMGT numbering scheme (e.g. Lefranc, M.-P., The Immunologist, 7, 132-136 (1999); www.imqt.org)), e.g. as shown in Tables A, B, C, D and E, or Chothia (e.g. Chothia C, et al. (1989) Nature, 342:877-883, or Al-Lazikani et al., (1997) JMB 273, 927-948), or as identified according to the well-known methods of Kabat (e.g. Kabat, et al., “Sequences of Proteins of Immunological Interest”, 5th Ed. Public Health Service, National Institutes of Health, Bethesda, MD, 647-669, 1991), or by AbM numbering (e.g. Abhinandan and Martin, 2008, Mol. Immunol. 45:3832-3839).

Anti-IL-6R antibodies (and antigen binding proteins) based on the C07, C09, C12, C19 and C20 antibody sequences set forth in Tables A, B, C, D and E, respectively, are preferred. The CDR domains, FR domains, VH and VL domains are shown in Tables A, B, C, D and E herein. Antibodies (or antigen binding proteins) comprising these sets of CDR domains or VH and VL domains, or whole (or full length) antibody formats, such as IgG containing formats, e.g. IgG1, comprising such domains (or sequences substantially homologous thereto) are preferred embodiments of the invention.

Thus, a preferred antigen binding protein of the invention is or comprises the C07, C09, C12, C19 or C20 antibody (or the six CDRs thereof, or the VH and VL domains thereof) set forth in Tables A, B, C, D and E, respectively. Antigen binding proteins with sequences substantially homologous thereto as defined elsewhere herein are also preferred.

In some embodiments a preferred antigen binding protein of the invention is or comprises the C20 antibody (or the six CDRs thereof, or the VH and VL domains thereof), or sequences substantially homologous thereto.

In some embodiments a preferred antigen binding protein of the invention is or comprises the C20, C07, C12, or C19 antibody (or the six CDRs thereof, or the VH and VL domains thereof), or sequences substantially homologous thereto.

An antigen binding protein according to any aspect of the present invention and disclosure may be defined as a binding protein comprising an antigen-binding domain, e.g. comprising at least one antigen binding domain, obtained or derived from an antibody, or based on or corresponding to an antigen binding domain of an antibody. Thus, for example, light and heavy chain variable domains (i.e. light and heavy chain variable regions) as described herein are those obtained or derived from an antibody, or based on or corresponding to an antigen binding domain of an antibody. Put another way, an antigen binding domain as referred to herein generally comprises a heavy chain variable region that comprises three CDRs and a light chain variable region that comprises three CDRs.

As described above, the present invention provides antigen binding proteins, for example antibodies, or antigen binding proteins comprising antibodies or the antigen binding domain of an antibody, which bind to (or specifically recognise or specifically bind to) human IL-6R. Preferred antigen binding proteins of the invention are antibodies or an antigen binding fragment thereof (also referred to as antibody fragments, e.g. Fab fragments). The term “antibody” is used as shorthand to refer to “antibody or an antigen binding fragment of an antibody” unless otherwise clear from context.

However, embodiments as described herein which relate to antibodies, apply equally, mutatis mutandis, to other types of antigen binding proteins, or vice versa. Thus, other antigen binding proteins can comprise the antibodies of the invention or can comprise the antigen binding domains of the antibodies of the invention, e.g. the three VL CDR regions and the three VH CDR regions of the antibodies of the invention, or a VL and VH domain of the antibodies of the invention (a domain typically comprising the three VH or the three VL CDR regions (CDR1, CDR2 and CDR3) and the four VH or the four VL framework (“FR”) regions (FR1, FR2, FR3 and FR4) as appropriate).

Preferred antigen binding proteins are any polypeptide chains which can bind (e.g. specifically bind) to human IL-6R. Appropriate types of antigen binding protein which could be used in the invention are known in the art. For example, in some embodiments immunoglobulin-based polypeptides are used, which generally comprise CDR regions (and optionally FR regions or an immunoglobulin based scaffold), such that the CDR regions (and optionally FR regions) of the antibodies of the invention can be grafted onto an appropriate scaffold or framework, e.g. an immunoglobulin scaffold. Alternatively, the antigen binding domains or the antibodies of the invention can be incorporated into any appropriate antigen binding fragment or antibody containing format.

Other embodiments are Immunoglobulin (Ig) forms, e.g. IgG, IgA, IgD, IgE or IgM forms, or forms containing all or part of an immunoglobulin constant region, e.g. all or part of an IgG, IgA, IgD, IgE or IgM constant region, of the various antibodies defined herein, for example full length (or whole) Ig or IgG, IgM or IgA forms. IgG forms, preferably full length (or whole) IgG forms (e.g. IgG1, IgG2, IgG3 or IgG4 forms), of the antibodies of the invention as described herein are preferred, with IgG1 most preferred. Full length IgG antibodies typically comprise two substantially identical heavy chains (with appropriate variable and constant regions) and two substantially identical light chains (with appropriate variable and constant regions). In preferred embodiments the antibodies, e.g. the IgG antibodies, are monoclonal.

Said IgG (or other Ig) forms comprise heavy chain variable regions (VH) and light chain variable regions (VL) as described herein, and further comprise appropriate IgG (or other Ig) heavy and light chain constant regions, e.g. comprise an IgG (or other Ig) Fc region. The sequences of such constant regions are well known and described in the art and any of these may be used. Thus, these regions can be derived from any appropriate source or species, e.g. mouse or human. Preferably such IgG (or other Ig) sequences are human IgG (or other) sequences, most preferably human IgG1 sequences.

Other embodiments are antigen binding fragments (e.g. antibody fragments) of the antibodies of the invention described herein, e.g. fragments of C07, C09, C12, C19 and C20 antibodies (or antibodies based thereon, e.g. substantially homologous antibodies). Any type of antigen binding fragment is envisaged, providing it comprises one or more antigen binding domains of the invention. i.e. one or more antigen binding domains that bind to human IL-6R. Exemplary types of antigen-binding fragments are described elsewhere herein. In some embodiments Fab fragments are preferred. Fab fragments typically comprise the variable region of one heavy chain (together with an appropriate heavy chain constant region or CH1 region) and the variable region of one light chain (together with an appropriate light chain constant region) and comprise one antigen binding domain. F(ab′)2 fragments may also be used. Thus, in some embodiments the antibody is a Fab fragment or a F(ab′)2 fragment, preferably a Fab fragment, comprising the CDR sequences and/or heavy chain variable domain and/or light chain variable domain (or sequences substantially homologous thereto) as described in Tables A, B, C, D and E. In preferred embodiments the Fab fragments are monoclonal.

Thus, in some embodiments, the present invention provides a Fab fragment which binds to (or is capable of binding to) human IL-6R and inhibits IL-6 trans signalling through the human soluble IL-6R while maintaining IL-6 classical signalling through the human membrane-bound IL-6R. Preferably, the Fab fragment binds to (or is capable of binding to) human soluble IL-6R and human membrane-bound IL-6R. Other aspects of the invention apply mutatis mutandis to this aspect of the invention.

A particularly preferred Fab fragment comprises the 6 CDR sequences of the C20 antibody (SEQ ID NO's 77-82) and/or the heavy chain variable domain and/or light chain variable domain sequences (SEQ ID NO's 75 and 76), or sequences substantially homologous thereto. These sequences are also shown in Table E.

Other preferred Fab fragments comprise the 6 CDR sequences and/or the heavy chain variable domain and/or light chain variable domain sequences of the C07, C12, C19 or C09 antibodies as shown in Table A, C, D or B, or sequences substantially homologous thereto.

In some embodiments, antigen binding proteins or antibodies of the present invention are covalently or non-covalently conjugated to molecules which confer advantageous properties, such as reduced immunogenicity and/or increased circulation half-life. Said modifications are known to the skilled person, for example PEGylation.

In some embodiments, antigen binding proteins or antibodies of the present invention are PEGylated.

The term “PEGylation” is used herein to describe the process of both covalent and non-covalent attachment or amalgamation of polyethylene glycol (PEG) polymer chains to antibodies or binding proteins, which are then described as “PEGylated”. As will be known to the skilled person, the association of PEG to antibodies or binding proteins is known to form a stable hydration layer which can reduce immunogenicity and prolong circulation half-life by limiting clearance by the renal system, whilst antigen binding is maintained.

The CDRs of the antigen binding proteins or antibodies of the invention are preferably separated by appropriate framework regions such as those found in naturally occurring antibodies and/or effective engineered antibodies. Thus, the VH, VL and individual CDR sequences of the invention are preferably provided within or incorporated into an appropriate framework or scaffold to enable antigen (here human IL-6R) binding. Such framework sequences or regions may correspond to naturally occurring framework regions, FR1, FR2, FR3 and/or FR4, as appropriate, to form an appropriate scaffold, or may correspond to consensus framework regions, for example identified by comparing various naturally occurring framework regions. Alternatively, non-antibody scaffolds or frameworks, e.g. T cell receptor frameworks can be used.

Appropriate sequences that can be used for framework regions are well known and documented in the art and any of these may be used. Exemplary sequences for framework regions are one or more of the framework regions making up the VH and/or VL domains of the antibodies (or antigen binding proteins) of the invention, e.g. one or more of the framework regions of the C07, C09, C12, C19 or C20 antibodies as disclosed in Tables A or B or C or D or E, respectively, or framework regions substantially homologous thereto, and in particular framework regions that allow the maintenance of antigen specificity, for example framework regions that result in substantially the same or the same 3D structure of the antibody.

In certain embodiments, all four of the variable heavy chain and/or variable light chain framework regions (FR) (e.g. as disclosed in Tables A, B, C, D and E), as appropriate, or FR regions substantially homologous thereto, are found in the antibodies (or binding proteins) of the invention.

In some embodiments, the antibodies of the invention can be humanized antibodies. “Humanized” antibodies, which are based on substantially non-human variable region domains, are antibodies in which certain amino acids have been changed to better correspond with the amino acids typically present in human antibodies. Methods for generating humanized antibodies are well known in the art. For example, humanized antibodies can be accomplished by inserting the appropriate CDRs (e.g. murine/mouse CDRs such as those present in the antibodies of the present invention) into a human antibody “scaffold”. In some cases, one or more CDR residues may be changed to better correspond with the amino acids typically present in human antibodies. Thus, in some embodiments, CDRs of the invention, e.g. a set of 6 CDRs of the antibodies of the invention, e.g. from the exemplary C07, C09, C12, C19 or C20 antibodies of the present invention or sequences substantially homologous thereto, are present within (or combined with or inserted or grafted into) a human antibody framework, e.g. using appropriate FR regions as found in human antibodies.

The exemplary C07, C09, C12, C19 and C20 antibodies of the present invention comprise murine/mouse VH and VL domains. Thus, in certain embodiments, chimeric antibodies are preferred. Such antibodies typically comprise murine/mouse VH and VL domains such as those with SEQ ID NOs: 3, 4, 21, 22, 39, 40, 57, 58, 75 and 76 (and related, e.g. substantially homologous sequences as described herein) together with constant regions from another (non-mouse/murine) species, preferably human constant regions e.g. including human Fc regions, more preferably human Ig regions such as IgG, e.g. IgG1, constant regions. Thus, chimeric human antibodies are preferred.

The exemplary antibodies of the present invention (C07, C09, C12, C19 and C20), that bind to human IL-6R and inhibit IL-6 trans signalling while maintaining IL-6 classical signalling, were generated using a subtractive immunisation protocol followed by phage display. The subtractive immunisation technique is for example described in Sandrock, 1987, J. Immunol Methods, 100:73-82, and a particular protocol is provided in the Examples section herein involving tolerizing cycles of IP injections with cells which express human membrane-bound IL-6R (e.g. JV1 cells) followed by treatment with a cytotoxic agent (e.g. cyclophosphamide treatment), and IP immunizing injections with recombinant human soluble IL-6R alpha. Although other methods may be used, such subtractive immunisation protocols have been found to be particularly appropriate for generating antibodies with the described functional properties. Although phage display is a preferred technique for use in combination with such subtractive immunisation protocols, other techniques can be used for antibody screening following the subtractive immunisation.

Thus, antibodies (or antigen binding proteins) of the present invention bind to (or specifically recognise or specifically bind to) human IL-6R. Such antibodies (or antigen binding proteins) may also bind to IL-6R from other species, e.g. other mammalian species. However, as described elsewhere herein, in some embodiments the antibodies (or antigen binding proteins) of the present invention do not bind to (or do not significantly bind to) murine (or mouse) IL-6R.

The antibodies (or antigen binding proteins) can bind to any appropriate forms of human IL-6R, such as human soluble IL-6R and/or (preferably “and”) human membrane-bound IL-6R, specific examples of which are described elsewhere herein. Such binding is preferably dose-dependent binding, e.g. increasing concentrations of antibody (or binding protein) show increasing levels of binding to human IL-6R.

In preferred embodiments, antibodies (or antigen binding proteins) of the present invention bind to (or specifically recognise or specifically bind to) human soluble IL-6R. Soluble IL-6R (also sometimes referred to as the IL-6R alpha subunit) is generated by limited proteolysis of membrane-bound IL-6R by the protease ADAM17, or by alternative splicing, and soluble IL-6R in the extracellular space (upon IL-6 binding) can stimulate trans signalling on all cell types (as gp130 is ubiquitously expressed). IL-6 trans signalling through the soluble IL-6R is recognised as fundamental in pathological inflammation and cancer and is a target for therapy.

Thus, in certain embodiments the antibodies (or antigen binding proteins) of the invention can bind human soluble IL-6R. Such antibodies (or antigen binding proteins) may also bind to sIL-6R from other species, e.g. other mammalian species. However, as described elsewhere herein, in some embodiments the antibodies (or antigen binding proteins) of the present invention do not bind to (or do not significantly bind to) murine (or mouse) soluble IL-6R.

The antibodies (or antigen binding proteins) can bind to any appropriate forms of human soluble IL-6R. Preferred forms of human soluble IL-6R to which the antibodies (or antigen binding proteins) of the invention can bind include recombinant human soluble IL-6R, or a native or natural form of human soluble IL-6R, for example human soluble IL-6R when present in circulation. Such binding is preferably dose-dependent binding, e.g. increasing concentrations of antibody (or binding protein) show increasing levels of binding to human soluble IL-6R.

The sequences of human soluble IL-6R are well known and described in the art. An exemplary sequence is provided herein (SEQ ID NO:113). Recombinant human soluble IL-6R is also commercially available (e.g. PeproTech, catalogue number: 200-06RC).

In some embodiments, antibodies (or antigen binding proteins) of the present invention bind to (or specifically recognise or specifically bind to) human membrane-bound IL-6R. Membrane-bound IL-6R (which is typically composed of a complex of IL-6R/IL-6R alpha) and gp130/IL-6R beta) is expressed on the cell surface of a limited number of cells (e.g. neutrophils, naïve T cells, macrophages, monocytes and hepatocytes). IL-6 classical signalling through membrane-bound IL-6R is restricted to cells expressing IL-6R on the cell surface and is recognised as playing an important role in protective, homeostatic and regenerative functions including proliferation, inhibition of apoptosis and defence against bacterial infection.

Thus, in certain embodiments the antibodies (or antigen binding proteins) of the invention can bind human membrane-bound IL-6R. Such antibodies (or antigen binding proteins) may also bind to membrane-bound IL-6R from other species, e.g. other mammalian species. However, as described elsewhere herein, in some embodiments the antibodies (or antigen binding proteins) of the present invention do not bind to (or do not significantly bind to) murine (or mouse) membrane-bound IL-6R.

The antibodies (or antigen binding proteins) can bind to any appropriate forms of human membrane-bound IL-6R. Preferred forms of human membrane-bound IL-6R to which the antibodies (or antigen binding proteins) of the invention can bind include native or natural forms of human membrane-bound IL-6R, for example human IL-6R when present on the cell surface (e.g. human IL-6R endogenously expressed or naturally/natively expressed on the cell surface), or human membrane-bound IL-6R expressed (or over-expressed) on the cell surface, e.g. by recombinant or genetic engineering of cells that do not naturally express membrane-bound IL-6R. Such binding is preferably dose-dependent binding, e.g. increasing concentrations of antibody (or binding protein) show increasing levels of binding to human membrane-bound IL-6R.

The sequences of human membrane-bound IL-6R are well known and described in the art and can be obtained from various sequence databases, e.g. Uniprot entry P08887 provides sequences of human IL-6R subunit alpha (SEQ ID NO: 112) and Uniprot entry P40189 provides sequences of human IL-6R subunit beta (gp130) (SEQ ID NO:114).

Thus, the antibodies (or binding proteins) of the invention bind to human soluble IL-6R and/or (preferably “and”) human membrane-bound IL-6R and have the ability to inhibit IL-6 trans signalling through the human soluble IL-6 receptor while maintaining IL-6 classical signalling through the human membrane-bound IL-6 receptor.

Methods of assessing antibody (or antigen binding protein) binding to (or ability to bind to) human soluble or membrane-bound IL-6R may be any appropriate method and would be well-known to a person skilled in the art, for example flow cytometry to determine binding to membrane-bound IL-6R and an ELISA assay to determine binding to soluble IL-6R.

Thus, in some embodiments, binding of antibodies (or antigen binding proteins) to (or ability to bind to) human soluble IL-6R may be assessed using an ELISA assay. The skilled person will be familiar with ELISA assays and readily able to establish suitable conditions to assess the ability of an antibody (or antigen binding protein) to bind to soluble IL-6R in such an assay. For example, in a preferred assay, the ELISA plate is first coated with streptavidin presented with biotinylated recombinant IL-6 (to enable proper folding of soluble IL-6R) and subsequently incubated with recombinant human soluble IL-6R. The plates are then incubated with different concentrations of antibodies (or antigen binding proteins) in any appropriate format, e.g. full length (whole) antibodies (or antigen binding proteins), followed by subsequent detection and quantification of bound antibody. Typically, antibodies (or antigen binding proteins) of the present invention can bind to recombinant human soluble IL-6R in an ELISA assay, such as for example the ELISA assay described above. In a preferred embodiment, antibodies (or antigen binding proteins) of the present invention show dose-dependent binding to recombinant human soluble IL-6R in an ELISA assay, such as for example the ELISA assay described above. In preferred embodiments, antibodies (or antigen binding proteins) of the present invention can bind to (or significantly bind to, or are detected as binding to) recombinant human soluble IL-6R in an ELISA assay, such as for example the ELISA assay described above, when present at a concentration of at or at least 1 ng/ml, 10 ng/ml, 20 ng/ml, 50 ng/ml, 100 ng/ml or 500 ng/ml. Such values may be appropriate for example when the antibodies are in a full length (whole) antibody format, e.g. full length chimeric antibodies. Such binding is conveniently measured in comparison with an appropriate control, such as no antibody or an irrelevant control antibody (e.g. a non-IL-6R binding antibody).

In some embodiments, binding of antibodies (or antigen binding proteins) to (or ability to bind to) human membrane-bound IL-6R may be assessed using flow cytometry. The skilled person will be familiar with flow cytometry assays and readily able to establish suitable conditions to assess the ability of an antibody (or antigen binding protein) to bind to human IL-6R on the cell surface (membrane-bound IL-6R) in such an assay. For example, in a preferred assay, cells expressing membrane-bound IL-6R are cultured in the presence of or absence of recombinant IL-6 and incubated with antibodies (or antigen binding proteins) directly or indirectly conjugated to a fluorescent label, followed by analysis (or detection or quantification or fluorescence quantification) by flow cytometry. In a preferred embodiment, the cells used to assess binding of antibodies (or antigen binding proteins) to (or ability to bind to) human membrane-bound IL-6R are human THP1 cells (human monocytic cell line derived from a leukemia patient) or Ba/F3_gp130_IL-6R cells (a murine pre-B cell line genetically engineered to express human gp130 and human membrane-bound IL-6R), which both express human IL-6R on the cell surface. Typically, antibodies (or antigen binding proteins) of the present invention can bind to (or significantly bind to, or are detected as binding to) human membrane-bound IL-6R in a flow cytometry assay both in the presence of or absence of IL-6, such as for example the assay described above. Such binding is conveniently measured in comparison with an appropriate control, e.g. an increase in mean fluorescence in cells stained with antibodies of the invention and a secondary antibody when compared to cells stained with a secondary antibody only.

In some embodiments, the antibodies (or antigen binding proteins) of the invention do not bind (or do not significantly bind) to IL-6R (e.g. do not bind to, or do not significantly bind to, soluble and/or (preferably “and”) membrane-bound IL-6R) in non-human species, e.g. murine (or mouse), and/or (preferably “and”) do not affect or inhibit (or significantly affect or inhibit) IL-6 classical signalling or IL-6 trans signalling in non-human species, e.g. murine (or mouse). In other words, preferred antibodies (or antigen binding proteins) of the invention do not cross-react with IL-6R (e.g. do not cross-react with, or do not significantly cross-react with, soluble and/or (preferably “and”) membrane-bound IL-6R) in non-human species, e.g. murine IL-6R (or mouse IL-6R). The exemplified antibodies of the invention have been shown to not bind (or not significantly bind) to murine/mouse soluble IL-6R and murine/mouse membrane-bound IL-6R. In addition, the exemplified antibodies of the invention have been shown to not affect (or not significantly affect) IL-6 classical signalling or IL-6 trans signalling through the murine/mouse IL-6R.

The definitions for “maintains (or does not inhibit or does not significantly inhibit) IL-6 classical signalling” as used elsewhere herein apply mutatis mutandis to “does not affect or inhibit (or significantly affect or inhibit) IL-6 classical signalling or IL-6 trans signalling in non-human species, e.g. murine (or mouse)” as used herein.

The sequences of murine/mouse membrane-bound IL-6R are well known and described in the art and can be obtained from various sequence databases, e.g. Uniprot entry P22272 provides sequences of murine/mouse IL-6R subunit alpha (SEQ ID NO:115).

The sequences of murine/mouse soluble IL-6R are well known and described in the art. An exemplary sequence is provided herein (SEQ ID NO:116). Recombinant murine/mouse soluble IL-6R is also commercially available (e.g. Bio-techne, catalogue number: 1830-SR).

The antibodies (or antigen binding proteins) of the invention typically show the ability to bind to human IL-6R, e.g. soluble and/or (preferably “and”) membrane-bound human IL-6R, in the presence or the absence of IL-6, i.e. can bind to human IL-6R, e.g. soluble and/or (preferably “and”) membrane-bound human IL-6R, which is complexed to IL-6 or which is not complexed to IL-6. For example, the antibodies (or antigen binding proteins) of the invention typically show the ability to bind to human membrane IL-6R, in the presence or the absence of IL-6. In other words, the presence or absence of IL-6 does not affect the ability of the antibodies (or antigen binding proteins) to bind to human IL-6R, e.g. human membrane-bound IL-6R and/or human soluble IL-6R.

The antibodies (or antigen binding proteins) of the invention have the ability to bind to the human IL-6R (and were selected for this ability). Thus, the binding site (or epitope) of the antibodies (or antigen binding proteins) of the invention is located on the human IL-6R (e.g. soluble and/or membrane human IL-6R) or on the human IL-6R (e.g. soluble and/or membrane human IL-6R) alone. In other words, the binding site (or epitope) does not comprise any human IL-6 residues (or only comprises human IL-6R residues). Thus, the antibodies (or antigen binding proteins) of the invention have the ability to bind to the human IL-6R alone, e.g. show measurable and/or significant binding (e.g. binding at functionally, biologically or clinically relevant levels of affinity) to human IL-6R (e.g. soluble and/or membrane human IL-6R) alone.

Thus, it is believed that the dominant epitope (or main epitope or preferred epitope) bound by the antibodies (or antigen binding proteins) of the invention is located on the human IL-6R (e.g. soluble and/or membrane-bound human IL-6R). In other words, the dominant epitope (or main epitope or preferred epitope) does not comprise any human IL-6 residues (or only comprises human IL-6R residues).

More specifically, it is believed that the antibodies (or antigen binding proteins) of the invention bind to, or specifically bind to, an epitope (or dominant epitope, or main epitope, or preferred epitope) that is exclusively located (or wholly located or entirely located) on the human IL-6R (e.g. soluble and/or membrane-bound human IL-6R). In other words, the epitope does not comprise any human IL-6 residues (or only comprises human IL-6R residues).

Thus, in some embodiments, the antibodies (or antigen binding proteins) of the invention do not preferentially bind to human IL-6/IL-6R complex (e.g. soluble and/or membrane-bound human IL-6/IL-6R complex) as compared to human IL-6R (e.g. soluble and/or membrane-bound human IL-6R) alone. In other words, preferred antibodies (or antigen binding proteins) of the invention do not bind to human IL-6/IL-6R complex (e.g. soluble and/or membrane-bound human IL-6/IL-6R complex) to a greater extent or with a greater affinity, preferably a significantly greater extent or a significantly greater affinity, than the antibodies (or antigen binding proteins) bind to human IL-6R (e.g. soluble and/or membrane-bound human IL-6R) alone, e.g. when measured by an appropriate assay as described elsewhere herein (e.g. flow cytometry for membrane-bound IL-6R or membrane-bound IL-6/IL-6R complex, or an ELISA assay for soluble IL-6R or soluble IL-6/IL-6R complex). In other words, IL-6 (the presence of IL-6) does not influence or affect, preferably does not significantly influence or affect, binding of the preferred antibodies (or antigen binding proteins) of the invention to human IL-6R (soluble and/or membrane-bound human IL-6R).

As used herein the term “human IL-6R alone”, e.g. soluble and/or membrane-bound human IL-6R alone, refers to human IL-6R when not complexed with another molecule, e.g. when not complexed with IL-6/human IL-6. This term can also refer to an empty or unloaded human IL-6R.

In general therefore, the antibodies (or antigen binding proteins) of the invention do not bind (or do not significantly bind) to IL-6 or human IL-6 alone. As used herein the term “IL-6 alone” or “human IL-6 alone”, refers to IL-6 or human IL-6 when not complexed with another molecule, e.g. when not complexed with IL-6R/human IL-6R. This term can also refer to free IL-6.

As used herein the term “dominant epitope”, or “main epitope” or “preferred epitope” or “primary epitope”, etc., or equivalent terms, can refer to the epitope involved in the best or highest affinity binding of an antibody (or antigen binding protein) to human IL-6R, e.g. human soluble IL-6R and/or human membrane-bound IL-6R.

As the exemplified antibodies of the invention display similar functional properties (e.g. binding to soluble and membrane-bound human IL-6R, and inhibiting IL-6 trans signalling while maintaining IL-6 classical signalling, as defined elsewhere herein), and also have related sequences, e.g. related CDR sequences, it is believed that the exemplified antibodies of the invention may bind to the same epitope (e.g. a common epitope) on the IL-6R. Given that the functional properties of the exemplified antibodies have not been previously described, it is believed that the epitope(s) bound by the antibodies (or antigen binding proteins) of the invention is a unique and previously undisclosed epitope(s).

Without wishing to be bound by theory, it is believed that the antibodies (or antigen binding proteins) of the invention bind to, or specifically bind to, an epitope on IL-6R that is not involved in, or not significantly involved in, or not required for, IL-6/IL-6R complex (e.g. soluble and/or membrane IL-6/IL-6R complex) formation. In other words, it is believed that the antibodies (or antigen binding proteins) of the invention bind to, or specifically bind to, an epitope of IL-6R that is not involved in, or not significantly involved in, or not required for, IL-6 binding.

As such, it is believed that the antibodies (or antigen binding proteins) of the present invention do not trap, or significantly trap, IL-6 in complexes with soluble IL-6R or other inactive complexes, and thus the antibodies (or antigen binding proteins) do not affect (or do not significantly affect) the extracellular IL-6 buffering system. In other words, the antibodies (or antigen binding proteins) do not affect (or do not significantly affect) the equilibrium of extracellular IL-6, and/or do not reduce the level of or amount of (or do not significantly reduce the level of or amount of) free IL-6 in the extracellular space.

As used herein the term “trap” refers to IL-6 being complexed with other molecules (e.g. soluble IL-6R and soluble gp130), whereby the complexed IL-6 is no longer free to interact with other signalling molecules/transducers and induce other signalling pathways.

As used herein the term “IL-6 buffering system” and “equilibrium” refers to the ratio of, or relative concentration of, free IL-6 to IL-6 complexed with other molecules, which determines the amount of or level of free IL-6 in the extracellular space.

Thus, in some embodiments, the antibodies (or antigen binding proteins) of the present invention have the ability to bind to the human soluble IL-6R and/or (preferably “and”) the membrane-bound human IL-6R, and inhibit IL-6 trans signalling while maintaining IL-6 classical signalling, and/or (preferably “and”) do not affect (or do not significantly affect) the IL-6 buffering system (or do not reduce the level of or amount of (or do not significantly reduce the level of or amount of) free IL-6 in the extracellular space).

The unwanted off-target inhibition of IL-6 classical signalling due to IL-6 being trapped in complex with other molecules (e.g. soluble IL-6R and soluble gp130), becomes apparent under certain physiological conditions (e.g. when the extracellular concentration of soluble IL-6R exceeds the extracellular concentration of IL-6). The exemplified antibodies of the present invention have been shown to maintain IL-6 classical signalling (i.e. not inhibit or not significantly inhibit IL-6 classical signalling) under these experimental conditions (i.e. when the concentration of soluble IL-6R exceeds the concentration of IL-6). Thus, it is believed that the exemplified antibodies of the invention do not display the property of inhibition of IL-6 classical signalling due to trapping of IL-6, as defined elsewhere herein.

Thus, in some embodiments, the antibodies (or antigen binding proteins) of the invention inhibit IL-6 trans signalling while maintaining IL-6 classical signalling, under the conditions where (e.g. experimental conditions, e.g. of a cell-based IL-6 proliferation assay as described elsewhere herein) an excess concentration of soluble IL-6R as compared to IL-6 (i.e. a higher concentration of soluble IL-6R to IL-6 is used).

In some embodiments, the antibodies (or antigen binding proteins) of the invention have the ability to inhibit IL-6 trans signalling (e.g. inhibit IL-6 trans signalling through (or induced by or mediated by or associated with) the human soluble IL-6R). In some embodiments such inhibition is dose-dependent inhibition, e.g. increasing concentrations of antibody (or binding protein) show increasing levels of inhibition of IL-6 trans signalling.

As described elsewhere herein, IL-6 trans signalling through the soluble IL-6R is recognised as being pro-inflammatory and contributing to pathological inflammation and cancer. Thus, inhibition of IL-6 trans signalling by antibodies (or antigen binding proteins) of the present invention may advantageously inhibit (or reduce or limit) pro-inflammatory signalling and inhibit (or reduce or limit) pathological inflammation associated with IL-6 signalling.

Thus, in some embodiments antibodies (or antigen binding proteins) of the present invention which are capable of inhibiting IL-6 trans signalling are capable of inhibiting (or reducing, or limiting) pro-inflammatory signalling and/or are capable of inhibiting (or reducing, or limiting) pathological inflammation.

In some embodiments, the antibodies (or antigen binding proteins) of the invention have the ability to maintain IL-6 classical signalling (e.g. maintain IL-6 classical signalling through (or induced by or mediated by or associated with) the human membrane-bound IL-6R).

As described elsewhere herein, IL-6 classical signalling through the membrane-bound IL-6R is recognised as being anti-inflammatory and serving protective, homeostatic and regenerative functions (e.g. defence against bacterial infection). Thus, maintenance of IL-6 classical signalling by antibodies (or antigen binding proteins) of the present invention is highly advantageous. For example, such antibodies (or antigen binding proteins) should maintain or not affect (e.g. not reduce or not inhibit, or not significantly reduce or inhibit) anti-inflammatory signalling and should maintain or not affect (e.g. not reduce or not inhibit, or not significantly reduce or inhibit) immune function (e.g. defence against bacterial infection).

Thus, in some embodiments antibodies (or antigen binding proteins) of the present invention which are capable of maintaining IL-6 classical signalling, do not affect, or do not reduce, or do not inhibit, or do not prevent (or do not significantly affect, reduce, inhibit or prevent), IL-6 classical signalling. For example, such antibodies (or antigen binding proteins) do not affect, or do not reduce, or do not inhibit, or do not prevent (or do not significantly affect, reduce, inhibit or prevent) anti-inflammatory signalling and/or do not affect, or do not reduce, or do not inhibit, or do not prevent (or do not significantly affect, reduce, inhibit or prevent) immune function (e.g. beneficial immune function such as defence against bacterial infection).

Thus, in preferred embodiments antibodies (or antigen binding proteins) of the present invention have the advantageous properties of inhibiting (or reducing, or limiting) IL-6 trans signalling or the effects of IL-6 trans signalling, such as pro-inflammatory signalling and/or pathological inflammation, while maintaining (e.g. not significantly affecting, or reducing, or inhibiting, or preventing) IL-6 classical signalling or the effects of IL-6 classical signalling such as anti-inflammatory signalling and/or immune function (e.g. beneficial immune function such as defence against bacterial infection).

Appropriate methods and assays to measure IL-6 classical signalling and IL-6 trans signalling are described elsewhere herein.

As discussed elsewhere herein, some embodiments of the present invention describe antibodies (or antigen binding proteins) binding to human IL-6R, e.g. binding to human membrane-bound and soluble IL-6R, which are capable of inhibiting trans signalling through the soluble IL-6R while maintaining classical signalling through membrane-bound IL-6R.

It is surprising that the antibodies of the present invention have the ability to bind to human soluble IL-6R and human membrane-bound IL-6R, yet selectively inhibit IL-6 trans signalling. Without wishing to be bound by theory, it is believed that the antibodies of the present invention may selectively inhibit IL-6 trans signalling (e.g. inhibit trans signalling while maintaining classical signalling) by steric hindrance, and/or steric conversion, of human soluble IL-6R. In this mechanism, binding of an antibody (or antigen binding protein) of the invention to a site on the human IL-6R, for example a site adjacent to the ligand (IL-6) binding site, may act sterically to prevent (or inhibit) subsequent formation of the IL-6/soluble IL-6R/gp130 signalling complex (the hexamer signalling complex) on the cell membrane while allowing (e.g. not inhibiting or significantly inhibiting, or not affecting or significantly affecting, or maintaining) formation of the IL-6/membrane-bound IL-6R/gp130 signalling complex (the hexamer signalling complex). Alternatively, or in addition, binding of an antibody (or antigen binding protein) of the invention may lead to steric conversion of the soluble IL-6R, whereby refolding may prevent the formation of an active IL-6 trans signalling receptor complex, while fixation of the membrane-bound IL-6R in the cell membrane could stabilize the fold of the receptor and suppress steric conversion of the membrane-bound IL-6R. Thus, formation of the soluble IL-6R signalling complex (the hexamer signalling complex involving sIL-6R and believed to contain 21L-6:sIL-6 receptor and a gp130 dimer) is prevented or inhibited or reduced by antibodies (or antigen binding proteins) of the present invention, while formation of the membrane-bound IL-6 signalling complex (the hexamer signalling complex involving membrane-bound IL-6R and believed to contain two IL-6:membrane-bound IL-6R dimers and a gp130 dimer) is not affected (or not significantly affected, or not inhibited, or not significantly inhibited, or maintained) by antibodies (or antigen binding proteins) of the present invention.

In addition, the ability of antibodies (or antigen binding proteins) of the invention to specifically bind to human IL-6R and selectively inhibit IL-6 trans signalling is advantageous to prevent unwanted, off-target effects (e.g. inhibition of other signalling pathways or functions, for example involving gp130). Without wishing to be bound by theory, the ability of the antibodies (or antigen binding proteins) of the invention to bind (or specifically recognise or specifically bind to) IL-6R prevents off-target effects (e.g. inhibition of other signalling pathways such as those involving gp130) which can occur if antibodies (or antigen binding proteins) or other biologics target (e.g. block or inhibit or involve) signalling components (e.g. receptor co-activator or signal transducer components) common to multiple signalling pathways, such as gp130. For example, signalling via other IL-6 family cytokines, such as IL-11, also requires the recruitment of gp130 downstream of cytokine/receptor complex formation for downstream signalling. The exemplified antibodies of the present invention maintain (or do not inhibit or do not significantly inhibit) IL-11 signalling. Thus, the antibodies (or antigen binding proteins) of the invention have the beneficial property of maintaining other signalling pathways (i.e. other than IL-6) that require gp130 for signal transduction, e.g. IL-11.

Thus, in some embodiments, the antibodies (or antigen binding proteins) of the invention inhibit IL-6 trans signalling while maintaining IL-6 classical signalling, and maintain (or do not inhibit or do not significantly inhibit) other gp130-dependent signalling pathways.

The definitions for “maintains (or does not inhibit or does not significantly inhibit) IL-6 classical signalling” as used elsewhere herein apply mutatis mutandis to “maintain (or do not inhibit or do not significantly inhibit) other gp130-dependent signalling pathways (e.g. IL-11 signalling)”.

“Other gp130-dependent pathways” as used herein refers to, but is not limited to, signalling pathways, e.g. one or more signalling pathways, involving the cytokines: IL-11, leukaemia inhibitory factor (LIF), ciliary neurotrophic factor (CNTF), oncostatin M (OSM), cardiotrophin 1 (CT1), cardiotrophin-like cytokine (CLC), and IL-27.

Thus, in some embodiments, the antibodies (or antigen binding proteins) of the invention inhibit IL-6 trans signalling while maintaining IL-6 classical signalling and maintaining IL-11 signalling.

In some embodiments, the antibodies (or binding proteins) of the present invention bind to (or specifically bind to) human IL-6R and inhibit IL-6 trans signalling while maintaining IL-6 classical signalling, and further possess one or more, e.g. two or three, of the following properties:
i. bind to the soluble and/or (preferably “and”) the membrane-bound human IL-6R;
ii. do not bind to IL-6R (soluble and/or (preferably “and”) membrane-bound IL-6R) in some non-human species, e.g. murine (or mouse), and/or (preferably “and”) maintain (or do not inhibit or do not significantly inhibit), or do not affect IL-6 classical signalling or IL-6 trans signalling in some non-human species, e.g. through the appropriate non-human IL-6R, e.g. murine (or mouse) IL-6R;
iii. maintain (or do not inhibit or do not significantly inhibit) other gp130-dependent signalling pathways, e.g. maintain (or do not inhibit or do not significantly inhibit) IL-11 signalling.

In some embodiments, the antibodies (or binding proteins) of the present invention further possess one or both of the following properties:
iv. do not preferentially bind (or do not bind with a greater affinity or significantly greater affinity) to human IL-6/IL-6R complex (soluble and/or membrane human IL-6/IL-6R complex) as compared to human IL-6R (soluble and/or membrane-bound human IL-6R) alone;
v. do not affect (or do not significantly affect) the IL-6 buffering system (or do not reduce the level of or amount of (or do not significantly reduce the level of or amount of) free IL-6 in the extracellular space.

Methods of assessing IL-6 classical signalling and/or IL-6 trans signalling and/or IL-11 signalling (including IL-11 classical and trans signalling) may be any appropriate method and would be well-known to a person skilled in the art. Preferred methods or assays for assessing said signalling pathways are discussed herein.

In some embodiments, human IL-6 classical signalling may be assessed using a cellular model for cell proliferation (also referred to herein as a cell proliferation assay), wherein cells express (endogenously and/or through genetic engineering) both membrane-bound IL-6R and gp130 (here human membrane-bound IL-6R and gp130), and are cultured in the presence of human IL-6 cytokine. This system lacks additional growth factors to IL-6 and lacks soluble IL-6R, and thus can be used to measure (or assess or quantify) cell proliferation induced by IL-6 classical signalling only.

In preferred embodiments, to measure (or assess or quantify) human IL-6 classical signalling using a cellular model for cell proliferation, the cellular model uses a cell line expressing human gp130 and human membrane-bound IL-6R, for example a murine pre-B cell line genetically engineered to express human gp130 and human membrane-bound IL-6R (Ba/F3_gp130_IL-6R). In preferred embodiments, cells are cultured with human IL-6 cytokine (preferably at 10 ng/ml) and treated with the antibody (or antigen binding protein) of the invention, e.g. C07 and/or C09 and/or C12 and/or C19 and/or C20 (preferably at a concentration of 1-10 μg/ml, more preferably 1 or 10 μg/ml), or treated with a control with no antibody (i.e. cytokine only control). After an appropriate time period, e.g. after 48 h, the number of viable cells (and hence cell proliferation) is determined by measuring (or quantifying) the number of metabolically active cells (e.g. using Cell Titer Blue reagent). In preferred embodiments, the number of viable cells in the sample treated with a control with no antibody represents (or is set as) the basal signalling level (or 100% signalling level or value). In preferred embodiments, the number of viable cells in the sample treated with an antibody (or antigen binding protein) of the present invention is normalised to (i.e. divided by and then multiplied by 100) the number of viable cells in the sample treated with a control with no antibody. These values can then be used to measure (or assess or quantify) the ability of the antibody (or antigen binding protein) to affect IL-6 classical signalling.

Exemplary antibodies (or antigen binding proteins) of the invention show the ability to maintain (e.g. not significantly affect or not significantly reduce) human IL-6 classical signalling through the human membrane-bound IL-6R, when measured (or assessed or quantified) using a cell proliferation assay, such as the assay described above. Thus, e.g. when assessed in such assays, such antibodies (or antigen binding proteins) of the invention show the ability to maintain (e.g. not significantly affect or not significantly reduce) the level of cell proliferation induced by IL-6 classical signalling (IL-6 classical signalling through the human membrane-bound IL-6R), as compared to an appropriate control level or assay, e.g. a level of cell proliferation observed in the same assay, but in the absence of the antibody (or antigen binding protein). Thus, the proliferation observed in the absence of the antibody (or antigen binding protein) is an appropriate and exemplary control level. Exemplary maintenance of IL-6 classical signalling levels in terms of % levels of inhibition (here low % levels of inhibition) are described elsewhere herein.

In some embodiments, human IL-6 trans signalling may be assessed using a cellular model for cell proliferation (also referred to herein as a cell proliferation assay), wherein cells express (endogenously or through genetic engineering) gp130 (here human gp130), and are cultured in the presence of human soluble IL-6R and IL-6 cytokine. This system includes soluble IL-6R but lacks membrane-bound IL-6R, and lacks additional growth factors to IL-6, and thus can be used to measure (or assess or quantify) cell proliferation induced by IL-6 trans signalling only.

In preferred embodiments, to measure (or assess or quantify) human IL-6 trans signalling using a cellular model for cell proliferation, the cellular model uses a cell line expressing human gp130, for example a murine pre-B cell line genetically engineered to express human gp130 (Ba/F3_gp130). In preferred embodiments, cells are cultured with human IL-6 cytokine (preferably at a concentration of 10 ng/ml) and human soluble IL-6R (preferably at a concentration of 100 ng/ml or 200 ng/ml), or cultured with Hyper-IL-6 (Hy-IL-6, a recombinant fusion protein consisting of IL-6 and soluble IL-6R (Peters et al., J Immunol., 1998; 161(7): 3575-81)) (preferably at a concentration of 28 ng/ml), and treated with the antibody (or antigen binding protein) of the invention, e.g. C07 and/or C09 and/or C12 and/or C19 and/or C20 (preferably at a concentration of 1-10 μg/ml, more preferably 1 or 10 μg/ml), or treated with a control with no antibody (i.e. cytokine and soluble IL-6R only control). After an appropriate time period, e.g. after 48 h, the number of viable cells (and hence cell proliferation) is determined by measuring (or quantifying) the number of metabolically active cells (e.g. using Cell Titer Blue reagent). In preferred embodiments, the number of viable cells in the sample treated with a control with no antibody represents (or is set as) the basal signalling level (or 100% signalling level or value). In preferred embodiments, the number of viable cells in the sample treated with an antibody (or antigen binding protein) of the present invention is normalised to (i.e. divided by and then multiplied by 100) the number of viable cells in the sample treated with a control with no antibody. These values can then be used to measure (or assess or quantify) the ability of the antibody (or antigen binding protein) to affect IL-6 trans signalling.

Exemplary antibodies (or antigen binding proteins) of the invention show the ability to inhibit (e.g. reduce or limit, e.g. significantly reduce or limit) human IL-6 trans signalling through the human soluble IL-6R, when measured (or assessed or quantified) using a cell proliferation assay, such as the assay described above. Thus, e.g. when assessed in such assays, such antibodies (or antigen binding proteins) of the invention show the ability to inhibit (e.g. reduce or limit, e.g. significantly inhibit or reduce or limit) the level of cell proliferation induced by IL-6 trans signalling (trans signalling through the human soluble IL-6R), as compared to an appropriate control level or assay, e.g. a level of cell proliferation observed in the same assay, but in the absence of the antibody (or antigen binding protein). Thus, the proliferation observed in the absence of the antibody (or antigen binding protein) is an appropriate and exemplary control level. Exemplary inhibition of IL-6 trans signalling levels in terms of % levels of inhibition (here high % levels of inhibition) are described elsewhere herein.

In some embodiments, combined human IL-6 classical and trans signalling (also referred to herein as “IL-6 mixed signalling”) may be assessed using a cellular model for cell proliferation (also referred to herein as a cell proliferation assay), wherein cells express (endogenously and/or through genetic engineering) both membrane-bound IL-6R and gp130 (here human membrane-bound IL-6R and gp130), and are cultured in the presence of both human IL-6 cytokine and soluble IL-6R. In the body, cells are confronted with both IL-6 cytokine and soluble IL-6R, allowing combined IL-6 classical and trans signalling (mixed signalling) in cells expressing membrane-bound IL-6R. However, high expression of membrane-bound IL-6R favours classical signalling (Reeh et al. Cell Comm Signal. 2019. 17(1):46). Therefore, this system allows classical signalling (and maintenance of classical signalling) to be assessed under more physiological conditions.

In preferred embodiments, to measure (or assess or quantify) human IL-6 mixed signalling using a cellular model for cell proliferation, the cellular model uses a cell line expressing human gp130 and membrane-bound IL-6R, for example a murine pre-B cell line genetically engineered to express human gp130 and human membrane-bound IL-6R (Ba/F3_gp130_IL-6R). In preferred embodiments, cells are cultured with human IL-6 cytokine (preferably at a concentration of 10 ng/ml) and human soluble IL-6R (preferably at a concentration of 100 ng/ml or 200 ng/ml), and treated with the antibody (or antigen binding protein) of the invention, e.g. C07 and/or C09 and/or C12 and/or C19 and/or C20 (preferably at a concentration of 1-10 μg/ml, more preferably 1 or 10 μg/ml), or treated with a control with no antibody (i.e. cytokine and soluble IL-6R only control). After an appropriate time period, e.g. after 48 h, the number of viable cells (and hence cell proliferation) is determined by measuring (or quantifying) the number of metabolically active cells (e.g. using Cell Titer Blue reagent). In preferred embodiments, the number of viable cells in the sample treated with a control with no antibody represents (or is set as) the basal signalling level (or 100% signalling level or value). In preferred embodiments, the number of viable cells in the sample treated with an antibody (or antigen binding protein) of the present invention is normalised to (i.e. divided by and then multiplied by 100) the number of viable cells in the sample treated with a control with no antibody. These values can then be used to measure (or assess or quantify) the ability of the antibody (or antigen binding protein) to affect IL-6 classical signalling.

Exemplary antibodies (or antigen binding proteins) of the invention show the ability to maintain (e.g. not significantly affect or not significantly reduce) human IL-6 classical signalling through the human membrane-bound IL-6R, when measured (or assessed or quantified) using a mixed signalling cell proliferation assay, such as the assay described above. Thus, e.g. when assessed in such assays, such antibodies (or antigen binding proteins) of the invention show the ability to maintain (e.g. not significantly affect or not significantly reduce) the level of cell proliferation induced by IL-6 classical signalling (IL-6 classical signalling through the human membrane-bound IL-6R), as compared to an appropriate control level or assay, e.g. a level of cell proliferation observed in the same assay, but in the absence of the antibody (or antigen binding protein). Thus, the proliferation observed in the absence of the antibody (or antigen binding protein) is an appropriate and exemplary control level. Exemplary maintenance of IL-6 classical signalling levels in terms of % levels of inhibition (here low % levels of inhibition) are described elsewhere herein.

In preferred embodiments, human IL-11 classical, trans and mixed signalling can be measured (or assessed or quantified) using the above cellular assays for IL-6 classical, trans and mixed signalling, however replacing Ba/F3_gp130_IL-6R cells with Ba/F3_gp130_IL-11R cells (expressing human gp130 and human membrane-bound IL-11R), and replacing human IL-6 cytokine with human IL-11 cytokine (preferably at 10 ng/ml) and human soluble IL-6R with human soluble IL-11R (preferably at a concentration of 200 ng/ml), and treating with antibodies (or antigen binding proteins) of the invention at a preferred concentration of 10 μg/ml).

Exemplary antibodies (or antigen binding proteins) of the invention show the ability to maintain (e.g. not significantly affect or not significantly reduce or do not inhibit or do not significantly inhibit) human IL-11 classical, trans and mixed signalling, and the assessment of such assays as described for “maintains IL-6 classical signalling” above applies mutatis mutandis to this aspect of the invention.

In preferred embodiments, murine IL-6 classical, trans and mixed signalling can be measured (or assessed or quantified) using the above cellular assays for IL-6 classical, trans and mixed signalling, however replacing Ba/F3_gp130_IL-6R cells with Ba/F3_gp130_muIL-6R cells (expressing human gp130 and murine membrane-bound IL-6R), and replacing human IL-6 cytokine with murine IL-6 cytokine (preferably at 10 ng/ml) and human soluble IL-6R with murine soluble IL-6R (preferably at a concentration of 200 ng/ml), and treating with antibodies (or antigen binding proteins) of the invention at a preferred concentration of 10 μg/ml). Notably, murine IL-6R is capable of forming an active receptor complex with human gp130.

Exemplary antibodies (or antigen binding proteins) of the invention show the ability to maintain (e.g. not significantly affect or not significantly reduce, or do not inhibit or do not significantly inhibit) murine IL-6 classical, trans and mixed signalling, and the assessment of such assays as described for “maintains IL-6 classical signalling” above applies mutatis mutandis to this aspect of the invention.

In some embodiments, IL-3 signalling may be assessed using a cellular model for cell proliferation (also referred to herein as a cell proliferation assay), wherein cells express (endogenously and/or through genetic engineering) membrane-bound IL-3R (here murine membrane-bound IL-3R), and are cultured in the presence of murine IL-3 cytokine. This system lacks additional growth factors to IL-3, and thus can be used to measure (or quantify) cell proliferation induced by IL-3 signalling only, wherein antibodies (or binding proteins) of the present invention can be assessed for IL-6 independent effects on cell proliferation.

In preferred embodiments, to measure (or assess or quantify) IL-3 signalling using a cellular model for cell proliferation, the cellular model uses a cell line expressing murine membrane-bound IL-3R, for example a murine pre-B cell line (Ba/F3) expressing murine membrane-bound IL-3R, e.g. endogenously expressing murine membrane-bound IL-3R. In preferred embodiments, cells are cultured with recombinant murine IL-3 cytokine (preferably at 10 ng/ml) and treated with the antibody (or antigen binding protein) of the invention, e.g. C07 and/or C09 and/or C12 and/or C19 and/or C20 (preferably at a concentration of 1-10 μg/ml, more preferably 1 or 10 μg/ml), or treated with a control with no antibody (i.e. cytokine (IL-3) only control). After an appropriate time period, e.g. after 48 h, the number of viable cells (and hence cell proliferation) is determined by measuring (or quantifying) the number of metabolically active cells (e.g. using Cell Titer Blue reagent). In preferred embodiments, the number of viable cells in the sample treated with a control with no antibody represents (or is set as) the basal signalling level (or 100% signalling level or value). In preferred embodiments, the number of viable cells in the sample treated with an antibody (or antigen binding protein) of the present invention is normalised to (i.e. divided by and then multiplied by 100) the number of viable cells in the sample treated with a control with no antibody. These values can then be used to measure (or assess or quantify) the ability of the antibody (or antigen binding protein) to affect IL-3 signalling, or non-IL-6 signalling, or IL-6 independent signalling. In other words, can be used to give an indication of the specificity of the antibody (or antigen binding protein) for IL-6R.

Preferred exemplary antibodies (or antigen binding proteins) of the invention show no effect or an insignificant effect, on IL-3 signalling through the murine membrane-bound IL-3R, e.g. when measured (or assessed or quantified) using a cell proliferation assay, such as the assay described above. Thus, e.g. when assessed in such assays, such antibodies (or antigen binding proteins) of the invention show no effect or an insignificant effect on the level of cell proliferation induced by IL-3 signalling (IL-3 signalling through the murine membrane-bound IL-3R), as compared to an appropriate control level or assay, e.g. a level of cell proliferation observed in the same assay, but in the absence of the antibody (or antigen binding protein). Thus, the proliferation observed in the absence of the antibody (or antigen binding protein) is an appropriate and exemplary control level.

In some embodiments, IL-6 classical signalling may be assessed using a cellular model for intracellular IL-6 signalling (also referred to herein as an intracellular IL-6 signalling assay or an intracellular signalling assay), wherein cells express (endogenously and/or through genetic engineering) both membrane-bound IL-6R and gp130 (here human membrane-bound IL-6R and gp130), and are cultured in the presence of human IL-6 cytokine. This system lacks additional growth factors to IL-6 and lacks soluble IL-6R, and thus can be used to measure (or assess or quantify) intracellular signalling induced by IL-6 classical signalling only.

In preferred embodiments, to measure (or assess or quantify) IL-6 classical signalling using a cellular model for intracellular IL-6 signalling, the cellular model uses a cell line expressing human gp130 and membrane-bound IL-6R, for example HEK293 cells endogenously expressing human gp130 and genetically engineered to express human membrane-bound IL-6R (HEK293_IL-6R). In preferred embodiments, cells are cultured with human IL-6 cytokine (preferably at 10 ng/ml) and treated with the antibody (or antigen binding protein) of the invention, e.g. C07 and/or C09 and/or C12 and/or C19 and/or C20 (preferably at a concentration of 1-10 μg/ml, more preferably 1 μg/ml), or treated with a control with no antibody (i.e. cytokine only control). After an appropriate time period, e.g. after 48 h, intracellular IL-6 signalling is measured (or quantified) by measuring the level of (or amount of or fluorescent intensity of) STAT3 phosphorylated on Y705, e.g. using flow cytometry (e.g. using fluorophore-coupled antibodies which bind to (or specifically bind to or detect) STAT3 phosphorylated on Y705). In preferred embodiments, the level of (or amount of or fluorescent intensity of) STAT3 phosphorylated on Y705 in the sample treated with a control with no antibody represents (or is set as) the basal signalling level (or 100% signalling level or value). In preferred embodiments, the level of (or amount of or fluorescent intensity of) STAT3 phosphorylated on Y705 in the sample treated with an antibody (or antigen binding protein) of the present invention is normalised to (i.e. divided by and then multiplied by 100) the level of (or amount of or fluorescent intensity of) STAT3 phosphorylated on Y705 in the sample treated with a control with no antibody. These values can then be used to measure (or assess or quantify) the ability of the antibody (or antigen binding protein) to affect IL-6 classical signalling.

Exemplary antibodies (or antigen binding proteins) of the invention show the ability to maintain (e.g. not significantly affect or not significantly reduce) IL-6 classical signalling through the human membrane-bound IL-6R, when measured (or assessed or quantified) using an intracellular IL-6 signalling assay, such as the assay described above. Thus, e.g. when assessed in such assays, such antibodies (or antigen binding proteins) of the invention show the ability to maintain (e.g. not significantly affect or not significantly reduce) the level of intracellular IL-6 signalling induced by IL-6 classical signalling (IL-6 classical signalling through the human membrane-bound IL-6R), as compared to an appropriate control level or assay, e.g. a level of intracellular IL-6 signalling observed in the same assay, but in the absence of the antibody (or antigen binding protein). Thus, the intracellular IL-6 signalling observed in the absence of the antibody (or antigen binding protein) is an appropriate and exemplary control level. Exemplary maintenance of IL-6 classical signalling levels in terms of % levels of inhibition (here low % levels of inhibition) are described elsewhere herein.

In some embodiments, IL-6 trans signalling may be assessed using a cellular model for intracellular IL-6 signalling (also referred to herein as an intracellular IL-6 signalling assay or an intracellular signalling assay), wherein cells express (endogenously or through genetic engineering) gp130 (here human gp130), and are cultured in the presence of human soluble IL-6R and IL-6 cytokine. This system includes soluble IL-6R but lacks membrane-bound IL-6R, and lacks additional growth factors to IL-6, and thus can be used to measure (or assess or quantify) intracellular signalling induced by IL-6 trans signalling only.

In preferred embodiments, to measure (or assess or quantify) IL-6 trans signalling using a cellular model for intracellular IL-6 signalling, the cellular model uses a cell line expressing human gp130, for example HEK293 cells endogenously expressing human gp130 (HEK293). In preferred embodiments, cells are cultured with human IL-6 cytokine (preferably at 10 ng/ml) and human soluble IL-6R (preferably at a concentration of 100 ng/ml or 200 ng/ml), and treated with the antibody (or antigen binding protein) of the invention, e.g. C07 and/or C09 and/or C12 and/or C19 and/or C20 (preferably at a concentration of 1-10 μg/ml, more preferably 1 μg/ml), or treated with a control with no antibody (i.e. cytokine and soluble IL-6R only control). After an appropriate time period, e.g. after 48 h, intracellular IL-6 signalling is measured (or quantified) by measuring the level of (or amount of or fluorescent intensity of) STAT3 phosphorylated on Y705, e.g. using flow cytometry (e.g. using fluorophore-coupled antibodies which bind to (or specifically bind to or detect) STAT3 phosphorylated on Y705). In preferred embodiments, the level of (or amount of or fluorescent intensity of) STAT3 phosphorylated on Y705 in the sample treated with a control with no antibody represents (or is set as) the basal signalling level (or 100% signalling level or value). In preferred embodiments, the level of (or amount of or fluorescent intensity of) STAT3 phosphorylated on Y705 in the sample treated with an antibody (or antigen binding protein) of the present invention is normalised to (i.e. divided by and then multiplied by 100) the level of (or amount of or fluorescent intensity of) STAT3 phosphorylated on Y705 in the sample treated with a control with no antibody. These values can then be used to measure (or assess or quantify) the ability of the antibody (or antigen binding protein) to affect IL-6 trans signalling.

Exemplary antibodies (or antigen binding proteins) of the invention show the ability to inhibit (e.g. reduce or limit, e.g. significantly reduce or limit) IL-6 trans signalling through the human soluble IL-6R, when measured (or assessed or quantified) using an intracellular IL-6 signalling assay, such as the assay described above. Thus, e.g. when assessed in such assays, such antibodies (or antigen binding proteins) of the invention show the ability to inhibit (e.g. reduce or limit, e.g. significantly inhibit or reduce or limit) the level of intracellular IL-6 signalling induced by IL-6 trans signalling (trans signalling through the human soluble IL-6R), as compared to an appropriate control level or assay, e.g. a level of intracellular IL-6 signalling observed in the same assay, but in the absence of the antibody (or antigen binding protein). Thus, the intracellular IL-6 signalling observed in the absence of the antibody (or antigen binding protein) is an appropriate and exemplary control level. Exemplary inhibition of IL-6 trans signalling levels in terms of % levels of inhibition (here high % levels of inhibition) are described elsewhere herein.

In some embodiments, combined IL-6 classical and trans signalling (IL-6 mixed signalling) may be assessed using a cellular model for intracellular IL-6 signalling (also referred to herein as an intracellular IL-6 signalling assay or an intracellular signalling assay), wherein cells express (endogenously and/or through genetic engineering) both membrane-bound IL-6R and gp130 (here human membrane-bound IL-6R and gp130), and are cultured in the presence of both human IL-6 cytokine and soluble IL-6R. As previously described, this system allows classical signalling to be monitored under physiological conditions.

In preferred embodiments, to measure (or assess or quantify) IL-6 mixed signalling using a cellular model for intracellular IL-6 signalling, the cellular model uses a cell line expressing human gp130 and membrane-bound IL-6R, for example HEK293 cells endogenously expressing human gp130 and genetically engineered to express membrane-bound IL-6R (HEK293_IL-6R). In preferred embodiments, cells are cultured with human IL-6 cytokine (preferably at 10 ng/ml) and human soluble IL-6R (preferably at a concentration of 100 ng/ml or 200 ng/ml), and treated with the antibody (or antigen binding protein) of the invention, e.g. C07 and/or C09 and/or C12 and/or C19 and/or C20 (preferably at a concentration of 1-10 μg/ml, more preferably 1 μg/ml), or treated with a control with no antibody (i.e. cytokine and soluble IL-6R only control). After an appropriate time period, e.g. after 48 h, intracellular IL-6 signalling is measured (or quantified) by measuring the level of (or amount of or fluorescent intensity of) STAT3 phosphorylated on Y705, e.g. using flow cytometry (e.g. using fluorophore-coupled antibodies which bind to (or specifically bind to or detect) STAT3 phosphorylated on Y705). In preferred embodiments, the level of (or amount of or fluorescent intensity of) STAT3 phosphorylated on Y705 in the sample treated with a control with no antibody represents (or is set as) the basal signalling level (or 100% signalling level or value). In preferred embodiments, the level of (or amount of or fluorescent intensity of) STAT3 phosphorylated on Y705 in the sample treated with an antibody (or antigen binding protein) of the present invention is normalised to (i.e. divided by and then multiplied by 100) the level of (or amount of or fluorescent intensity of) STAT3 phosphorylated on Y705 in the sample treated with a control with no antibody. These values can then be used to measure (or assess or quantify) the ability of the antibody (or antigen binding protein) to affect IL-6 classical signalling.

Exemplary antibodies (or antigen binding proteins) of the invention show the ability to maintain (e.g. not significantly affect or not significantly reduce) IL-6 classical signalling through the human membrane-bound IL-6R, when measured (or assessed or quantified) using a mixed signalling intracellular IL-6 signalling assay, such as the assay described above. Thus, e.g. when assessed in such assays, such antibodies (or antigen binding proteins) of the invention show the ability to maintain (e.g. not significantly affect or not significantly reduce) the level of intracellular IL-6 signalling induced by IL-6 classical signalling (IL-6 classical signalling through the human membrane-bound IL-6R), as compared to an appropriate control level or assay, e.g. a level of intracellular IL-6 signalling observed in the same assay, but in the absence of the antibody (or antigen binding protein). Thus, the intracellular IL-6 signalling observed in the absence of the antibody (or antigen binding protein) is an appropriate and exemplary control level. Exemplary maintenance of IL-6 classical signalling levels in terms of % levels of inhibition (here low % levels of inhibition) are described elsewhere herein.

Thus, in some embodiments, one or more of IL-6 classical signalling, IL-6 trans signalling and IL-6 mixed signalling can be separately measured (or assessed or quantified) using appropriate cellular models expressing specific combinations of receptors (e.g. gp130 and/or membrane-bound IL-6R) and culturing said cellular models with the appropriate cytokine and/or soluble receptor (e.g. IL-6 and/or soluble IL-6R) to stimulate either IL-6 classical, trans and/or mixed signalling pathways. In some embodiments, IL-6 classical, trans and mixed signalling are measured (or assessed or quantified) using downstream IL-6 signalling events (e.g. an intracellular cell signalling assay for example to measure STAT3 phosphorylation on Y705) and/or downstream cellular responses to IL-6 signalling (e.g. cell proliferation) which are common to both IL-6 classical, trans and mixed signalling pathways.

As described above, in one aspect of the invention, antibodies (or antigen binding proteins) of the invention bind to human IL-6R and inhibit IL-6 trans signalling (IL-6 mediated trans signalling) through the human soluble IL-6R while maintaining IL-6 classical signalling (IL-6 mediated classical signalling) through the human membrane-bound IL-6R.

In some embodiments, the ability of an antibody or antigen binding protein of the invention to inhibit IL-6 trans signalling while maintaining IL-6 classical signalling can be determined by a ratio of classical signalling to trans signalling (also referred to herein as “classical:trans signalling ratio”).

In some embodiments, to determine the classical:trans signalling ratio, the level of (or amount, or measurable amount of) active (or residual) IL-6 classical signalling and IL-6 trans signalling are separately determined after treatment with an antibody (or antigen binding protein) of the invention as compared to (or normalised to or relative to) an appropriate control, for example a control with no antibody, or as compared to a control with an antibody that does not bind IL-6R, wherein the level of (or amount, or measurable amount of) both IL-6 classical signalling and IL-6 trans signalling observed with said control represents (or is set as) the basal signalling level (or 100% signalling level or value) for each pathway (i.e. the level of IL-6 classical signalling and IL-6 trans signalling are individually set as 100% signalling after treatment with said control). The classical:trans signalling ratio can then be calculated or determined for each antibody (or antigen binding protein), after treatment with said antibody (or binding protein), by dividing the residual level of (or amount, or measurable amount of) IL-6 classical signalling (normalised to said control) by the residual level of (or amount, or measurable amount of) IL-6 trans signalling (normalised to said control). Thus, in some embodiments, the classical:trans signalling ratio discussed elsewhere herein are normalised to (or relative to or compared to) the basal signalling level observed with an appropriate control, e.g. a control with no antibody, or a control with an antibody that does not bind IL-6R, preferably normalised to (or relative to or compared to) the basal signalling level observed with a control with no antibody.

Thus, after treatment with an antibody (or antigen binding protein) of the invention, a classical:trans signalling ratio of 1.0 indicates a similar level or residual level of IL-6 classical signalling and IL-6 trans signalling, indicating a similar level of inhibition (or lack thereof) of both pathways by said antibodies (or binding proteins). A ratio of >1.0 indicates a higher residual level of IL-6 classical signalling compared to IL-6 trans signalling, thus indicating a greater or increased inhibition of IL-6 trans signalling compared to IL-6 classical signalling by said antibodies (or binding proteins). A ratio of <1.0 indicates a higher residual level of IL-6 trans signalling compared to IL-6 classical signalling, thus indicating a greater or increased inhibition of IL-6 classical signalling compared to IL-6 trans signalling by said antibodies (or binding proteins).

Thus, in some embodiments, antibodies (or antigen binding proteins) of the invention that inhibit IL-6 trans signalling while maintaining IL-6 classical signalling are defined by a classical:trans signalling ratio of greater than 1.0.

In preferred embodiments, antibodies (or antigen binding proteins) of the invention that inhibit IL-6 trans signalling while maintaining IL-6 classical signalling are defined by a classical:trans signalling ratio of at least 1.1, 1.2, 1.3, 1.4, 1.5, 1.6, 1.7, 1.8, 1.9, 2.0, 2.2, 2.5, 3.0, 3.5, 4.0, 4.5, 5.0, 5.5, 6.0, 6.5, 7.0, 7.5, 8.0, 8.5, 9.0, 9.5, 10.0, 11.0, 12.0, 14.0, 15.0, 16.0, 18.0, 20.0, 25.0, 30.0, 40.0, 50.0, 75.0, 100, 150, 200, 250, 300, 350, 400, 450 or 500.

In some embodiments, antibodies (or antigen binding proteins) that inhibit IL-6 trans signalling while maintaining IL-6 classical signalling can be determined by performing an appropriate test (or assay) to determine (or quantify) the level of (or amount of or residual) IL-6 trans signalling through the soluble IL-6R and an appropriate test (or assay) to determine (or quantify) the level of (or amount of or residual) IL-6 classical signalling through the membrane-bound IL-6R. Suitable assays are described elsewhere herein, for example assays that measure IL-6 classical signalling, IL-6 trans signalling and IL-6 mixed (combined) signalling are described herein and can be used, for example in some embodiments assays that measure IL-6 classical signalling and IL-6 trans signalling as described herein can be used (e.g. a cell proliferation or an intracellular IL-6 signalling assay, e.g. as described herein). However any assays measuring IL-6 trans signalling and IL-6 classical signalling are suitable for (and can be used) to determine the classical:trans signalling ratio as defined herein, although cell-based assays are preferred.

In preferred embodiments, to determine the classical:trans signalling ratio, values for IL-6 classical signalling and IL-6 trans signalling are determined using the same assay (e.g. a cell proliferation or an intracellular IL-6 signalling assay, e.g. as described herein).

In addition, typically, the same concentration of antibody (binding protein) is used in both such assays (i.e. the same concentration of antibody or binding protein is used in the test to determine the level of inhibition of IL-6 trans signalling and the level of inhibition of IL-6 classical signalling). Thus, in some embodiments, antibodies (or binding proteins) that inhibit IL-6 trans signalling while maintaining IL-6 classical signalling are determined when the antibody (or binding protein) is used at a concentration of 0.01, 0.1, 0.3, 0.5, 0.7, 1, 5 or 10 μg/ml, preferably 1-10 μg/ml. Thus, in preferred embodiments the above classical:trans signalling ratios apply when the antibodies (or binding proteins) are used at a concentration of 1 or 10 μg/ml, for example 10 μg/ml.

Alternatively, the classical:trans signalling ratio of an antibody (or antigen binding protein) of the invention can be determined by calculating the IC50 value (i.e. the concentration of antibody (or antigen binding protein) required to inhibit a biological process, for example cell proliferation, by a half (50%)) of IL-6 classical signalling and IL-6 trans signalling, using the % inhibitions of IL-6 classical signalling and % inhibitions of IL-6 trans signalling as described elsewhere herein, over an increasing concentration range of antibody (e.g. 0.01, 0.1, 0.3, 0.5, 0.7, 1, 5 and 10 μg/ml). In this aspect, the classical:trans signalling IC50 ratio (as referred to elsewhere herein) is calculated by dividing the IC50 of IL-6 classical signalling by the IC50 of IL-6 trans signalling. The skilled person will be familiar with calculating IC50 values based on the % inhibitions of IL-6 classical signalling and IL-6 trans signalling as defined elsewhere herein.

In some embodiments, antibodies (or antigen binding proteins) of the invention that inhibit IL-6 trans signalling while maintaining IL-6 classical signalling are defined by a classical:trans signalling IC50 ratio of at least 1.0, at least 1.5, at least 2.0, at least 3.0, at least 4.0, at least 5.0, at least 6.0, at least 7.0, at least 8.0, at least 9.0, at least 10.0, at least 11.0, at least 12.0, at least 13.0, at least 14.0, or at least 15.0, preferably when the IC50 is calculated using a cell-based assay, preferably a cell-based proliferation assay.

In preferred embodiments, antibodies (or antigen binding proteins) of the invention that inhibit IL-6 trans signalling while maintaining IL-6 classical signalling are defined by a classical:trans signalling IC50 ratio of at least 1.5, at least 3.0, at least 6.0, at least 9.0, at least 10.0, at least 15.0, preferably when the IC50 is calculated using a cell-based assay, preferably a cell-based proliferation assay.

In some embodiments, the above classical:trans signalling ratios apply when the antibody is a monoclonal antibody (e.g. as opposed to a polyclonal antibody). In preferred embodiments, the above ratios apply when the antibody is a monoclonal full length (whole) antibody, e.g. a chimeric full length (whole) antibody, or a Fab fragment, most preferably a monoclonal full length (whole) antibody, e.g. a chimeric full length (whole) antibody.

For example, the exemplified antibodies of the invention have been shown to have a classical:trans signalling ratio of at least 3.0, 3.5, 4.0, 4.5, 5.0, 5.5, 6.0, 6.5, 7.0, 7.5, 8.0, 8.5, 9.0, 9.5, 10.0, 11.0, or 12.0, when assessed as chimeric full length (whole) antibodies using a cell proliferation assay at a concentration of 10 μg/ml.

For example, the exemplified antibodies of the invention have a classical:trans signalling IC50 ratio of at least 1.5, at least 8.0, at least 10.0, at least 15.0, when using a cell-based proliferation assay.

As described above, in one aspect of the invention, antibodies (or antigen binding proteins) of the invention bind to human IL-6R and inhibit IL-6 trans signalling through the human soluble IL-6R while maintaining IL-6 classical signalling through the human membrane-bound IL-6R.

The inhibition of IL-6 trans signalling (or equivalent terms) as referred to herein, by an antibody (or antigen binding protein) of the invention, includes any measurable or significant inhibition, reduction, limitation, prevention, or block, etc., more preferably a statistically significant inhibition, reduction, limitation, prevention, or block, etc., of IL-6 trans signalling (e.g. as compared to an appropriate control, e.g. a control with no antibody, or as compared to a control with an antibody that does not bind IL-6R).

In some embodiments, the level of inhibition (or amount of inhibition) of IL-6 trans signalling observed with (or caused by or elicited by) a control, e.g. a control with no antibody, or a control antibody that does not bind IL-6R represents (or is set as) the zero inhibition level (or zero inhibition value or 0% inhibition level or value). Thus, in some embodiments, the % inhibitions of IL-6 trans signalling discussed elsewhere herein are normalised to (or relative to or compared to) the inhibition observed with (or caused by or elicited by) a control, e.g. a control with no antibody or a control antibody that does not bind IL-6R, preferably normalised to (or relative to or compared to) the inhibition observed with a control with no antibody.

In some embodiments, the inhibition of IL-6 trans signalling by an antibody (or antigen binding protein) of the invention is an inhibition of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100%, e.g. when compared to an appropriate control as discussed herein.

In preferred embodiments, the inhibition of IL-6 trans signalling by an antibody (or antigen binding protein) of the invention is an inhibition of at least 25%, at least 30%, at least 35%, at least 40%, at least 45%, at least 50%, at least 55%, at least 60%, at least 65%, at least 70%, at least 75%, at least 80%, at least 85%, at least 88%, at least 90%, at least 92%, at least 94%, at least 95%, at least 96%, at least 98%, or 100%, e.g. when compared to an appropriate control as discussed herein.

In some embodiments, antibodies (or antigen binding proteins) that inhibit IL-6 trans signalling can be determined by performing an appropriate test (or assay) to determine (or quantify) the level of (or amount of or residual) IL-6 trans signalling through the soluble IL-6R. Suitable assays for measuring IL-6 trans signalling are described elsewhere herein and can be used. For example, in preferred embodiments, levels or values for IL-6 trans signalling (or the inhibition of IL-6 trans signalling) are determined using a cell proliferation or intracellular IL-6 signalling assay, e.g. as described herein. However, any assay measuring IL-6 trans signalling is suitable for (and can be used) to determine the level (or amount) of inhibition of IL-6 trans signalling as defined herein, although cell-based assays are preferred.

In addition, an appropriate concentration of antibody (binding protein) is used in such assays. Thus, in some embodiments, antibodies (or binding proteins) that inhibit IL-6 trans signalling are determined when the antibody (or binding protein) is used at a concentration of 0.01, 0.1, 0.3, 0.5, 0.7, 1, 5 or 10 μg/ml, preferably 1-10 μg/ml. Thus, in preferred embodiments the above levels or values for the inhibition of IL-6 trans signalling apply when the antibodies (or binding proteins) are used at a concentration of 1 or 10 μg/ml, for example 10 μg/ml.

Alternatively, the inhibition of IL-6 trans signalling (or equivalent terms) as referred to herein, by an antibody (or antigen binding protein) of the invention, can be determined by calculating the IC50 value (i.e. the concentration of antibody (or antigen binding protein) required to inhibit a biological process, for example cell proliferation, by a half (50%)) of IL-6 trans signalling using the % inhibitions of IL-6 trans signalling over an increasing concentration range of antibody (e.g. 0.01, 0.1, 0.3, 0.5, 0.7, 1, 5 and 10 μg/ml of antibody) as defined elsewhere herein. The skilled person will be familiar with calculating IC50 values based on the % inhibitions of IL-6 trans signalling as defined elsewhere herein.

In some embodiments, the inhibition of IL-6 trans signalling by an antibody (or antigen binding protein) of the invention is defined by an IC50 of IL-6 trans signalling of no more than (or less than) 50 nM, no more than 45 nM, no more than 40 nM, no more than 35 nM, no more than 30 nM, no more than 25 nM, no more than 20 nM, no more than 15 nM, no more 35 than 10 nM, no more than 5 nM, no more than 4 nM, no more than 3 nM, no more than 2 nM, no more than 1 nM, or no more than 0.5 nM, preferably when the IC50 is calculated using a cell-based assay, preferably a cell-based proliferation assay, e.g. as described herein.

In preferred embodiments, the inhibition of IL-6 trans signalling by an antibody (or antigen binding protein) of the invention is defined by an IC50 of IL-6 trans signalling of no more than (or less than) 35 nM, no more than 30 nM, no more than 25 nM, no more than 20 nM, no more than 15 nM, no more than 10 nM, no more than 8 nM, no more than 6 nM, no more than 5 nM, no more than 4 nM, no more than 3.5 nM, no more than 3 nM, no more than 2 nM, no more than 1.5 nM, no more than 1 nM, or no more than 0.5 nM, preferably when the IC50 is calculated using a cell-based assay, preferably a cell-based proliferation assay, e.g. as described herein.

Alternatively, in some embodiments, the inhibition of IL-6 trans signalling by an antibody (or antigen binding protein) of the invention is defined by an IC50 of IL-6 trans signalling of no more than (or less than) 5500 ng/ml, no more than 5000 ng/ml, no more than 4500 ng/ml, no more than 4000 ng/ml, no more than 3500 ng/ml, no more than 3000 ng/ml, no more than 2500 ng/ml, no more than 2000 ng/ml, no more than 1500 ng/ml, no more than 1000 ng/ml, no more than 500 ng/ml, no more than 400 ng/ml, no more than 300 ng/ml, no more than 200 ng/ml, no more than 150 ng/ml, no more than 100 ng/ml, or no more than 50 ng/ml, preferably when the IC50 is calculated using a cell-based assay, preferably a cell-based proliferation assay, e.g. as described herein.

In preferred embodiments, the inhibition of IL-6 trans signalling by an antibody (or antigen binding protein) of the invention is an IC50 of IL-6 trans signalling of no more than (or less than) 1300 ng/ml, no more than 900 ng/ml, no more than 800 ng/ml, no more than 700 ng/ml, no more than 600 ng/ml, no more than 500 ng/ml, no more than 400 ng/ml, no more than 350 ng/ml, or no more than 300 ng/ml, no more than 250 ng/ml, no more than 200 ng/ml, or no more than 100 ng/ml, preferably when the IC50 is calculated using a cell-based assay, preferably a cell-based proliferation assay, e.g. as described herein.

In some embodiments, the above levels or values for the inhibition of IL-6 trans signalling apply when the antibody is a monoclonal antibody (e.g. as opposed to a polyclonal antibody). In preferred embodiments, the above levels or values apply when the antibody is a monoclonal full length (whole) antibody, e.g. a chimeric full length (whole) antibody, or a Fab fragment, most preferably a monoclonal full length (whole) antibody, e.g. a chimeric full length (whole) antibody.

For example, the exemplified antibodies of the invention have been shown to have an ability to inhibit IL-6 trans signalling by at least 70%, 75%, 80%, 85%, 88%, or 90%, when assessed as chimeric full length (whole) antibodies using a cell proliferation assay at a concentration of 10 μg/ml.

For example, the exemplified anti-IL-6R IgG antibodies of the invention have an IC50 of IL-6 trans signalling of no more than (or less than) 2 nM (304 ng/ml), 3.5 nM (531 ng/ml), 4.3 nM (644 ng/ml), 5 nM (757 ng/ml), 5.8 nM (863 ng/ml), 8 nM (1214 ng/ml), 28 nM (4130 ng/ml), or 35 nM (5260 ng/ml), preferably determined from a cell-based assay, e.g. as described herein.

For example, the exemplified anti-IL-6R Fab fragment of the invention has an IC50 of IL-6 trans signalling of no more than (or less than) 1.9 nM (98 ng/ml) or 3.2 nM (159 ng/ml), preferably determined from a cell-based assay, e.g. as described herein.

Viewed alternatively, as a readout for inhibition of IL-6 trans signalling, the exemplified anti-IL-6R IgG antibodies of the invention reduce (or inhibit) cell proliferation by 50% at a concentration of no more than 2 nM (304 ng/ml), 3.5 nM (531 ng/ml), 4.3 nM (644 ng/ml), 5 nM (757 ng/ml), 5.8 nM (863 ng/ml), 8 nM (1214 ng/ml), 28 nM (4130 ng/ml), or 35 nM (5260 ng/ml), preferably in a cell-based assay, e.g. as described herein.

Viewed alternatively, as a readout for inhibition of IL-6 trans signalling, the exemplified anti-IL-6R Fab fragment of the invention reduces (or inhibits) cell proliferation by 50% at a concentration of no more than 1.9 nM (98 ng/ml) or 3.2 nM (159 ng/ml), preferably in a cell-based assay, e.g. as described herein.

Thus, in some embodiments, the antibodies (or antigen binding proteins) of the present invention have the ability to inhibit IL-6 trans signalling, as defined elsewhere herein, at a concentration of 0.3 μg/ml, 0.5 μg/ml, 0.7 μg/ml, 1 μg/ml, or 10 μg/ml, preferably at a concentration of 10 μg/ml.

As described above, in this aspect of the invention, antibodies (or antigen binding proteins) of the invention bind to human IL-6R and inhibit IL-6 trans signalling through the human soluble IL-6R while maintaining IL-6 classical signalling through the human membrane-bound IL-6R.

The maintenance of IL-6 classical signalling (or equivalent terms) as referred to herein, by an antibody (or antigen binding protein) of the invention, includes no affect or change (or no measurable or significant, e.g. statistically significant, affect or change) on IL-6 classical signalling, for example no reduction, inhibition or prevention, e.g. no measurable or significant (e.g. statistically significant) reduction, inhibition or prevention, of IL-6 classical signalling (e.g. as compared to an appropriate control, e.g. a control with no antibody, or as compared to a control with an antibody that does not bind IL-6R). Viewed another way, the maintenance of IL-6 classical signalling (or equivalent terms) as referred to herein, by an antibody (or antigen binding protein) of the invention, is an inhibition of IL-6 classical signalling substantially the same as (or not significantly different from or analogous to or comparable to) the inhibition of IL-6 classical signalling observed with (or caused by or elicited by) an appropriate control, e.g. a control with no antibody or a control with an antibody that does not bind IL-6R, preferably a control with no antibody.

Thus, preferred antibodies or antigen binding proteins of the invention do not affect, inhibit, reduce, prevent or block, e.g. do not significantly (e.g. statistically significantly) affect, inhibit, reduce, prevent or block, IL-6 classical signalling. Thus, alternatively viewed, the present invention provides an antibody (or antigen binding protein), which binds to human IL-6R and inhibits IL-6 trans signalling through the human soluble IL-6R without significantly inhibiting IL-6 classical signalling through the human membrane-bound IL-6R. Discussion of various features of the antibodies of other aspects of the invention and preferred embodiments apply mutatis mutandis to this aspect of the invention.

In some embodiments, the level of inhibition (or amount of inhibition) of IL-6 classical signalling observed with (or caused by or elicited by) an appropriate control, e.g. a control with no antibody represents (or is set as) the zero inhibition level (or zero inhibition value or 0% inhibition level or value). Thus, in some embodiments, the % inhibitions of IL-6 classical signalling discussed elsewhere herein are normalised to (or relative to or compared to) the inhibition observed with (or caused by or elicited by) an appropriate control, e.g. a control with no antibody or a control antibody that does not bind IL-6R, preferably normalised to (or relative to or compared to) the inhibition observed with a control with no antibody.

In some embodiments, maintenance of IL-6 classical signalling is an inhibition of no more than 30%, no more than 25%, no more than 20%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, no more than 1% or by 0%.

In some embodiments, maintenance of IL-6 classical signalling is an inhibition of less than 30%, 25%, 20%, 15%, 14%, 13%, 12%, 11%, 10%, 9%, 8%, 7%, 6%, 5%, 4%, 3%, 2%, 1% or by 0%.

In some preferred embodiments, antibodies (or antigen binding proteins) of the present invention cause (or elicit) no measurable inhibition of IL-6 classical signalling, or more preferably cause (or elicit) no significant inhibition (preferably no statistically significant inhibition) of IL-6 classical signalling.

In some embodiments, antibodies (or antigen binding proteins) that maintain IL-6 classical signalling can be determined by performing an appropriate test (or assay) to determine (or quantify) the level of (or amount of or residual) IL-6 classical signalling through the membrane-bound IL-6R. Suitable assays for measuring IL-6 classical signalling are described elsewhere herein and can be used. For example, in preferred embodiments, levels or values for IL-6 classical signalling (or the inhibition of IL-6 classical signalling) are determined using a cell proliferation or intracellular IL-6 signalling assay, e.g. as described herein. However, any assay measuring IL-6 classical signalling is suitable for (and can be used) to determine the maintenance of IL-6 classical signalling as defined herein, although cell-based assays are preferred.

In addition, an appropriate concentration of antibody (binding protein) is used in such assays. Thus, in some embodiments, antibodies (or binding proteins) that maintain IL-6 classical signalling are determined when the antibody (or binding protein) is used at a concentration of 1 or 10 μg/ml. Thus, in preferred embodiments the above levels or values of IL-6 classical signalling apply when the antibodies (or binding proteins) are used at a concentration of 1 or 10 μg/ml, for example 10 μg/ml.

In a preferred embodiment, the antibodies (or antigen binding proteins) of the present invention have the ability to inhibit IL-6 trans signalling at a concentration of 10 μg/ml (or 53 nM), and maintain IL-6 classical signalling at a concentration of 10 μg/ml (or 53 nM), preferably when an anti-IL-6R IgG is used.

In a preferred embodiment, the antibodies (or antigen binding proteins) of the present invention have the ability to inhibit IL-6 trans signalling at a concentration of 3.3 μg/ml (or 53 nM), and maintain IL-6 classical signalling at a concentration of 3.3 μg/ml (or 53 nM), preferably when an anti-IL-6R Fab fragment is used.

Alternatively, the maintenance of IL-6 classical signalling (or equivalent terms) as referred to herein, by an antibody (or antigen binding protein) of the invention, can be determined by calculating the IC50 value (or minimum IC50 value) (i.e. the concentration of antibody (or antigen binding protein) required to inhibit a biological process, for example cell proliferation, by a half (50%) of IL-6 classical signalling using the % inhibitions of IL-6 classical signalling over an increasing concentration range of antibody (e.g. 0.01, 0.1, 0.3, 0.5, 0.7, 1, 5 and 10 μg/ml of anti-IL-6R IgG antibody, or 3.3, 33.3, 100, 166.7, 233.3, 333.3 or 3300 ng/ml anti-IL-6R Fab) as defined elsewhere herein. The skilled person will be familiar with calculating IC50 values based on the % inhibitions of IL-6 classical signalling as defined elsewhere herein. If the antibody (or antigen binding protein) does not inhibit the biological process, for example cell proliferation, by a half (50%)) at any concentration, then a minimum value for the IC50 can be calculated, e.g. if the antibody (or antigen binding protein) does not inhibit cell proliferation by a half (50%) at the maximum tested concentration 10 μg/ml, than the IC50 value is greater than 10 μg/ml (>10 μg/ml).

In some embodiments, the maintenance of IL-6 classical signalling by an antibody (or antigen binding protein) of the invention is defined by an IC50 of IL-6 classical signalling of at least 100 nM, at least 95 nM, at least 90 nM, at least 85 nM, at least 80 nM, at least 75 nM, at least 70 nM, at least 65 nM, at least 60 nM, at least 55 nM, at least 50 nM, at least 45 nM, at least 40 nM, at least 35 nM, at least 30 nM, at least 25 nM, or at least 20 nM, preferably when the IC50 is calculated using a cell-based assay, preferably a cell-based proliferation assay, e.g. as described herein.

In preferred embodiments, the maintenance of IL-6 classical signalling by an antibody (or antigen binding protein) of the invention is defined by an IC50 of IL-6 classical signalling of at least 20 nM, or at least 25 nM, or at least 30 nM, or at least 35 nM, or at least 40 nM, or at least 45 nM, or at least 50 nM, or at least 53 nM, preferably when the IC50 is calculated using a cell-based assay, preferably a cell-based proliferation assay, e.g. as described herein.

Alternatively, in some embodiments, the maintenance of IL-6 classical signalling by an antibody (or antigen binding protein) of the invention is defined by an IC50 of IL-6 classical signalling of at least 5 μg/ml, at least 10 μg/ml, at least 15 μg/ml, at least at least 20 μg/ml, at least 25 μg/ml, at least 30 μg/ml, at least 35 μg/ml, at least 40 μg/ml, at least 45 μg/ml, or at least 50 μg/ml, preferably when an anti-IL-6R IgG antibody is used, preferably when the IC50 is calculated using a cell-based assay, preferably a cell-based proliferation assay, e.g. as described herein.

In preferred embodiments, the maintenance of IL-6 classical signalling by an antibody (or antigen binding protein) of the invention is defined by an IC50 of IL-6 classical signalling of at least 5 μg/ml, at least 6 μg/ml, at least 7 μg/ml, at least 8 μg/ml, at least 9 μg/ml, or at least 10 μg/ml, preferably when an anti-IL-6R IgG antibody is used, preferably when the IC50 is calculated using a cell-based assay, preferably a cell-based proliferation assay, e.g. as described herein.

In some embodiments, the above levels or values for IL-6 classical signalling apply when the antibody is a monoclonal antibody (e.g. as opposed to a polyclonal antibody). In preferred embodiments, the above levels or values apply when the antibody is a monoclonal full length (whole) antibody, e.g. a chimeric full length (whole) antibody, or a Fab fragment, most preferably a monoclonal full length (whole) antibody, e.g. a chimeric full length (whole) antibody.

For example, the exemplified antibodies of the invention have been shown to inhibit IL-6 classical signalling by no more than 12%, 9%, 8%, 7%, or 6%, when assessed as chimeric full length (whole) antibodies using a cell proliferation assay at a concentration of 10 μg/ml.

For example, the exemplified anti-IL-6R IgG antibodies of the invention have an IC50 of IL-6 classical signalling of at least 53 nM (or 10 μg/ml), when using a cell-based proliferation assay, e.g. as described herein.

Thus, in some embodiments, the anti-IL-6R IgG antibodies (or antigen binding domains) of the present invention maintain IL-6 classical signalling, preferably wherein the maintenance of IL-6 classical signalling is defined by an IC50 of at least 53 nM (or 10 μg/ml) when using a cell-based cell proliferation assay, e.g. as described herein.

The exemplified anti-IL-6R IgG antibodies of the invention do not inhibit (or significantly inhibit) (i.e. maintain) IL-6 classical signalling at the highest tested concentration of 10 μg/ml (or 53 nM).

Thus, in some embodiments, the anti-IL-6R IgG antibodies (or antigen binding proteins) of the present invention have the ability to maintain IL-6 classical signalling, as defined elsewhere herein, at a concentration of 1 μg/ml or 10 μg/ml, preferably at a concentration of 10 μg/ml (or 53 nM). As indicated above, in this aspect of the invention antibodies (or antigen binding proteins) inhibit IL-6 trans signalling through the soluble IL-6R while maintaining IL-6 classical signalling through the membrane-bound IL-6R. This means that the given antibody (or binding protein) inhibits (or is capable of inhibiting) IL-6 trans signalling to a greater extent than it inhibits (or is capable of inhibiting) IL-6 classical signalling. Thus, if a given antibody (or binding protein) inhibits IL-6 trans signalling by X %, the same antibody (or binding protein) will inhibit IL-6 classical signalling by <X %. This property can also be referred to herein as preferential or selective inhibition.

In some embodiments, the % inhibition (or % inhibition value) of IL-6 trans signalling is at least 5% higher, at least 10% higher, at least 15% higher, at least 20% higher, at least 25% higher, at least 30% higher, at least 35% higher, at least 40% higher, at least 45% higher, at least 50% higher, at least 55% higher, at least 60% higher, at least 65% higher, at least 70% higher, at least 75% higher, at least 80% higher, at least 85% higher, at least 90% higher, at least 95% higher, or 100% higher than the % inhibition (or % inhibition value) of IL-6 classical signalling.

In preferred embodiments, to compare the % inhibition (or % inhibition value) of IL-6 trans signalling as compared to (or relative to) IL-6 classical signalling, typically % inhibitions (% inhibition values) from the same assay (e.g. measuring cell proliferation or measuring intracellular IL-6 signalling) are used, and wherein the same antibody (or binding protein) concentration is used in said assay (e.g. 1 μg/ml or 10 μg/ml). In such embodiments, the % inhibition (or % inhibition value) of IL-6 trans signalling is at least 15% higher, but typically at least 50% higher, at least 60% higher, preferably at least 75% higher, at least 80% higher, at least 85% higher, at least 90% higher, at least 95% higher or 100% higher than the % inhibition (or % inhibition value) of IL-6 classical signalling.

For example, the exemplified antibodies of the invention have been shown to have an ability to inhibit IL-6 trans signalling relative to IL-6 classical signalling of at least 68%, 80%, 81% or 82% when assessed as chimeric full length (whole) antibodies using a cell proliferation assay at a concentration of 10 μg/ml.

In some embodiments, % inhibition (or % inhibition value) of IL-6 signalling is determined by performing a test (or assay) to determine (or quantify) the level of (or amount of) inhibition of IL-6 trans signalling through the soluble IL-6R and/or a test (or assay) to determine (or quantify) the level of (or amount of) inhibition of IL-6 classical signalling through the membrane-bound IL-6R. Suitable assays are described elsewhere herein, for example assays that measure IL-6 classical signalling, IL-6 trans signalling and IL-6 mixed (combined) signalling are described herein and can be used, for example in some embodiments assays that measure IL-6 classical signalling and IL-6 trans signalling as described herein can be used. However, any assays measuring IL-6 trans signalling and IL-6 classical signalling are suitable for (and can be used) to determine the % inhibition (or % inhibition value) of IL-6 trans signalling relative to IL-6 classical signalling as defined herein.

In preferred embodiments, to determine the % inhibition (or % inhibition value) of IL-6 trans signalling relative to IL-6 classical signalling, values for IL-6 classical signalling and IL-6 trans signalling are determined using the same assay (e.g. a cell proliferation or intracellular IL-6 signalling, e.g. as described herein).

In addition, typically, % inhibition (or % inhibition value) of IL-6 trans signalling and % inhibition (or % inhibition value) of IL-6 classical signalling is determined when the antibody (or binding protein) is used at the same concentration in both such assays (i.e. the same concentration of antibody is used in the test to determine the % inhibition of IL-6 trans signalling and the % inhibition of IL-6 classical signalling).

In some embodiments, the above % inhibitions (or % inhibition values) are determined when the antibody (or binding protein) is used at a concentration of 0.01, 0.1, 0.3, 0.5, 0.7, 1 or 10 μg/ml, preferably 1-10 μg/ml. Thus, in preferred embodiments the above % inhibitions (or % inhibition values) apply when the antibodies (or binding proteins) are used at a concentration of 1 or 10 μg/ml, for example 10 μg/ml.

In some embodiments, the above % inhibitions (or % inhibition values) apply when the antibody is a monoclonal antibody (e.g. as opposed to a polyclonal antibody). In preferred embodiments, the above % inhibitions (or % inhibition values) apply when the antibody is a monoclonal full length (whole) antibody, e.g. a chimeric full length (whole) antibody, or a Fab fragment, most preferably a monoclonal full length (whole) antibody, e.g. a chimeric full length (whole) antibody.

Alternatively viewed, the present invention provides an antigen binding protein, for example an antibody, for example an isolated antibody (or antigen binding protein), comprising at least one, preferably two, antigen binding domains which binds to human IL-6R, wherein said antigen binding protein or antibody preferentially inhibits, or selectively inhibits, IL-6 trans signalling through the human soluble IL-6R, e.g. as opposed to (or when compared with) IL-6 classical signalling through the human membrane-bound IL-6R. Discussion of various features of the antibodies (or antigen binding proteins) of other aspects of the invention and preferred embodiments apply mutatis mutandis to this aspect of the invention.

Nucleic acid molecules (e.g. one or more nucleic acid molecules) comprising nucleotide sequences that encode the antigen binding proteins (e.g. antibodies) of the present invention as defined herein, or parts or fragments thereof, or nucleic acid molecules substantially homologous thereto (for example having at least 80% sequence identity to specific nucleic acid sequences defined herein), form yet further aspects of the invention.

Preferred nucleic acid molecules are those encoding an antibody (or antigen binding protein) of the present invention as described elsewhere herein which can bind to human IL-6R, e.g. an antibody (or antigen binding protein) of the invention with CDR and optionally FR and other regions as defined in any one of Tables A or B or C or D or E or F, or antibodies with sequences substantially homologous thereto.

Preferred nucleic acid molecules are those encoding an antibody of the present invention which can bind to human IL-6R (e.g. comprising nucleic acid sequences encoding SEQ ID NO:3 and/or SEQ ID NO:4, SEQ ID NO:21 and/or SEQ ID NO:22, SEQ ID NO:39 and/or SEQ ID NO:40, SEQ ID NO:57 and/or SEQ ID NO:58, or SEQ ID NO:75 and/or SEQ ID NO:76, such as SEQ ID NO:1 and/or SEQ ID NO:2, SEQ ID NO:19 and/or SEQ ID NO:20, SEQ ID NO:37 and/or SEQ ID NO:38, SEQ ID NO:55 and/or SEQ ID NO:56, or SEQ ID NO:73 and/or SEQ ID NO:74, respectively).

Preferred nucleic acid molecules are those encoding a VH region of an antibody (or antigen binding protein) of the present invention (e.g. those encoding SEQ ID NOs:3 or 21 or 39 or 57 or 75, such as SEQ ID NOs:1 or 19 or 37 or 55 or 73, respectively). Other preferred nucleic acid molecules are those encoding a VL region of an antibody (or antigen binding protein) of the present invention (e.g. those encoding SEQ ID NOs:4 or 22 or 40 or 58 or 76, such as SEQ ID NOs:2 or 20 or 38 or 56 or 74, respectively). Other preferred nucleic acid molecules are those encoding a VH region of an antigen binding protein (e.g. antibody) of the present invention and a VL region of an antigen binding protein (e.g. antibody) of the present invention.

Thus, preferred nucleic acid molecules comprise sequences which encode a heavy chain variable region (VH) that has the amino acid sequence of SEQ ID NOs:3 or 21 or 39 or 57 or 75 (which is preferably encoded by SEQ ID NOs:1 or 19 or 37 or 55 or 73, respectively) and/or comprise sequences which encode a light chain variable region (VL) which has the amino acid sequence of SEQ ID NOs:4 or 22 or 40 or 58 or 76 (which is preferably encoded by SEQ ID NOs:2 or 20 or 38 or 56 or 74, respectively).

Also preferred are nucleic acids which encode the following combinations: SEQ ID NOs:3 and 4; or SEQ ID NOs:21 and 22; or SEQ ID NOs:39 and 40; or SEQ ID NOs:57 and 58; or SEQ ID NOs:75 and 76.

Also preferred are nucleic acid molecules which comprise the following combinations: SEQ ID NOs:1 and 2; or SEQ ID NOs:19 and 20; or SEQ ID NOs:37 and 38; or SEQ ID NOs:55 and 56; or SEQ ID NOs:73 and 74.

Other preferred nucleic acid molecules comprise sequences that encode human IgG (preferably IgG1) forms of the antibodies of the invention, e.g. sequences encoding the VH and VL regions of the antibodies of the invention (e.g. as described in Tables A, B, C, D and E) chimerized to (or connected to) human IgG (preferably IgG1).

Other preferred nucleic acid molecules comprise sequences that encode a Fab format or other fragment of the antibodies of the invention (preferably a Fab format of the antibody C20, or a Fab format of the antibody C07, C12, C19 or C09).

In another aspect, the present invention provides a set (or plurality) of nucleic acid molecules each comprising a nucleotide sequence (e.g. comprising a nucleotide sequence encoding a heavy chain variable region or comprising a nucleotide sequence encoding a light chain variable region), wherein said set of nucleic acid molecules together (or collectively) encode an antibody (or antigen binding protein) in accordance with the invention. Such a set of nucleic acid molecules may be characterised in that when the set is expressed (i.e. expressed together), e.g. in a host cell, an entire antibody (or binding protein) of the present invention is expressed and preferably assembled.

In some embodiments, the set of nucleic acid molecules comprises at least one nucleic acid molecule comprising a nucleotide sequence encoding a heavy chain variable region, and at least one nucleic acid molecule comprising a nucleotide sequence encoding a light chain variable region as described herein, preferably wherein the nucleic acid molecule comprising a nucleotide sequence encoding a heavy chain variable region and the nucleic acid molecule comprising a nucleotide sequence encoding a light chain variable region are a preferred combination of nucleic acid molecules as described above.

Nucleic acid sequences that are substantially homologous to the sequences described above may also be used.

The term “nucleic acid sequence” or “nucleic acid molecule” as used herein refers to a sequence of nucleoside or nucleotide monomers composed of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid molecules may be double stranded or single stranded. The nucleic acid molecules may be wholly or partially synthetic or recombinant.

Antigen binding proteins (e.g. antibodies) of the invention comprising one or more (e.g. one, two, three, four, five or six) CDR sequences that are “substantially homologous” to one or more of the CDRs of the exemplified antibodies of the invention, and antigen binding proteins (e.g. antibodies) of the invention comprising heavy and/or light chain variable domains that comprise an amino acid sequence that is substantially homologous to the VH and/or VL sequences of the exemplified antibodies of the invention, are termed “substantially homologous antigen binding proteins” (e.g. “substantially homologous antibodies”) herein. Thus, “substantially homologous antigen binding proteins” (e.g. “substantially homologous antibodies”) are any such antigen binding protein/antibody comprising one or more CDR or variable domain sequence that is substantially homologous to the sequence of a specific CDR or variable domain sequence described herein, e.g. with a given SEQ ID NO herein. The substantially homologous antigen binding proteins (e.g. antibodies) as referred to herein, are expressly limited to those that retain one or more of the properties of the antigen binding proteins (e.g. antibodies) described herein, e.g. with given SEQ ID NOs specified herein, in particular the ability to bind human IL-6R and preferably the ability to inhibit IL-6 trans signalling through the human soluble IL-6 receptor while maintaining (e.g. not significantly affecting or not significantly reducing) IL-6 classical signalling through the human membrane-bound IL-6 receptor, and preferably the ability to bind to human soluble IL-6R and human membrane-bound IL-6R.

The term “substantially homologous” as used herein in connection with an amino acid or nucleic acid sequence includes sequences having at least 55%, 60%, 65%, 70% or 75%, preferably at least 80%, and even more preferably at least 85%, 90%, 95%, 96%, 97%, 98% or 99%, sequence identity to the amino acid or nucleic acid sequence disclosed. Substantially homologous sequences of the invention thus include single or multiple base or amino acid alterations (additions, substitutions, insertions, or deletions) to the sequences of the invention.

At the amino acid level preferred substantially homologous sequences contain up to 6, e.g. only 1, 2, 3, 4, 5 or 6, for example 1, 2, 3, 4 or 5, preferably 1, 2, 3 or 4, preferably 1, 2 or 3, more preferably 1 or 2 (more preferably 1), altered amino acids, in one or more of the framework regions and/or one or more of the CDRs making up the sequences of the invention.

Other preferred examples of “substantially homologous” sequences are sequences having at least 50%, at least 55%, at least 60%, at least 65%, at least 70%, at least 75%, at least 80%, at least 85%, at least 90%, or at least 95%, amino acid sequence identity to the amino acid sequence of one or more of the CDR regions or one or more of the FR regions disclosed in Tables A, B, C, D or E. Thus, in some embodiments, a “substantially homologous” CDR sequence may be a sequence having at least 50%, at least 55%, at least 60%, at least 65%, at least 70%, at least 75%, at least 80%, at least 85%, at least 90% or at least 95% sequence identity to a given CDR sequence described herein.

In some embodiments, in antibodies (or antigen binding proteins) having a “substantially homologous” sequence as compared to a given sequence, or having a certain degree of sequence identity as compared to a given sequence, the altered amino acid residues(s) are not in a CDR region. For example, in some embodiments, in antibodies (or antigen binding proteins) having a VH domain that has a certain degree of sequence identity to a given VH domain sequence of a particular antibody (or binding protein) of the invention, the altered (or variant) residue(s) are not in a CDR region. Thus, in some embodiments, in antibodies (or antigen binding proteins) having a “substantially homologous” sequence as compared to a given sequence, or having a certain degree of sequence identity as compared to a given sequence, the altered amino acid residues(s) are in one or more framework regions.

As is evident from elsewhere herein, in other embodiments, in antibodies (or antigen binding proteins) having a “substantially homologous” sequence as compared to a given sequence, or having a certain degree of sequence identity as compared to a given sequence, the altered amino acid residues(s) may be in a CDR region.

In some embodiments, in an antibody (or antigen binding protein) having a “substantially homologous” sequence as compared to a given sequence, or having a certain degree of sequence identity as compared to a given sequence, the three VH CDR amino acid sequences (i.e. all three VH CDR sequences taken together) and the three VL CDR amino acid sequences (i.e. all three VL CDR sequences taken together), to make up a set of six CDRs in total, are considered together to be the whole (or entire) CDR complement of the antibody (or antigen binding protein), and the amino acid sequence of said whole CDR complement of said antibody (or antigen binding protein) is at least 70%, preferably at least 80%, or at least 85%, or at least 90%, or at least 95% identical to the corresponding whole (or entire) CDR complement of a given starting (or reference) antibody (or antigen binding protein). The starting (or reference) antibody (or antigen binding protein) may have the CDR sequences of the C07, C09, C12, C19 or C20 antibodies of the present invention as shown in Tables A, B, C, D and E, respectively.

In addition, at the amino acid level, preferred substantially homologous sequences contain up to 6, e.g. only 1, 2, 3, 4, 5 or 6, for example 1, 2, 3, 4 or 5, preferably 1, 2, 3 or 4, preferably 1, 2 or 3, more preferably 1 or 2 (preferably 1), altered amino acids, in the combined framework regions (e.g. the four framework regions), and/or the combined CDRs (e.g. the three CDR regions) making up the VL or VH domains of the antibodies (or antigen binding proteins) of the invention. In addition, at the amino acid level, preferred substantially homologous sequences contain up to 6, e.g. only 1, 2, 3, 4, 5 or 6, for example 1, 2, 3, 4 or 5, preferably 1, 2, 3 or 4, preferably 1, 2 or 3, more preferably 1 or 2 (preferably 1), altered amino acids, in the VH domain and/or the VL domain of the antibodies (or antigen binding proteins) of the invention.

In certain embodiments, if a given starting sequence is relatively short (e.g. three or eight amino acids in length), then fewer amino acid substitutions may be present in sequences substantially homologous thereto as compared with the number of amino acid substitutions that might optionally be made in a sequence substantially homologous to a longer starting sequence. For example, in certain embodiments, a sequence substantially homologous to a starting VH CDR1 sequence in accordance with the present invention, e.g. a starting VH CDR1 sequence which is eight amino acid residues in length, preferably has 1 or 2 or 3 (more preferably 1 or 2, e.g. 1) altered amino acids in comparison with the starting sequence. A sequence substantially homologous to a starting VL CDR2 sequence in accordance with the present invention, e.g. a starting VL CDR2 sequence which is three amino acid residues in length, preferably has 1 or 2, more preferably has 1, altered amino acid in comparison with the starting sequence. Accordingly, in some embodiments the number of altered amino acids in substantially homologous sequences (e.g. in substantially homologous CDR sequences) can be tailored to the length of a given starting CDR sequence. For example, different numbers of altered amino acids can be present depending on the length of a given starting CDR sequence such as to achieve a particular % sequence identity in the CDRs, for example a sequence identity of at least 55%, 60%, 65%, 70%, 75%, 80%, 85%, 90%, 95%, 96%, 97%, 98% or 99%.

In all embodiments, altered residues might be conserved or non-conserved amino acid substitutions, or a mixture thereof.

In such embodiments, preferred alterations are conservative amino acid substitutions.

Routine methods in the art such as alanine scanning mutagenesis and/or deep mutational scanning (which aims to perform all possible mono-substitutions on all selected residues within a given protein sequence) and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

Once identified, the addition, deletion, substitution or insertion of one or more amino acids in the amino acid sequence of a parent antibody (or antigen binding protein) to form a new antibody (or antigen binding protein), wherein said parent antibody (or binding protein) is one of the antibodies (or antigen binding proteins) of the invention as defined elsewhere herein, and testing the resulting new antibody (or antigen binding protein) to identify antibodies (or antigen binding proteins) that bind to human IL-6R in accordance with the invention can be carried out using techniques which are routine in the art and examples are described elsewhere herein. Such methods can be used to form multiple new antibodies (or antigen binding proteins) that can all be tested for their ability to bind human IL-6R. Preferably said addition, deletion, substitution or insertion of one or more amino acids takes place in one or more of the CDR domains.

For example, said manipulations could conveniently be carried out by genetic engineering at the nucleic acid level wherein nucleic acid molecules encoding appropriate antibodies (or antigen binding proteins) and domains thereof are modified such that the amino acid sequence of the resulting expressed protein is in turn modified in the appropriate way. Testing the ability of one or more of the modified antibodies (or antigen binding proteins) to bind to human IL-6R can be carried out by any appropriate method, which are well known and described in the art. Suitable methods are also described elsewhere herein and in the Examples section.

New antibodies (or antigen binding proteins) produced, obtained or obtainable by these methods form a yet further aspect of the invention.

The term “substantially homologous” also includes modifications or chemical equivalents of the amino acid and nucleotide sequences of the antibodies (or binding proteins) of the present invention that perform substantially the same function as the proteins or nucleic acid molecules of the antibodies (or binding proteins) of the invention in substantially the same way. For example, any substantially homologous antibody (or binding protein) should retain the ability to bind to human IL-6R as described above. Preferably, any substantially homologous antibody (or binding protein) should retain one or more (or all) of the functional capabilities of the starting antibody (or binding protein), including the capability of inhibiting IL-6 trans signalling while maintaining (e.g. not inhibiting or not significantly inhibiting) IL-6 classical signalling, and preferably the ability to bind to human soluble IL-6R and human membrane-bound IL-6R.

Substantially homologous sequences of proteins of the invention include, without limitation, conservative amino acid substitutions, or for example alterations that do not affect the VH, VL or CDR domains of the antibodies (or antigen binding proteins), e.g. antibodies (or antigen binding proteins) where tag sequences, labels, or other components are added that do not contribute to the binding of IL-6R antigen, or alterations to convert one type or format of antibody molecule or fragment to another type or format of antibody molecule or fragment (e.g. conversion from Fab to scFv or whole antibody or vice versa), or the conversion of an antibody molecule to a particular class or subclass of antibody molecule (e.g. the conversion of an antibody molecule to IgG or a subclass thereof, e.g. IgG₁).

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine). In other examples, families of amino acid residues can be grouped based on hydrophobic side groups or hydrophilic side groups.

Homology or sequence identity may be assessed by any convenient method. However, for determining the degree of homology or identity between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally recognized in the art and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention 25 having at least 55%, 60%, 65%, 70%, 75%, 80%, 85%, 90%, 95%, 96%, 97%, 98% or 99% homology, sequence identity etc. may be determined using the ALIGN program with default parameters (for instance available on Internet at the GENESTREAM network server, IGH, Montpellier, France).

In all embodiments, antibodies (or antigen binding proteins) of the invention containing substantially homologous sequences retain the ability to bind to human IL-6R. Preferably, antibodies (or antigen binding proteins) containing substantially homologous sequences retain one or more (preferably all) of the other properties described herein in relation to the antibodies of the invention, e.g. the C07 and/or C09 and/or C12 and/or C19 and/or C20 antibody. Thus, preferred antibodies (or antigen binding proteins) containing substantially homologous sequences retain the property of binding to human IL-6R and inhibiting IL-6 trans signalling through the human soluble IL-6R while maintaining (e.g. not significantly affecting or not significantly reducing) IL-6 classical signalling through the human membrane-bound IL-6R.

In some embodiments, antibodies (or antigen binding proteins) of the invention containing substantially homologous sequences bind to (or specifically bind to) human IL-6R and inhibit IL-6 trans signalling while maintaining IL-6 classical signalling, and further possess one or more, e.g. two or three, of the following properties:
i. bind to the soluble and/or (preferably “and”) the membrane-bound human IL-6R;
ii. do not bind to IL-6R (soluble and/or (preferably “and”) membrane-bound IL-6R) in some non-human species, e.g. murine (or mouse), and/or (preferably “and”) maintain (or do not inhibit or do not significantly inhibit), or do not affect IL-6 classical signalling or IL-6 trans signalling in some non-human species, e.g. through the appropriate non-human IL-6R, e.g. murine (or mouse) IL-6R;
iii. maintain (or do not inhibit or do not significantly inhibit) other gp130-dependent signalling pathways, e.g. maintain (or do not inhibit or do not significantly inhibit) IL-11 signalling.

In some embodiments, the antibodies (or binding proteins) of the present invention containing substantially homologous sequences further possess one or both of the following properties:
iv. do not preferentially bind (or do not bind with a greater affinity or significantly greater affinity) to human IL-6/IL-6R complex (soluble and/or membrane human IL-6/IL-6R complex) as compared to human IL-6R (soluble and/or membrane-bound human IL-6R) alone;
v. do not affect (or do not significantly affect) the IL-6 buffering system (or do not reduce the level of or amount of (or do not significantly reduce the level of or amount of) free IL-6 in the extracellular space).

Methods used to determine binding of substantially homologous antibody (or antigen binding protein) sequences to human IL-6R, e.g. human soluble and/or membrane bound IL-6R are well known in the art and described elsewhere herein.

Methods used to determine whether substantially homologous antibody (or antigen binding protein) sequences inhibit IL-6 trans signalling while maintaining IL-6 classical signalling are described elsewhere herein. Thus, substantially homologous antibody (or binding protein) sequences would be expected to induce a similar (or not significantly different) classical:trans signalling ratio and/or a similar (or not significantly different) % inhibition (or % inhibition value) of IL-6 trans signalling and/or IL-6 classical signalling to those as described elsewhere herein for the antibodies of the invention, e.g. for the C07 and/or C09 and/or C12 and/or C19 and/or C20 antibodies.

Further examples of substantially homologous amino acid sequences in accordance with the present invention are described elsewhere herein.

Preferably, any substantially homologous antibody (or antigen binding protein) of the invention should retain the ability to bind to, or specifically bind to, the same epitope of human IL-6R as recognized by the starting antibody (or binding protein) in question, for example, the same epitope recognized by the CDR domains of one or more of the antibodies (or binding proteins) of the invention or the VH and VL domains of one or more of the antibodies (or binding proteins) of the invention as described herein, e.g. bind to the same epitope as one or more of the various antibodies of the invention (e.g. one or more of the anti-IL-6R antibodies as shown in Tables A, B, C, D or E).

Binding to the same epitope can be readily tested using the known protein sequence (or multiple known protein sequences) of the antibodies of present invention (e.g. the VH and/or VL domain sequences of C07 and/or C09 and/or C12 and/or C19 and/or C20 as disclosed in the various sequence Tables herein), using methods well known and described in the art, e.g. using binding assays, e.g. a competition assay. Suitable binding assays are discussed elsewhere herein. Thus, preferably, any substantially homologous antigen binding protein (e.g. antibody) should retain the ability to compete with one or more of the antigen binding proteins of the invention for binding to the relevant antigen. Binding to the same epitope can, for example also be tested using epitope mapping assays, e.g. by analysis of the crystal structure of the antigen-antibody complex, or by mutational studies of individual residues (e.g. using alanine scanning and/or deep mutational scanning, DMS, for example yeast display in combination with DMS, see for example as described in Sierocki et al., 2021, PLoS Negl Trop Dis., 15(3):e0009231; see also Van Blarcom et al., 2015, JMB, 427:6(B):1513-1534 and Medina-Cucurella and Whitehead, 2018, Methods Mol. Biol., 1764:101-121). Any of the above such analysis to determine the epitope can be used.

Thus, antibodies (or binding proteins) which bind to the same epitope as one or more of the various antibodies of the invention, for example as assessed or determined by one or more of the methods outlined above, form yet further aspects of the invention.

Thus, a person skilled in the art will appreciate that such methods can be used to test whether any antibodies or binding proteins, for example “substantially homologous” antibodies, have the same binding specificities, e.g. bind to the same epitope, as the antibodies of the invention. The skilled person will be aware of other suitable methods and variations.

As outlined below, a competition binding assay can be used to test whether antibodies (or binding proteins), for example “substantially homologous” antibodies (or binding proteins) retain the ability to bind to, or specifically bind to, the same (or substantially the same) epitope of human IL-6R as recognized by one or more of the antibodies of the invention as shown in the various sequence Tables herein, or have the ability to compete with one or more of the various antibodies of the invention as shown in the various sequence Tables herein. The method described below is only one example of a suitable competition assay. The skilled person will be aware of other suitable methods and variations.

An exemplary competition assay involves assessing the binding of various effective concentrations of an antibody (or antigen binding protein) of the invention to human IL-6R in the presence of varying concentrations of a test antibody (or binding protein) (e.g. a substantially homologous antibody). The amount of inhibition of binding induced by the test antibody/binding protein can then be assessed. A test antibody/binding protein that shows increased competition with an antibody of the invention at increasing concentrations (i.e. increasing concentrations of the test antibody/binding protein result in a corresponding reduction in the amount of antibody of the invention binding to IL-6R) is evidence of binding to the same or substantially the same epitope. Preferably, the test antibody/binding protein significantly reduces the amount of antibody of the invention that binds to IL-6R. Preferably, the test antibody/binding protein reduces the amount of antibody of the invention that binds to IL-6R by at least about 95%. ELISA or flow cytometry assays may be used for assessing inhibition of binding in such a competition assay but other suitable techniques would be well known to a person skilled in the art.

Thus, a yet further aspect of the invention provides an antibody (or binding protein), which binds to (or specifically binds to) human IL-6R and which has the ability to bind to the same (or substantially the same) epitope as the C07 (Table A) and/or C09 (Table B) and/or C12 (Table C) and/or C19 (Table D) and/or C20 (Table E) antibody, i.e. the ability to bind to the same (or substantially the same) epitope as an antibody (or binding protein) comprising the same CDRs, or the same VH and VL domains, as the C07 (Table A) and/or C09 (Table B) and/or C12 (Table C) and/or C19 (Table D) and/or C20 (Table E) antibody. In a yet further aspect, antibodies (or binding proteins) which have the ability to compete with one or more of the various antibodies of the invention (e.g. to compete with one or more of the antibodies as shown in Tables A, B, C, D or E as set out above) for binding to IL-6R are further embodiments of the invention. Other features and properties of other aspects of the invention apply, mutatis mutandis, to this aspect of the invention.

The term “competing antibodies (or antigen binding proteins)”, as used herein, refers to antibodies (or antigen binding proteins) that bind to about, substantially or essentially the same, or even the same, epitope as a “reference antibody”. Competing antibodies are thus able to effectively compete with a reference antibody for binding to human IL-6R. Preferably, the competing antibody can bind to the same epitope as the reference antibody. Alternatively viewed, the competing antibody preferably has the same epitope specificity as the reference antibody.

“Reference antibodies” as used herein are antibodies which can bind to human IL-6R in accordance with the invention which preferably have a VH and a VL domain as defined herein in Tables A, B, C, D or E.

The identification of one or more antibodies (or binding proteins) that bind to the same epitope, or competing antibodies (or binding proteins), is a straightforward technical matter now that reference antibodies such as those outlined in the sequence Tables herein have been provided. As the identification of competing antigen binding proteins (e.g. antibodies) is determined in comparison to a reference antigen binding protein (e.g. antibody), it will be understood that actually determining the epitope to which either or both antigen binding proteins bind is not in any way required in order to identify a competing antigen binding protein. However, epitope mapping can be performed using standard techniques, if desired. Such epitope mapping can also be supplemented with competition assays, e.g. as an initial or supplementary screening step, which can be performed using standard techniques. In this regard for example, anti-IL-6R antibodies can be generated by immunization protocols using human soluble and/or membrane-bound IL-6R antigen, e.g. as described in the Examples section, after which such anti-IL-6R antibodies can readily be screened, for example using methods as described herein, to identify those that bind to the same epitope as the reference antibodies of the invention, and preferably therefore show the same functional properties, e.g. the ability to inhibit IL-6 trans signalling while maintaining IL-6 classical signaling, as described herein. Alternatively, substantially homologous sequences derived from antibodies with the sequences shown in Tables A, B, C, D or E can be screened in this way.

The terms “antibody”, and “immunoglobulin”, as used herein, refer broadly to any immunological binding agent that comprises an antigen binding domain, including polyclonal and monoclonal antibodies. Monoclonal antibodies are preferred.

The term “antibody”, as used herein, refers to both full length (whole) antibodies and antibody fragments (e.g. Fab fragments), which form an additional aspect of the invention.

However, antibodies and antigen binding proteins of the invention have a structure or format such that they bind to human IL-6R, e.g. can comprise antibodies or antibody fragments, or antigen binding domains (e.g. of antibodies or antibody fragments), that bind to human IL-6R.

Depending on the type of constant domain in the heavy chains, whole antibodies are assigned to one of five major classes: IgA, IgD, IgE, IgG, and IgM and the antibodies of the invention may be in any one of these classes. Several of these are further divided into subclasses or isotypes, such as IgG1, IgG2, IgG3, IgG4, and the like. The heavy-chain constant domains that correspond to the different classes of immunoglobulins are termed α, δ, ε, γ and μ, respectively. The subunit structures and three-dimensional configurations of different classes of immunoglobulins are well known.

Generally, where whole antibodies are used in the invention, IgG, and in particular IgG1, are preferred.

The “light chains” of mammalian antibodies are assigned to one of two clearly distinct types: kappa (κ) and lambda (λ), based on the amino acid sequences of their constant domains and some amino acids in the framework regions of their variable domains. For antibodies of the invention, kappa (κ) light chains are sometimes preferred.

The term “heavy chain complementarity determining region” (“heavy chain CDR”) as used herein refers to regions of hypervariability within the heavy chain variable region (VH domain) of an antibody molecule. The heavy chain variable region has three CDRs termed heavy chain CDR1, heavy chain CDR2 and heavy chain CDR3 from the amino terminus to carboxy terminus. The heavy chain variable region also has four framework regions (FR1, FR2, FR3 and FR4 from the amino terminus to carboxy terminus). These framework regions separate the CDRs.

The term “heavy chain variable region” (VH domain) as used herein refers to the variable region of a heavy chain of an antibody molecule.

The term “light chain complementarity determining region” (“light chain CDR”) as used herein refers to regions of hypervariability within the light chain variable region (VL domain) of an antibody molecule. Light chain variable regions have three CDRs termed light chain CDR1, light chain CDR2 and light chain CDR3 from the amino terminus to the carboxy terminus. The light chain variable region also has four framework regions (FR1, FR2, FR3 and FR4 from the amino terminus to carboxy terminus). These framework regions separate the CDRs.

The term “light chain variable region” (VL domain) as used herein refers to the variable region of a light chain of an antibody molecule.

As described elsewhere herein, antibodies, antibody fragments (e.g. Fab fragments) and antigen binding proteins of the invention have a structure or format such that they are capable of binding to human IL-6R. Any appropriate format may be used, e.g. any format of antibody or antibody fragment which contains at least one antigen binding domain that can bind to human IL-6R. Exemplary and preferred formats or fragments are full length (whole) antibodies such as IgG1, IgG2, IgG3, IgG4, IgA1, IgA2, IgE, IgM, or IgD antibodies, including chimeric antibodies; dimeric, trimeric and multimeric antibodies; bispecific antibodies; trispecific antibodies; multispecific antibodies; recombinant and engineered antibodies; and antibody fragments (e.g. Fab fragments).

The term “antibody fragment” as used herein refers to fragments of biological relevance, e.g. fragments that comprise the above mentioned antigen binding domain, i.e. contribute to antigen binding, e.g. form part of the antigen binding domain. Certain preferred fragments comprise a heavy chain variable region (V_{H} domain) and a light chain variable region (V_{L} domain) of the antibodies of the invention.

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

The term “antibody” is thus used to refer to any antibody-like molecule that has an immunoglobulin (Ig) antigen binding domain, and this term includes antibody fragments and formats that comprise an antigen binding domain including but not limited to Fab, Fab′, F(ab′)₂, single domain antibodies (DABs) e.g. camelid VHH nanobodies or V_{NAR}s or VH domains or VL domains, TandAbs dimer, Fv, scFv (single chain Fv), dsFv, ds-scFv, Fd, scFv/Fc^{KIH}, linear antibodies, minibodies, diabodies, bispecific antibody fragments, bibody, tribody (scFv-Fab fusions, bispecific or trispecific, respectively, including scFv/Fab-Fc, and scFv/Fab-Fc^{KIH}); sc-diabody; single chain bispecific diabody (scDb); kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”), DART-Fc; Duabody, CrossMab, DuetMab, DNL, small antibody mimetics.

In certain embodiments, the antibody (or antigen binding protein) of the present invention comprises all or a portion of a heavy chain constant region, such as an IgG1, IgG2, IgG3, IgG4, IgA1, IgA2, IgE, IgM, or IgD constant region. Preferably, the heavy chain constant region is an IgG heavy chain constant region or a portion thereof. IgG1 is a preferred format for the antibodies of the invention. Preferably antibodies of the invention comprise or contain human heavy chain constant regions.

Furthermore, the antibody or antigen binding protein of the invention can comprise all or a portion of a kappa light chain constant region or a lambda light chain constant region, or a portion thereof. Preferably antibodies of the invention comprise or contain human light-chain constant regions.

All or part of such constant regions may be produced naturally or may be wholly or partially synthetic. Appropriate sequences for such constant regions are well known and documented in the art. When a full complement of constant regions from the heavy and light chains are included in the antibodies (or antigen binding proteins) of the invention, such antibodies are typically referred to herein as “full length” antibodies or “whole” antibodies. In some embodiments such full length or whole antibodies are preferred. Thus, in some embodiments, antibodies or antibody fragments which are divalent (or bivalent) for human IL-6R are preferred, i.e. antibodies or fragments with two antigen binding domains that bind to human IL-6R. In other embodiments, a Fab format or fragment is preferred. In other embodiments, an scFv format or fragment is preferred. Thus, in some embodiments, antibodies or antibody fragments which are monovalent for human IL-6R are preferred, i.e. antibodies or fragments with one, or a single, antigen binding domain that binds to human IL-6R.

The antibodies or antigen binding proteins can be produced naturally or can be wholly or partially synthetically produced.

The antigen binding domains of the antibodies or antigen binding proteins of the invention generally comprise an antibody light chain variable region (VL) that comprises three CDR domains and an antibody heavy chain variable region (VH) that comprises three CDR domains.

However, it is well documented in the art that the presence of three CDRs from the light chain variable domain and three CDRs from the heavy chain variable domain of an antibody is not always necessary for antigen binding. Thus, constructs smaller than the above classical antigen binding domain are known to be effective.

For example, camelid VHH antibodies and other single domain antibodies comprising VH domains alone show that these domains can bind to antigen with acceptably high affinities. Thus, three CDRs (or even a single CDR) can effectively bind antigen and form an antigen binding domain.

Thus, although preferred antigen binding domains in the antigen binding proteins (e.g. antibodies) of the invention might comprise six CDR regions (three from a light chain and three from a heavy chain), antigen binding proteins (e.g. antibodies) with antigen binding domains with fewer than six CDR regions (e.g. 3 CDR regions) are encompassed by the invention. Antigen binding proteins (e.g. antibodies) with antigen binding domains with CDRs from only the heavy chain or light chain are also contemplated, e.g. VH or VL domain antibodies. Thus, the term “antibody” as used herein also refers to any antibody-like molecule that has an immunoglobulin (Ig) antigen binding domain, and this term includes antibody fragments and formats that comprise an antigen binding domain with fewer than six CDR regions (preferably 3 CDR regions), for example single chain antibodies (e.g. camelid or shark heavy chain antibodies (HCAb)) and single domain camelid VHH antibodies (e.g. nanobodies) or V_{NAR}s or VH domains or VL domains. Such single domain antibodies may have the advantageous property of enhanced brain tissue penetration which would benefit their use in the treatment of central nervous system (CNS) disorders (Bélanger et al. (2019), Antibodies (Basel), 8(2): 27). Discussion of various features of the antibodies of other aspects of the invention and preferred embodiments apply mutatis mutandis to this aspect of the invention.

Preferred light chain CDR regions for use in conjunction with the specified heavy chain CDR regions to form the antigen binding domain are described elsewhere herein. However, other light chain variable regions that comprise three CDRs for use in conjunction with the heavy chain variable regions of the invention are also contemplated. Appropriate light chain variable regions which can be used in combination with the heavy chain variable regions of the invention and which give rise to an antibody which binds to human IL-6R in accordance with the invention can be readily identified by a person skilled in the art.

For example, a heavy chain variable region of the invention can be combined with a single light chain variable region or a repertoire of light chain variable regions and the resulting antibodies tested for binding to human IL-6R.

If desired, similar methods could be used to identify alternative heavy chain variable regions for use in combination with preferred light chain variable regions of the invention.

The antibody, antigen binding protein and nucleic acid molecules of the invention are generally “isolated” or “purified” molecules insofar as they are distinguished from any such components that may be present in situ within a human or animal body or a tissue sample derived from a human or animal body. The sequences may, however, correspond to or be substantially homologous to sequences as found in a human or animal body. Thus, the term “isolated” or “purified” as used herein in reference to nucleic acid molecules or sequences and proteins or polypeptides, e.g. antigen binding proteins or antibodies, refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

Thus, when used in connection with a protein or polypeptide molecule such as light chain CDRs 1, 2 and 3, heavy chain CDRs 1, 2 and 3, light chain variable regions, heavy chain variable regions, and antibodies, antibody fragments or antigen binding proteins of the invention, including full length antibodies, the term “isolated” or “purified” typically refers to a protein substantially free of cellular material or other proteins from the source from which it is derived. In some embodiments, particularly where the protein is to be administered to humans or animals, such isolated or purified proteins are substantially free of culture medium when produced by recombinant techniques, or chemical precursors or other chemicals when chemically synthesized.

It can be noted that in embodiments the antibodies (or antigen binding proteins) of the invention do not occur in nature and are, in that respect, man-made constructs in that they do not correspond to molecules that occur naturally. For example, preferred antibodies can be engineered or recombinantly produced, or are experimentally induced to be produced in an animal species, e.g. by immunization. In other words, in embodiments the antibodies or binding proteins of the invention are non-native or not naturally occurring.

A person skilled in the art will appreciate that the proteins and polypeptides of the invention, such as the heavy and light chain CDRs, the heavy and light chain variable regions, antibodies, antibody fragments and antigen binding proteins, may be prepared in any of several ways well known and described in the art, but are most preferably prepared using recombinant methods.

Nucleic acid fragments encoding the heavy and/or light chain regions of the antibodies or antigen binding proteins of the invention, as appropriate, can be derived or produced by any appropriate method, e.g. by cloning or synthesis.

Once nucleic acid fragments encoding the heavy and/or light chain regions of the antibodies or antigen binding proteins of the invention have been obtained, these fragments can be further manipulated by standard recombinant DNA techniques for example to convert the variable region fragments into full length antigen binding protein (e.g. antibody) molecules with appropriate constant region domains, or into particular formats of antibody fragment discussed elsewhere herein, e.g. Fab fragments or scFv fragments. Typically, or as part of this further manipulation procedure, the nucleic acid fragments encoding the antibody (binding protein) molecules of the invention are generally incorporated into one or more appropriate expression vectors in order to facilitate production or manipulation of the antibodies (binding proteins) of the invention.

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates an expression vector (or one or more expression vectors), e.g. a recombinant expression vector, containing or comprising a nucleic acid molecule (e.g. one or more nucleic acid molecules) of the invention, or a fragment thereof, and the necessary regulatory sequences for the transcription and translation of the protein sequence encoded by the nucleic acid molecule of the invention. In other words, expression vectors comprising nucleic acid molecules of the invention, or encoding antibodies or antigen binding proteins of the invention are provided.

Expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, NY, 1989) and other laboratory textbooks.

Suitable host cells include a wide variety of eukaryotic host cells and prokaryotic cells, as will be well known to a person skilled in the art. For example, the proteins of the invention may be expressed in yeast cells or mammalian cells, for example HEK or CHO cells. In addition, if appropriate, for example if a Fab, scFv or DAB format is chosen, the proteins of the invention may be expressed in prokaryotic cells, e.g. bacterial cells such as Escherichia coli (E. coli). Cell-free expression systems might also be used.

The proteins of the invention may also be prepared by chemical synthesis using techniques well known in the chemistry of proteins such as solid phase synthesis.

A yet further aspect provides an expression construct or expression vector or expression system (e.g. a viral or bacterial or yeast, or mammalian, or other expression construct, vector or system), e.g. one or more expression constructs or expression vectors, comprising one or more of the nucleic acid fragments or segments or molecules of the invention. Preferably the expression constructs or vectors or systems are recombinant. Preferably said constructs or vectors or systems further comprise the necessary regulatory sequences for the transcription and translation of the protein sequence encoded by the nucleic acid molecule of the invention.

A yet further aspect provides a host cell (e.g. a mammalian or bacterial or yeast host cell) or virus, e.g. one or more host cells or viruses, comprising one or more expression constructs or expression vectors of the invention. Also provided are host cells (e.g. a mammalian or bacterial or yeast host cell) or viruses, e.g. one or more host cells or viruses, comprising one or more of the nucleic acid molecules of the invention. A host cell (e.g. a mammalian host cell or bacterial host cell, or yeast host cell) or virus expressing an antibody (or antigen binding protein) of the invention forms a yet further aspect.

A yet further aspect of the invention provides a method of producing (or manufacturing) an antibody (or antigen binding protein) of the present invention comprising a step of culturing the host cells of the invention. Preferred methods comprise the steps of (i) culturing a host cell comprising one or more of the nucleic acid molecules or one or more of the expression vectors of the invention under conditions suitable for the expression of the encoded antibody or binding protein; and optionally (ii) isolating or obtaining the antibody or binding protein from the host cell or from the growth medium/supernatant. Such methods of production (or manufacture) may also comprise a step of purification of the antibody or antigen binding protein product and/or formulating the antibody or antigen binding protein product into a composition, e.g. a pharmaceutical composition, including at least one additional component, such as a pharmaceutically acceptable carrier or excipient or diluent.

In embodiments when the antibody or antigen binding protein of the invention is made up of more than one polypeptide chain (e.g. Fab formats or whole antibodies), then all the polypeptides are preferably expressed in the host cell, either from the same or a different expression vector, so that the complete proteins, e.g. antibody proteins of the invention, can assemble in the host cell and be isolated or purified therefrom.

Compositions comprising at least a first antibody (or antigen binding protein) of the invention, or at least a first nucleic acid molecule or expression vector of the invention, or at least a first host cell of the invention, constitute further aspects of the present invention. Formulations (compositions) comprising one or more antibodies, etc., of the invention, optionally in admixture with a suitable diluent, carrier or excipient constitute a preferred embodiment of the present invention. Such formulations may be for pharmaceutical use, and thus compositions of the invention are preferably pharmaceutically acceptable or otherwise acceptable for administration to human or non-human animals, but in particular humans. Suitable diluents, excipients and carriers are known to the skilled person.

Any appropriate mode of administration can be used. The compositions according to the invention may be presented, for example, in a form suitable for oral, nasal, parenteral (e.g. intravenous, intraperitoneal, subcutaneous, intradermal, intramuscular), topical or rectal administration, or for mucosal delivery, and any of these modes of administration, or indeed any other appropriate mode of administration, can be used. In a preferred embodiment, compositions according to the invention are presented in a form suitable for intravenous administration. In some embodiments, compositions according to the invention are presented in a form suitable for intraperitoneal (i.p.) administration. In some embodiments, compositions according to the invention are presented in a form suitable for injection directly into a tumour (intratumoral).

The active compounds (e.g. the antibodies or antigen binding proteins of the invention) as defined herein may be presented in the conventional pharmacological forms of administration, such as tablets, coated tablets, nasal sprays, solutions, emulsions, liposomes, exosomes, powders, capsules or sustained release forms. Conventional pharmaceutical excipients as well as the usual methods of production may be employed for the preparation of these forms. In addition, nucleic acids or nucleic acid based vectors, e.g. mRNA based vectors, or virus-based vectors, may be used for administration of the active compounds of the invention, e.g. by encoding the antibodies or binding proteins of the invention.

Injection solutions may, for example, be produced in the conventional manner, such as by the addition of preservation agents, such as p-hydroxybenzoates, or stabilizers, such as EDTA. The solutions may then be filled into injection vials or ampoules.

The pharmaceutical compositions (formulations) of the present invention are preferably administered parenterally. Intravenous administration is preferred. In some embodiments, administration is intraperitoneal (i.p.) administration. In some embodiments, administration is by injection into a tumour. Parenteral administration may be performed by subcutaneous, intramuscular, intraperitoneal or intravenous injection by means of a syringe.

Alternatively, parenteral administration can be performed by means of an infusion pump. A further option is a composition which may be a powder or a liquid for the administration of the antibody or binding protein in the form of a nasal or pulmonal spray. As a still further option, the antibodies or binding proteins of the invention can also be administered transdermally, e.g. from a patch, optionally an iontophoretic patch, or transmucosally, e.g. bucally.

Suitable dosage units can be determined by a person skilled in the art.

A further aspect of the present invention provides the antibodies (or antigen binding proteins) or compositions of the invention for use in therapy, in particular for use in preventing or treating disease.

In some embodiments, the disease is linked to, caused by, characterized by or associated with, undesired, inappropriate, aberrant, pathological, pathogenic, increased or excessive IL-6 signalling or gp130 signalling induced by IL-6R/sIL-6R, preferably undesired, inappropriate, aberrant, pathological, pathogenic, increased or excessive IL-6 trans signalling.

Alternatively viewed, the disease is linked to, caused by, characterized by or associated with, undesired, inappropriate, aberrant, pathological, pathogenic, increased or excessive IL-6 signalling in cells not expressing the cognate IL-6R on the cell membrane (membrane-bound IL-6R).

In some embodiments, the disease is one or more of: cancer, arthritis for example rheumatoid arthritis or Systemic juvenile idiopathic arthritis, Castleman disease, Cytokine release syndrome, multiple sclerosis, anaemia, irritable bowel syndrome (IBS), inflammatory bowel disease (IBD) for example Crohn's disease or ulcerative colitis, pancreatitis, acute lung injury (ALI), depression, Alzheimer's disease, sepsis and Covid-19 or Covid-19 complications.

In some embodiments, the disease is an inflammatory disease, including an autoimmune disease, or cancer. In some embodiments, the disease involves pathological or pathogenic inflammation.

Thus, the antibodies (or antigen binding proteins) of the invention can be used to suppress inflammation or reduce inflammation. Types of inflammatory disease to be treated in accordance with the present invention include, but are not limited to, cancer, rheumatoid arthritis, Systemic juvenile idiopathic arthritis, Castleman disease, Cytokine release syndrome, multiple sclerosis, irritable bowel syndrome (IBS), inflammatory bowel disease (IBD) for example Crohn's disease or ulcerative colitis, pancreatitis, acute lung injury (ALI), Alzheimer's disease, sepsis and Covid-19 or Covid-19 complications.

Types of cancer to be treated in accordance with the present invention include, but are not limited to, colorectal cancer (i.e. colon cancer), liver cancer, lung cancer, colitis-associated cancer, pancreatic cancer, multiple myeloma, lymphoma, leukaemia and Hodgkin's disease.

In some embodiments, the disease is a central nervous system (CNS) or neurodegenerative disorder. Types of central nervous system (CNS) disorder to be treated in accordance with the present invention include, but are not limited to, multiple sclerosis, depression and Alzheimer's disease.

In other embodiments, the compositions, nucleic acid molecules, expression vectors, host cells or viruses of the invention can also be used in the therapeutic methods described herein.

The administration of the antibodies or antigen binding proteins in the therapeutic methods and uses of the invention is carried out in pharmaceutically, therapeutically, or physiologically effective amounts, to subjects (e.g. animals, e.g. human or non-human mammals) in need of treatment. Thus, said methods and uses may involve the additional step of identifying a subject in need of treatment. Appropriate and effective concentrations/doses to be administered can readily be determined by a person skilled in the art.

Treatment of diseases or conditions in accordance with the present invention (for example treatment of pre-existing disease) includes cure of said disease or condition, or any reduction or alleviation of disease, e.g. reduction in disease severity, or symptoms of disease.

The therapeutic methods and uses of the present invention are suitable for prevention of diseases as well as active treatment of diseases (for example treatment of pre-existing disease). Thus, prophylactic treatment is also encompassed by the invention. For this reason in the methods and uses of the present invention, treatment also includes prophylaxis, or prevention where appropriate.

Such preventative (or protective) aspects can conveniently be carried out on healthy or normal or at risk subjects and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

Suitable subjects for treatment in accordance with the present invention thus include humans suffering from, or at risk of suffering from, any of the diseases referred to above, including, inflammatory disease, a central nervous system (CNS) disorder, or cancer, and more specifically inflammatory diseases, central nervous system disorders and cancers associated with or caused by or characterised by undesired, inappropriate, aberrant, pathological, increased or excessive IL-6 signalling, preferably undesired, inappropriate, aberrant, pathological, increased or excessive IL-6 trans signalling.

Thus, the in vivo methods and uses as described herein are generally carried out in humans.

Thus, the term “patient” or “subject” as used herein preferably refers to humans.

In another embodiment, the subject is a subject having, or suspected of having (or developing), or potentially having (or developing) the disease or condition in question as described above.

In an alternative embodiment, the invention provides a method for treating or preventing a disease or disorder comprising administering to a patient in need thereof a therapeutically effective amount of an antibody (or antigen binding protein) of the present invention. Appropriate examples of diseases or disorders are defined above. Preferably the disease or disorder comprises one or more of: cancer, arthritis for example rheumatoid arthritis or Systemic juvenile idiopathic arthritis, Castleman disease, Cytokine release syndrome, multiple sclerosis, anaemia, irritable bowel syndrome (IBS), inflammatory bowel disease (IBD) for example Crohn's disease or ulcerative colitis, pancreatitis, acute lung injury (ALI), depression, Alzheimer's disease, sepsis and Covid-19 or Covid-19 complications. Embodiments of the therapeutic uses of the invention described herein apply, mutatis mutandis, to this aspect of the invention.

In an alternative embodiment, the invention provides a method for treating or preventing a disease or disorder that is linked to, caused by, characterized by or associated with, undesired, inappropriate, aberrant, pathological, pathogenic, increased or excessive IL-6 signalling or gp130 signalling induced by IL-6R/sIL-6R, preferably undesired, inappropriate, aberrant, pathological, pathogenic, increased or excessive IL-6 trans signalling, said method comprising administering to a patient in need thereof a therapeutically effective amount of an antibody (or antigen binding protein) of the present invention. Embodiments of the therapeutic uses of the invention described herein apply, mutatis mutandis, to this aspect of the invention.

In an alternative embodiment, the invention provides a method for treating or preventing a disease or disorder that is linked to, caused by, characterized by or associated with, undesired, inappropriate, aberrant, pathological, pathogenic, increased or excessive IL-6 signalling in cells not expressing the cognate IL-6R on the cell membrane (membrane-bound IL-6R), said method comprising administering to a patient in need thereof a therapeutically effective amount of an antibody (or antigen binding protein) of the present invention. Embodiments of the therapeutic uses of the invention described herein apply, mutatis mutandis, to this aspect of the invention.

By “therapeutically effective amount” is meant an amount sufficient to show benefit to the condition of the subject. Whether an amount is sufficient to show benefit to the condition of the subject may be determined by the subject him/herself or a physician; preferably it is determined by clinical assessment and can be readily monitored.

In an alternative embodiment, the invention provides the use of an antibody (or antigen binding protein) of the invention in the manufacture of a medicament for use in therapy, e.g. for use in the treatment or prevention of disease. Appropriate examples of diseases or disorders are defined above. Preferably said therapy is the treatment or prevention of one or more of: cancer, arthritis for example rheumatoid arthritis or Systemic juvenile idiopathic arthritis, Castleman disease, Cytokine release syndrome, multiple sclerosis, anaemia, irritable bowel syndrome (IBS), inflammatory bowel disease (IBD) for example Crohn's disease or ulcerative colitis, pancreatitis, acute lung injury (ALI), depression, Alzheimer's disease, sepsis and Covid-19 or Covid-19 complications.

In an alternative embodiment, the invention provides the use of antibodies (or antigen binding proteins) of the invention in the manufacture of a medicament for use in treating or preventing a disease or disorder that is linked to, caused by, characterized by or associated with, undesired, inappropriate, aberrant, pathological, pathogenic, increased or excessive IL-6 signalling or gp130 signalling induced by IL-6R/sIL-6R, preferably undesired, inappropriate, aberrant, pathological, pathogenic, increased or excessive IL-6 trans signalling. Embodiments of the therapeutic uses of the invention described herein apply, mutatis mutandis, to this aspect of the invention.

In an alternative embodiment, the invention provides the use of antibodies (or antigen binding proteins) of the invention in the manufacture of a medicament for use in treating or preventing a disease or disorder that is linked to, caused by, characterized by or associated with, undesired, inappropriate, aberrant, pathological, pathogenic, increased or excessive IL-6 signalling in cells not expressing the cognate IL-6R on the cell membrane (membrane-bound IL-6R). Embodiments of the therapeutic uses of the invention described herein apply, mutatis mutandis, to this aspect of the invention.

Further alternatively viewed, the present invention provides the use of an antigen binding protein (e.g. antibody) of the invention as defined herein for the treatment or prevention of one or more of the diseases as outlined above. Embodiments of the therapeutic uses of the invention described herein apply, mutatis mutandis, to this aspect of the invention.

In some embodiments, the antibodies (or binding proteins) of the invention can be used in monotherapy. In other embodiments they can be used in combination with other standard cancer therapeutics and therapeutics used in the treatment of inflammatory diseases or central nervous system (CNS) disorders.

The invention further includes kits comprising one or more of the antibodies (or antigen binding proteins) or compositions of the invention, or one or more of the nucleic acid molecules encoding the antibodies (or antigen binding proteins) of the invention, or one or more expression vectors comprising the nucleic acid sequences of the invention, or one or more host cells or viruses comprising the expression vectors or nucleic acid sequences of the invention. Preferably said kits are for use in the methods and uses as described herein, e.g. the therapeutic methods or in vitro or in vivo methods/assays as described herein. Preferably said kits comprise instructions for use of the kit components. Preferably said kits are for treating or preventing diseases or conditions as described elsewhere herein, and optionally comprise instructions for use of the kit components to treat or prevent such diseases or conditions.

The antibodies (or antigen binding proteins) of the invention as defined herein may also be used as molecular tools for in vitro or in vivo applications and assays, for example binding assays or diagnostic assays. As the antigen binding proteins (e.g. antibodies) comprise at least one antigen binding domain that binds to IL-6R, e.g. human IL-6R, these can be used in any assay where an IL-6R binding member is required.

Thus, yet further aspects of the invention provide a reagent that comprises an antibody (or antigen binding protein) of the invention as defined herein and the use of such antibodies (or antigen binding proteins) as molecular tools, for example in in vitro or in vivo assays, for example for the detection of IL-6R, e.g. human IL-6R, e.g. in a sample of interest.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “antibody”, as used herein, means “at least a first antibody”.

In addition, where the terms “comprise”, “comprises”, “has” or “having”, or other equivalent terms are used herein, then in some more specific embodiments, for example in the definition of the CDR or FR sequences herein, these terms include the term “consists of” or “consists essentially of”, or other equivalent terms.

The term “decrease” or “reduce” or “inhibit” or “limit” or “prevent” or “block” (or equivalent terms) as described herein includes any measurable decrease or reduction when compared with an appropriate control. Appropriate controls would readily be identified by a person skilled in the art and might include non-treated or placebo treated subjects or healthy subjects, or samples or assays where no antibody is present, or where a control antibody that does not bind human IL-6R is present, for example an isotype control antibody. Preferably the decrease or reduction or inhibition, etc., will be significant, for example clinically or statistically significant.

The term “increase” (or equivalent terms) as described herein includes any measurable increase or elevation when compared with an appropriate control. Appropriate controls would readily be identified by a person skilled in the art and might include non-treated or placebo treated subjects or healthy subjects, or samples or assays where no antibody is present, or where a control antibody that does not bind human IL-6R is present, for example an isotype control antibody. Preferably the increase will be significant, for example clinically or statistically significant.

Preferably such increases (and indeed other increases, improvements or positive effects as mentioned elsewhere herein) or such decreases (and indeed other decreases, reductions, inhibitions or negative effects as mentioned elsewhere herein) are measurable increases, decreases, etc., (as appropriate), more preferably they are significant increases, decreases, etc., preferably clinically significant or statistically significant increases, for example with a probability value of 50.05 or <0.05, when compared to an appropriate control level or value (e.g. compared to an untreated or placebo treated subject or compared to a healthy or normal subject, or the same subject before treatment, or compared to samples or assays where no antibody is present, or where a control antibody that does not bind human IL-6R is present, for example an isotype control antibody).

Methods of determining the statistical significance of differences between test groups of subjects or differences in levels of a particular parameter are well known and documented in the art. For example herein a decrease or increase in level of a particular parameter or a difference between test groups of subjects is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of 50.05 or <0.05. In all embodiments, reference to significant or non-significant differences is preferably a reference to statistically significant or non-significant differences.

### Sequences

TABLE A

| | | |
|---|---|---|
| SEQ ID | | |
| NO: | Description | Sequence |

C07 antibody

| | | |
|---|---|---|
| 1 | VH domain (nt) | gaggtgaagctggtggaatctgggggaggcttagtgcagcctggagggtccctgaaactct |
| | | cctgtgcagcctctggattcactttcagtagctatgccatgtcttgggttcgccagactgcagac |
| | | aagaggctggagttggtcgcaaacattaatagtaatggtggtagtacctattatccagacagt |
| | | gtgaagggccgattcaccatctccagagacaatgccaagaacaccctgttcctgcaaatga |
| | | gcagtctgaagtctgaggacacagccatgtattactgtgcaagagaaggttactacactatg |
| | | gactactggggtcaaggaacctcagtcaccgtctcctca |
| 2 | VL domain (nt) | gacattgtgctgacccaatctccagcttctttggctgtgtctccagggcagagggccaccatat |
| | | cctgcagagccagtgaaagtgttgatagttatggcaatagttttatgaactggtaccagcaga |
| | | aaccaggacagccacccaaactcctcatctatcttgcatccaacctagaatctggggtccct |
| | | gccaggttcagtggcagtgggtctaggacagacttcaccctcaccattgatcctgtggaggct |
| | | gatgatgctgcaacctattactgtcagcaaaataatgaggatccgtacacgttcggaggggg |
| | | gaccaagctggaaataaaa |
| 3 | VH domain (aa) | EVKLVESGGGLVQPGGSLKLSCAASGFTFSSYAMSWVRQTADK |
| | | RLELVANINSNGGSTYYPDSVKGRFTISRDNAKNTLFLQMSSLKS |
| | | EDTAMYYCAREGYYTMDYWGQGTSVTVSS |
| 4 | VL domain (aa) | DIVLTQSPASLAVSPGQRATISCRASESVDSYGNSFMNWYQQKP |
| | | GQPPKLLIYLASNLESGVPARFSGSGSRTDFTLTIDPVEADDAAT |
| | | YYCQQNNEDPYTFGGGTKLEIK |
| 5 | Heavy CDR1 | GFTFSSYA |
| 6 | Heavy CDR2 | INSNGGST |
| 7 | Heavy CDR3 | AREGYYTMDY |
| 8 | Light CDR1 | ESVDSYGNSF |
| 9 | Light CDR2 | LAS |
| 10 | Light CDR3 | QQNNEDPYT |
| 11 | Heavy FR1 | EVKLVESGGGLVQPGGSLKLSCAAS |
| 12 | Heavy FR2 | MSWVRQTADKRLELVAN |
| 13 | Heavy FR3 | YYPDSVKGRFTISRDNAKNTLFLQMSSLKSEDTAMYYC |
| 14 | Heavy FR4 | WGQGTSVTVSS |
| 15 | Light FR1 | DIVLTQSPASLAVSPGQRATISCRAS |
| 16 | Light FR2 | MNWYQQKPGQPPKLLIY |
| 17 | Light FR3 | NLESGVPARFSGSGSRTDFTLTIDPVEADDAATYYC |
| 18 | Light FR4 | FGGGTKLEIK |

TABLE B

| | | |
|---|---|---|
| SEQ ID | | |
| NO: | Description | Sequence |

C09 antibody

| | | |
|---|---|---|
| 19 | VH domain (nt) | gaggtgcagcttgttgagtctgggggagacttagtgcagcctggagggtccctgaaactctc |
| | | ctgtgcagcctctggattcactttcagtaactatggcatgtcttgggttcgccagactccagaca |
| | | agaggctggaattggtcgcagccattaatagtaatggtggtagcacctattatccagacagt |
| | | gtgaagggccgattcaccatctccagagacaatgccaagaacaccctgtacctgcaaatg |
| | | agcagtctgaagtctgaggacacagccatgtattactgtgcaagggacggtaactacgtatc |
| | | tgactactggggccaaggcaccactctcacagtctcctca |
| 20 | VL domain (nt) | gacattgtgctgacccaatctccagcttctttggctgtgtctctagggcagagggccaccatat |
| | | cctgcagagccagtgaaagtgttgatagttatggcaatagttttatgcactggtaccagcaga |
| | | aaccaggacagccacccaaactcctcatctatcttgcatccaacctagaatctggggtccct |
| | | gccaggttcagtggcagtgggtctaggacagacttcaccctcaccattgatcctgtggaggct |
| | | gatgatgctgcaacctattactgtcagcaaaataatgaggatccgtggacgttcggtggagg |
| | | caccaagctggaaatcaaa |
| 21 | VH domain (aa) | EVQLVESGGDLVQPGGSLKLSCAASGFTFSNYGMSWVRQTPDK |
| | | RLELVAAINSNGGSTYYPDSVKGRFTISRDNAKNTLYLQMSSLKS |
| | | EDTAMYYCARDGNYVSDYWGQGTTLTVSS |
| 22 | VL domain (aa) | DIVLTQSPASLAVSLGQRATISCRASESVDSYGNSFMHWYQQKP |
| | | GQPPKLLIYLASNLESGVPARFSGSGSRTDFTLTIDPVEADDAAT |
| | | YYCQQNNEDPWTFGGGTKLEIK |
| 23 | Heavy CDR1 | GFTFSNYG |
| 24 or 6 | Heavy CDR2 | INSNGGST |
| 25 | Heavy CDR3 | ARDGNYVSDY |
| 26 or 8 | Light CDR1 | ESVDSYGNSF |
| 27 or 9 | Light CDR2 | LAS |
| 28 | Light CDR3 | QQNNEDPWT |
| 29 | Heavy FR1 | EVQLVESGGDLVQPGGSLKLSCAAS |
| 30 | Heavy FR2 | MSWVRQTPDKRLELVAA |
| 31 | Heavy FR3 | YYPDSVKGRFTISRDNAKNTLYLQMSSLKSEDTAMYYC |
| 32 | Heavy FR4 | WGQGTTLTVSS |
| 33 | Light FR1 | DIVLTQSPASLAVSLGQRATISCRAS |
| 34 | Light FR2 | MHWYQQKPGQPPKLLIY |
| 35 or 17 | Light FR3 | NLESGVPARFSGSGSRTDFTLTIDPVEADDAATYYC |
| 36 or 18 | Light FR4 | FGGGTKLEIK |

TABLE C

| | | |
|---|---|---|
| SEQ ID | | |
| NO: | Description | Sequence |

C12 antibody

| | | |
|---|---|---|
| 37 | VH domain (nt) | gaggtgaagctgatggaatctggaggaggcttagtgcagcctggagggtccctgaaactct |
| | | cctgtgcagcctctggattcactttcagtaactatggcatgtcttgggttcgccagactccagac |
| | | aagaggctggaatgggtcgcaaacattaatagtaatggtgggagcacctattatccagaca |
| | | gtgtgaagggccgattcaccatctccagagacaatgcccagaacaccctgtatctgcaaat |
| | | gagcagtctgaagtctgaggacacagccatgtattactgtacaagagacggtaacttcgtat |
| | | ctgactactggggccaaggcaccactctcacagtctcctca |
| 38 or 2 | VL domain (nt) | gacattgtgctgacccaatctccagcttctttggctgtgtctccagggcagagggccaccatat |
| | | cctgcagagccagtgaaagtgttgatagttatggcaatagttttatgaactggtaccagcaga |
| | | aaccaggacagccacccaaactcctcatctatcttgcatccaacctagaatctggggtccct |
| | | gccaggttcagtggcagtgggtctaggacagacttcaccctcaccattgatcctgtggaggct |
| | | gatgatgctgcaacctattactgtcagcaaaataatgaggatccgtacacgttcggaggggg |
| | | gaccaagctggaaataaaa |
| 39 | VH domain (aa) | EVKLMESGGGLVQPGGSLKLSCAASGFTFSNYGMSWVRQTPDK |
| | | RLEWVANINSNGGSTYYPDSVKGRFTISRDNAQNTLYLQMSSLK |
| | | SEDTAMYYCTRDGNFVSDYWGQGTTLTVSS |
| 40 or 4 | VL domain (aa) | DIVLTQSPASLAVSPGQRATISCRASESVDSYGNSFMNWYQQKP |
| | | GQPPKLLIYLASNLESGVPARFSGSGSRTDFTLTIDPVEADDAAT |
| | | YYCQQNNEDPYTFGGGTKLEIK |
| 41 or 23 | Heavy CDR1 | GFTFSNYG |
| 42 or 6 | Heavy CDR2 | INSNGGST |
| 43 | Heavy CDR3 | TRDGNFVSDY |
| 44 or 8 | Light CDR1 | ESVDSYGNSF |
| 45 or 9 | Light CDR2 | LAS |
| 46 or 10 | Light CDR3 | QQNNEDPYT |
| 47 | Heavy FR1 | EVKLMESGGGLVQPGGSLKLSCAAS |
| 48 | Heavy FR2 | MSWVRQTPDKRLEWVAN |
| 49 | Heavy FR3 | YYPDSVKGRFTISRDNAQNTLYLQMSSLKSEDTAMYYC |
| 50 or 32 | Heavy FR4 | WGQGTTLTVSS |
| 51 or 15 | Light FR1 | DIVLTQSPASLAVSPGQRATISCRAS |
| 52 or 16 | Light FR2 | MNWYQQKPGQPPKLLIY |
| 53 or 17 | Light FR3 | NLESGVPARFSGSGSRTDFTLTIDPVEADDAATYYC |
| 54 or 18 | Light FR4 | FGGGTKLEIK |

TABLE D

| | | |
|---|---|---|
| SEQ ID | | |
| NO: | Description | Sequence |

C19 antibody

| | | |
|---|---|---|
| 55 | VH domain (nt) | gaggtgaagctgatggaatctgggggaggcttagtgcagcctggagggtccctgaaattgt |
| | | cctgtgcagcctctggattcactttcagtaactatggcatgtcttgggttcgccagactccagac |
| | | aagaggctggagttggtcgcaattatgaatagtaaaggtggtagcacctattatccagacag |
| | | tgtgatgggccgattcaccatctccagagacaatgccaaaaacaccctgtacctgcaaatg |
| | | agcagtctgaagtctgaggacacagccatgtattactgtgcaagagatggttactacactatg |
| | | gactactggggtcaaggaacctcagtcaccgtctcctca |
| 56 | VL domain (nt) | gacattgtgctgacccaatctccagcttctttggctgtgtctctagggcagagggccaccatat |
| | | cctgcagagccagtaaaagtgttgatagttttggcaatagttttatgcactggtaccagcaga |
| | | aaccaggacagtcacccagactcctcatcttccttgcatccaacctagaatctggggtccctg |
| | | ccagattcagtggcagtgggtctaggacagacttcaccctcaccattgatcctgtggaggctg |
| | | atgatgctgcaacctattactgtcagcaaaataatgaggatccatacacgttcggagggggg |
| | | accaagctggaaataaaa |
| 57 | VH domain (aa) | EVKLMESGGGLVQPGGSLKLSCAASGFTFSNYGMSWVRQTPDK |
| | | RLELVAIMNSKGGSTYYPDSVMGRFTISRDNAKNTLYLQMSSLKS |
| | | EDTAMYYCARDGYYTMDYWGQGTSVTVSS |
| 58 | VL domain (aa) | DIVLTQSPASLAVSLGQRATISCRASKSVDSFGNSFMHWYQQKP |
| | | GQSPRLLIFLASNLESGVPARFSGSGSRTDFTLTIDPVEADDAAT |
| | | YYCQQNNEDPYTFGGGTKLEIK |
| 59 or 23 | Heavy CDR1 | GFTFSNYG |
| 60 | Heavy CDR2 | MNSKGGST |
| 61 | Heavy CDR3 | ARDGYYTMDY |
| 62 | Light CDR1 | KSVDSFGNSF |
| 63 or 9 | Light CDR2 | LAS |
| 64 or 10 | Light CDR3 | QQNNEDPYT |
| 65 or 47 | Heavy FR1 | EVKLMESGGGLVQPGGSLKLSCAAS |
| 66 | Heavy FR2 | MSWVRQTPDKRLELVAI |
| 67 | Heavy FR3 | YYPDSVMGRFTISRDNAKNTLYLQMSSLKSEDTAMYYC |
| 68 or 14 | Heavy FR4 | WGQGTSVTVSS |
| 69 or 33 | Light FR1 | DIVLTQSPASLAVSLGQRATISCRAS |
| 70 | Light FR2 | MHWYQQKPGQSPRLLIF |
| 71 or 17 | Light FR3 | NLESGVPARFSGSGSRTDFTLTIDPVEADDAATYYC |
| 72 or 18 | Light FR4 | FGGGTKLEIK |

TABLE E

| | | |
|---|---|---|
| SEQ ID | | |
| NO: | Description | Sequence |

C20 antibody

| | | |
|---|---|---|
| 73 | VH domain (nt) | gaggtgaagctgatggaatctgggggaggcttagtgcagcctggagggtccctgaaactct |
| | | cctgtgcagcctctggattcactttcagtaactatggcatgtcttgggttcgccagactccagac |
| | | aagaggctggagttggtcgcaatcattaatagtaatggtggtagcacctattatccagacagt |
| | | gtgaagggccgattcaccatctccagagacaatgccaagaacaccctgtacctgcaaatg |
| | | agcagtctgaagtctgaggacacagccatatattactgtgcaagagacggtaacttcgtatct |
| | | gactactggggccaaggcaccactctcacagtctcctca |
| 74 | VL domain (nt) | gacattgtgctgacccaatctccagcttctttggctgtgtctctagggcagagggccaccatat |
| | | cctgcagagccagtgaaagtgttgatagttatggcaataggtttttgaactggtaccagcaga |
| | | aaccaggacagccacccaaactcctcatctatcttgcatccaacctagaatctggggtccct |
| | | gccaggttcagtggcagtgggtctaggacagacttcaccctcaccattgatcctgtggaggct |
| | | gatgatgctgcaacctattactgtcagcaaaataatgaggatccgtacacgttcggaggggg |
| | | gaccaagctggaaataaaa |
| 75 | VH domain (aa) | EVKLMESGGGLVQPGGSLKLSCAASGFTFSNYGMSWVRQTPDK |
| | | RLELVAIINSNGGSTYYPDSVKGRFTISRDNAKNTLYLQMSSLKSE |
| | | DTAIYYCARDGNFVSDYWGQGTTLTVSS |
| 76 | VL domain (aa) | DIVLTQSPASLAVSLGQRATISCRASESVDSYGNRFLNWYQQKP |
| | | GQPPKLLIYLASNLESGVPARFSGSGSRTDFTLTIDPVEADDAAT |
| | | YYCQQNNEDPYTFGGGTKLEIK |
| 77 or 23 | Heavy CDR1 | GFTFSNYG |
| 78 or 6 | Heavy CDR2 | INSNGGST |
| 79 | Heavy CDR3 | ARDGNFVSDY |
| 80 | Light CDR1 | ESVDSYGNRF |
| 81 or 9 | Light CDR2 | LAS |
| 82 or 10 | Light CDR3 | QQNNEDPYT |
| 83 or 47 | Heavy FR1 | EVKLMESGGGLVQPGGSLKLSCAAS |
| 84 or 66 | Heavy FR2 | MSWVRQTPDKRLELVAI |
| 85 | Heavy FR3 | YYPDSVKGRFTISRDNAKNTLYLQMSSLKSEDTAIYYC |
| 86 or 32 | Heavy FR4 | WGQGTTLTVSS |
| 87 or 33 | Light FR1 | DIVLTQSPASLAVSLGQRATISCRAS |
| 88 | Light FR2 | LNWYQQKPGQPPKLLIY |
| 89 or 17 | Light FR3 | NLESGVPARFSGSGSRTDFTLTIDPVEADDAATYYC |
| 90 or 18 | Light FR4 | FGGGTKLEIK |

TABLE F
Consensus amino acid sequences for antibodies of the invention

| SEQ ID | | |
|---|---|---|
| NO: | Description | Sequence |
| 91 | Heavy CDR1 | GFTFSX₆YX₈, wherein X₆ and X₈ are any amino acid. |
| 92 | Heavy CDR1 | GFTFSX₆YX₈, wherein X₆ is N or S; and/or X₈ is G or A. |
| 93 | Heavy CDR1 | GFTFS[N/S]Y[G/A] |
| 94 | Heavy CDR2 | X₁NSX₄GGST, wherein X₁ and X₄ are any amino acid. |
| 95 | Heavy CDR2 | X₁NSX₄GGST, wherein X₁ is I or M; and/or X₄ is N or K. |
| 96 | Heavy CDR2 | [I/M]NS[N/K]GGST |
| 97 | Heavy CDR3 | X₁RX₃GX₅X₆X₇X₈DY, wherein X₁, X₃, X₅, X₆, X₇ and X₈ are any |
| | | amino acid. |
| | Heavy CDR3 | X₁RX₃GX₅X₆X₇X₈DY, wherein X₁ is A or T; X₃ is D or E; X₅ is N or Y; |
| | | X₆ is Y or F; X₇ is V or T; and/or X₈ is S or M. |
| 99 | Heavy CDR3 | [A/T]R[D/E]G[N/Y][Y/F][V/T][S/M]DY |
| 100 | Heavy CDR3 | ARX₃GYYTMDY, wherein X₃ is any amino acid. |
| 101 | Heavy CDR3 | ARX₃GYYTMDY, wherein X₃ is E or D. |
| 102 | Heavy CDR3 | AR[E/D]GYYTMDY |
| 103 | Heavy CDR3 | X₁RDGNX₆VSDY, wherein X₁ and X₆ are any amino acid. |
| 104 | Heavy CDR3 | X₁RDGNX₆VSDY, wherein X₁ is A or T; and/or X₆ is F or Y. |
| 105 | Heavy CDR3 | [A/T]RDGN[F/Y]VSDY |
| 106 | Light CDR1 | X₁SVDSX₆GNX₉F, wherein X₁, X₆ and X₉ are any amino acid. |
| 107 | Light CDR1 | X₁SVDSX₆GNX₉F, wherein X₁ is E or K; X₆ is Y or F; and/or X₉ is S |
| | | or R. |
| 108 | Light CDR1 | [E/K]SVDS[Y/F]GN[S/R]F |
| 109 | Light CDR3 | QQNNEDPX₈T, wherein X₈ is any amino acid. |
| 110 | Light CDR3 | QQNNEDPX₈T, wherein X₈ is Y or W. |
| 111 | Light CDR3 | QQNNEDP[Y/W]T |

Amino acid sequence of human membrane-bound IL-6R subunit alpha (Uniprot entry
P08887) (SEQ ID NO: 112):
MLAVGCALLAALLAAPGAALAPRRCPAQEVARGVLTSLPGDSVTLTCPGVEPEDNATVHW
VLRKPAAGSHPSRWAGMGRRLLLRSVQLHDSGNYSCYRAGRPAGTVHLLVDVPPEEPQL
SCFRKSPLSNVVCEWGPRSTPSLTTKAVLLVRKFQNSPAEDFQEPCQYSQESQKFSCQLA
VPEGDSSFYIVSMCVASSVGSKFSKTQTFQGCGILQPDPPANITVTAVARNPRWLSVTWQD
PHSWNSSFYRLRFELRYRAERSKTFTTWMVKDLQHHCVIHDAWSGLRHVVQLRAQEEFG
QGEWSEWSPEAMGTPWTESRSPPAENEVSTPMQALTTNKDDDNILFRDSANATSLPVQD
SSSVPLPTFLVAGGSLAFGTLLCIAIVLRFKKTWKLRALKEGKTSMHPPYSLGQLVPERPRP
TPVLVPLISP PVSPSSLGSDNTSSHNRPDARDPRSPYDISNTDYFFPR
Amino acid sequence of human soluble IL-6R (SEQ ID NO: 113):
MLAVGCALLAALLAAPGAALAPRRCPAQEVARGVLTSLPGDSVTLTCPGVEPEDNATVHW
VLRKPAAGSHPSRWAGMGRRLLLRSVQLHDSGNYSCYRAGRPAGTVHLLVDVPPEEPQL
SCFRKSPLSNVVCEWGPRSTPSLTTKAVLLVRKFQNSPAEDFQEPCQYSQESQKFSCQLA
VPEGDSSFYIVSMCVASSVGSKFSKTQTFQGCGILQPDPPANITVTAVARNPRWLSVTWQD
PHSWNSSFYRLRFELRYRAERSKTFTTWMVKDLQHHCVIHDAWSGLRHVVQLRAQEEFG
QGEWSEWSPEAMGTPWTESRSPPAENEVSTPMQALTTNKDDDNILFRDSANATSLPVQ
Amino acid sequence of human membrane bound IL-6R subunit beta (gp130) (Uniprot
entry P40189) (SEQ ID NO: 114):
MLTLQTWLVQALFIFLTTESTGELLDPCGYISPESPVVQLHSNFTAVCVLKEKCMDYFHVNA
NYIVWKTNHFTIPKEQYTIINRTASSVTFTDIASLNIQLTCNILTFGQLEQNVYGITIISGLPPEK
PKNLSCIVNEGKKMRCEWDGGRETHLETNFTLKSEWATHKFADCKAKRDTPTSCTVDYST
VYFVNIEVWVEAENALGKVTSDHINFDPVYKVKPNPPHNLSVINSEELSSILKLTWTNPSIKS
VIILKYNIQYRTKDASTWSQIPPEDTASTRSSFTVQDLKPFTEYVFRIRCMKEDGKGYWSDW
SEEASGITYEDRPSKAPSFWYKIDPSHTQGYRTVQLVWKTLPPFEANGKILDYEVTLTRWK
SHLQNYTVNATKLTVNLTNDRYLATLTVRNLVGKSDAAVLTIPACDFQATHPVMDLKAFPKD
NMLWVEWTTPRESVKKYILEWCVLSDKAPCITDWQQEDGTVHRTYLRGNLAESKCYLITVT
PVYADGPGSPESIKAYLKQAPPSKGPTVRTKKVGKNEAVLEWDQLPVDVQNGFIRNYTIFY
RTIIGNETAVNVDSSHTEYTLSSLTSDTLYMVRMAAYTDEGGKDGPEFTFTTPKFAQGEIEAI
VVPVCLAFLLTTLLGVLFCFNKRDLIKKHIWPNVPDPSKSHIAQWSPHTPPRHNFNSKDQMY
SDGNFTDVSVVEIEANDKKPFPEDLKSLDLFKKEKINTEGHSSGIGGSSCMSSSRPSISSSD
ENESSQNTSSTVQYSTVVHSGYRHQVPSVQVFSRSESTQPLLDSEERPEDLQLVDHVDGG
DGILPRQQYFKQNCSQHESSPDISHFERSKQVSSVNEEDFVRLKQQISDHISQSCGSGQMK
MFQEVSAADAFGPGTEGQVERFETVGMEAATDEGMPKSYLPQTVRQGGYMPQ
Amino acid sequence of mouse membrane-bound IL-6R subunit alpha (Uniprot entry
P22272) (SEQ ID NO: 115):
MLTVGCTLLVALLAAPAVALVLGSCRALEVANGTVTSLPGATVTLICPGKEAAGNVTIHWVY
SGSQNREWTTTGNTLVLRDVQLSDTGDYLCSLNDHLVGTVPLLVDVPPEEPKLSCFRKNPL
VNAICEWRPSSTPSPTTKAVLFAKKINTTNGKSDFQVPCQYSQQLKSFSCQVEILEGDKVYH
IVSLCVANSVGSKSSHNEAFHSLKMVQPDPPANLVVSAIPGRPRWLKVSWQHPETWDPSY
YLLQFQLRYRPVWSKEFTVLLLPVAQYQCVIHDALRGVKHVVQVRGKEELDLGQWSEWSP
EVTGTPWIAEPRTTPAGILWNPTQVSVEDSANHEDQYESSTEATSVLAPVQESSSMSLPTF
LVAGGSLAFGLLLCVFIILRLKQKWKSEAEKESKTTSPPPPPYSLGPLKPTFLLVPLLTPHSS
GSDNTVNHSCLGVRDAQSPYDNSNRDYLFPR
Amino acid sequence of mouse soluble IL-6R (SEQ ID NO: 116):
LVLGSCRALEVANGTVTSLPGATVTLICPGKEAAGNVTIHWVYSGSQNREWTTTGNTLVLR
DVQLSDTGDYLCSLNDHLVGTVPLLVDVPPEEPKLSCFRKNPLVNAICEWRPSSTPSPTTK
AVLFAKKINTTNGKSDFQVPCQYSQQLKSFSCQVEILEGDKVYHIVSLCVANSVGSKSSHNE
AFHSLKMVQPDPPANLVVSAIPGRPRWLKVSWQHPETWDPSYYLLQFQLRYRPVWSKEFT
VLLLPVAQYQCVIHDALRGVKHVVQVRGKEELDLGQWSEWSPEVTGTPWIAEPRTTPAGIL
WNPTQVSVEDSANHEDQYESSTE

The invention will now be further described in the following non-limiting Examples with reference to the following drawings:

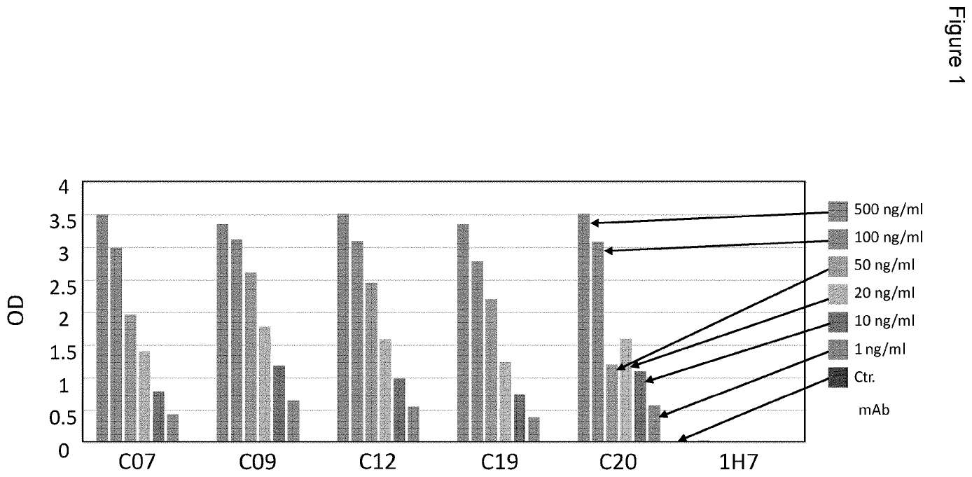

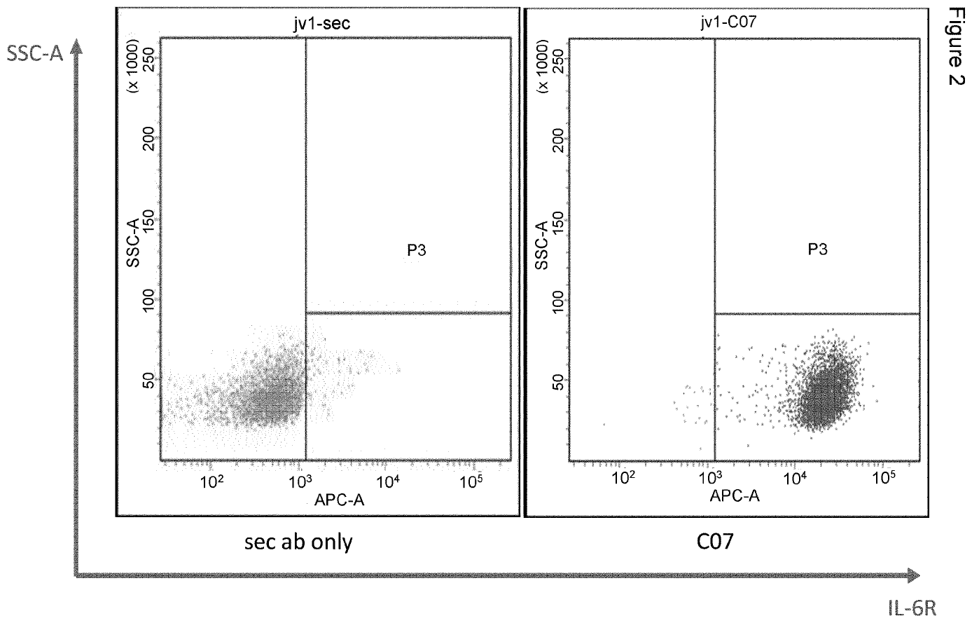

FIG. 1. ELISA assay to analyse binding of increasing concentrations of anti-IL-6R IgG antibodies (clones 07, 09, 12, 19 and 20) to sIL-6R. Anti-Mesothelin IgG antibody (clone 1H7) was used as a negative control.

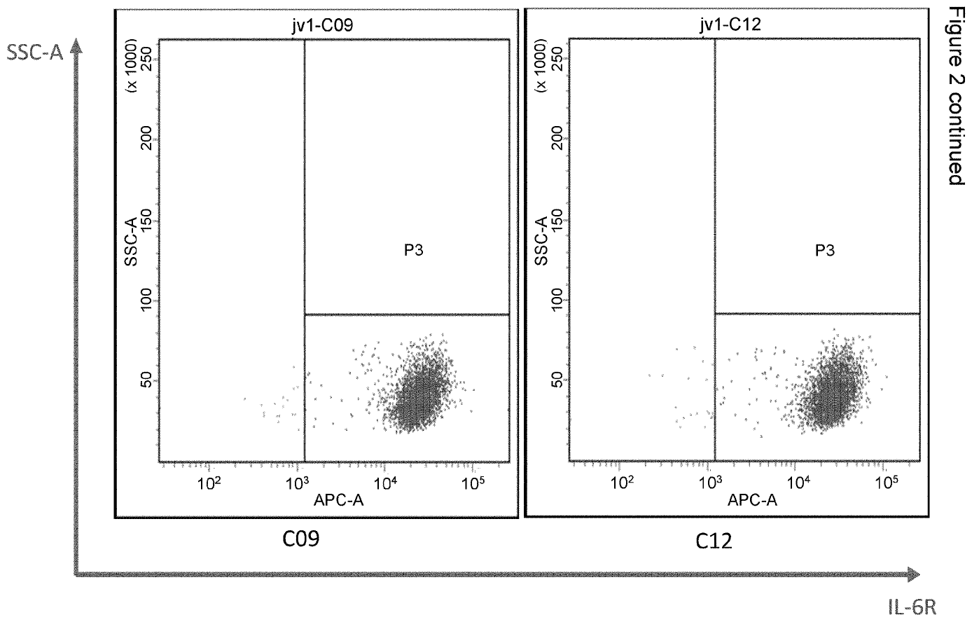

FIG. 2. Flow cytometry analysis of THP-1 cells that have been membrane-stained with the indicated anti-IL-6R IgG antibodies (clones 07, 09, 12, 19 and 20) and APC-labeled secondary antibodies. Cells stained only with APC-coupled secondary antibodies were used as control for unspecific binding of secondary antibodies.

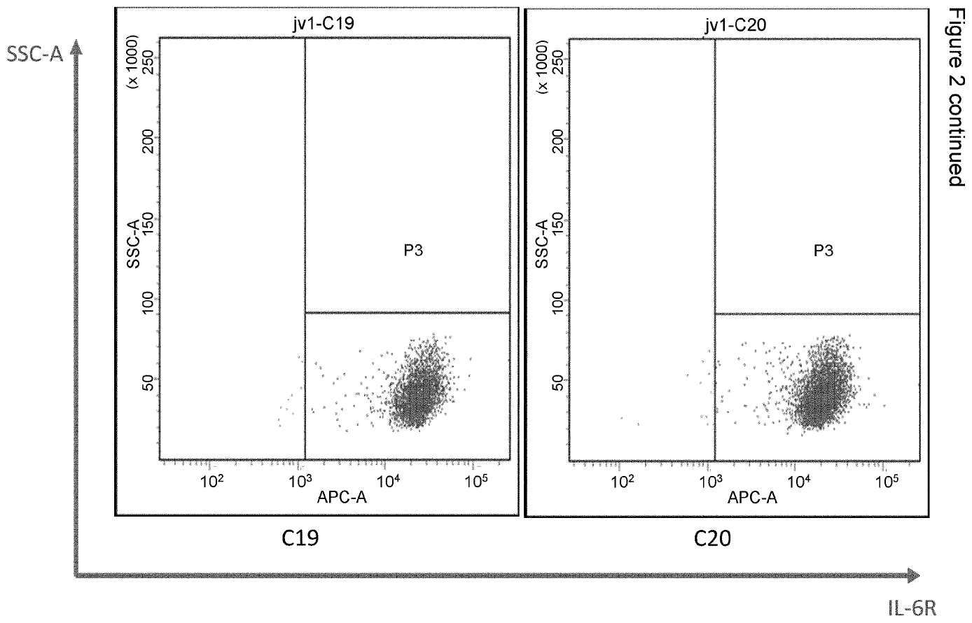

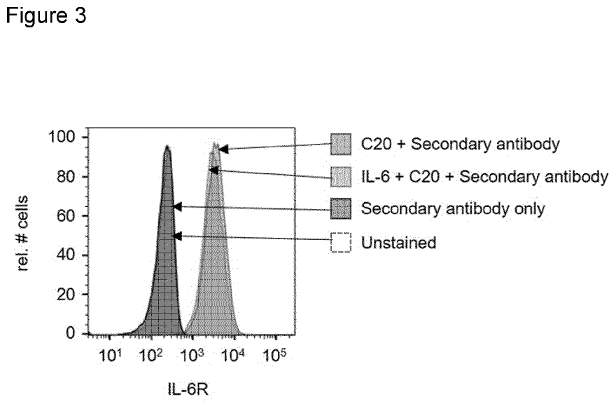

FIG. 3. Flow cytometry analysis of Ba/F3_gp130_IL-6R cells, incubated with and without IL-6, that have been membrane-stained with the anti-IL-6R IgG C20 antibody and PE-labeled secondary antibodies, presented as a histogram of the relative number of cells (rel. #cells) against PE fluorescence (IL-6R). Cells stained only with PE-coupled secondary antibodies were used as control for unspecific binding of secondary antibodies.

FIG. 4. Analysis of IL-6 trans signalling-induced proliferation of Ba/F3_gp130 cells cultivated with IL-6 (10 ng/ml), sIL-6R (200 ng/ml) and the indicated amounts of anti-IL-6R IgG antibodies (clones 07, 09, 12, 19 and 20) or anti-Mesothelin IgG (11H7). Proliferation in response to IL-6 and sIL-6R was set to 100%. Means±SD from n=3 independent experiments are shown. Data is also presented in Table 5.

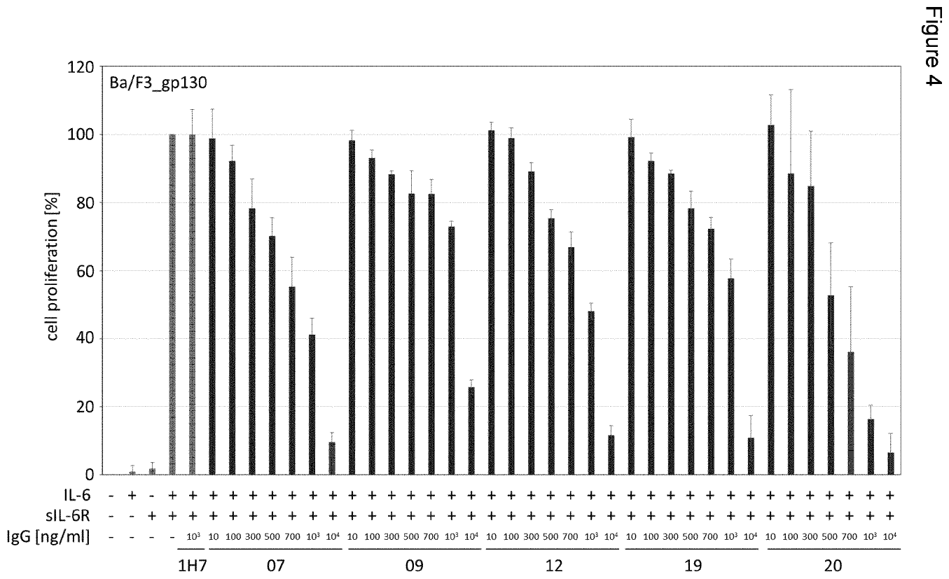

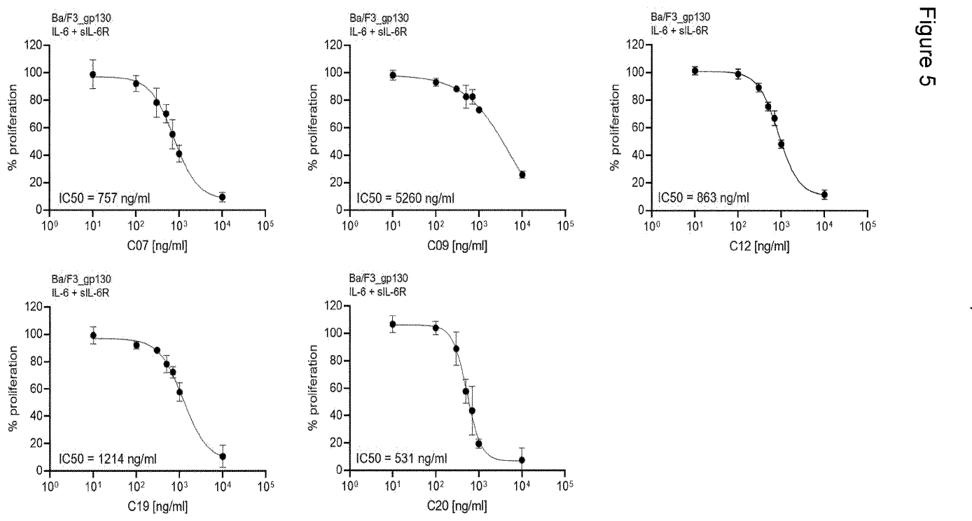

FIG. 5. Dose dependency curves for the inhibition of IL-6 trans signalling-induced cell proliferation of Ba/F3_gp130 cells cultivated with IL-6 (10 ng/ml) and sIL-6R (200 ng/ml) by the anti-IL-6R IgG antibodies (clones 07, 09, 12, 19 and 20). Data points were plotted from the mean values in Table 5 and Table 6. The IC50 values for the inhibition of IL-6 trans signalling-induced cell proliferation are also shown.

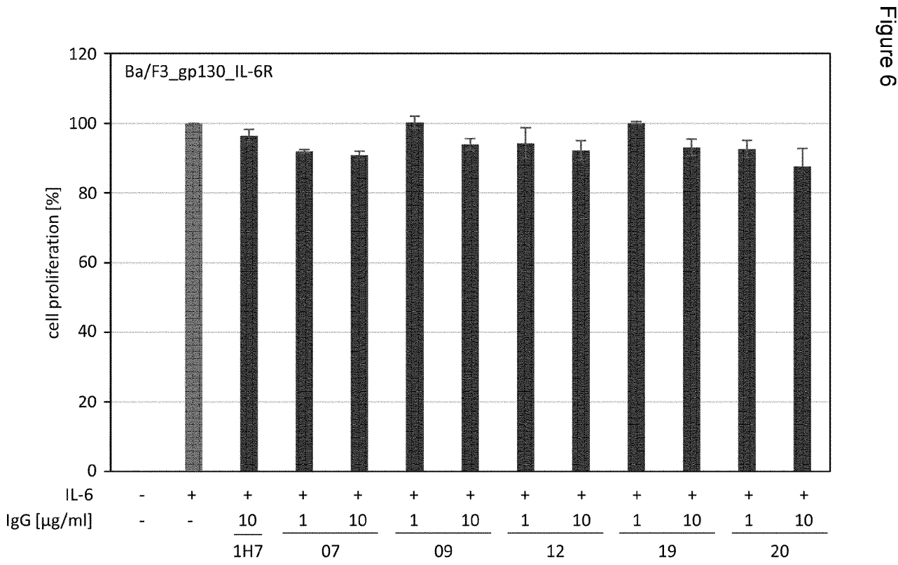

FIG. 6. Analysis of IL-6 classical signalling-induced proliferation of Ba/F3_gp130_IL-6R cells cultivated with IL-6 (10 ng/ml) and the indicated amounts of anti-IL-6R IgG antibodies (clones 07, 09, 12, 19 and 20) or anti-Mesothelin IgG (1H7). Proliferation in response to IL-6 was set to 100%. Means±SD from n=3 independent experiments are shown. Data is also presented in Table 9.

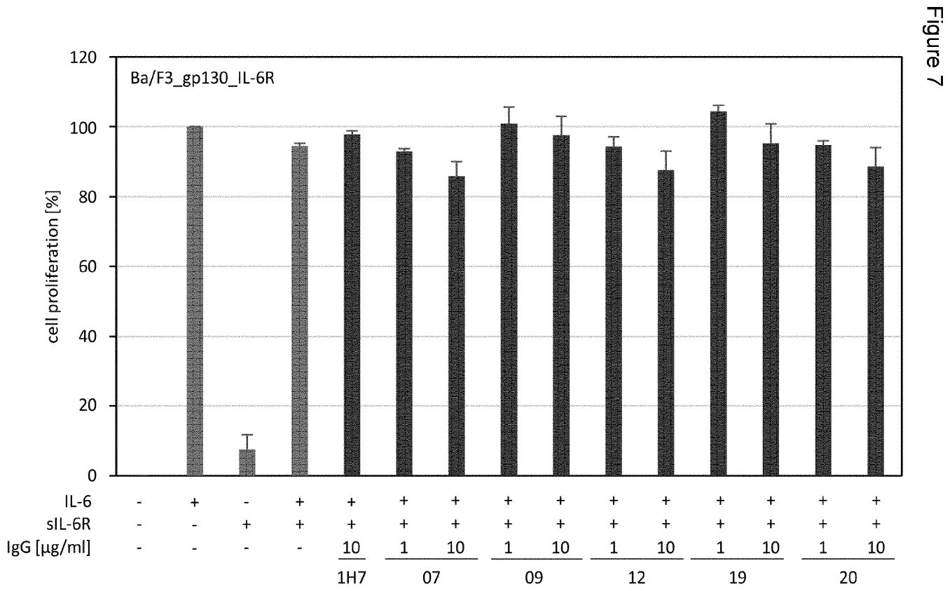

FIG. 7. Analysis of combined IL-6 classical and trans signalling-induced proliferation of Ba/F3_gp130_IL-6R cells cultivated with IL-6 (10 ng/ml), sIL-6R (200 ng/ml), and the indicated amounts of anti-IL-6R IgG antibodies (clones 07, 09, 12, 19 and 20) or anti-Mesothelin IgG (1H7). Proliferation in response to IL-6 was set to 100%. Means±SD from n=3 independent experiments are shown. Data is also presented in Table 11.

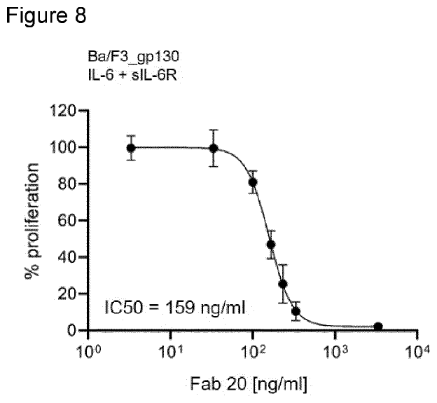

FIG. 8. Dose dependency curve for the inhibition of IL-6 trans signalling-induced cell proliferation of Ba/F3_gp130 cells cultivated with IL-6 (10 ng/ml) and sIL-6R (200 ng/ml) by the anti-IL-6R Fab C20 (3.3 ng/ml, 33.3 ng/ml, 100 ng/ml, 167 ng/ml, 233 ng/ml, 333 ng/ml and 3300 ng/ml). The IC50 for the inhibition of IL-6 trans signalling-induced cell proliferation is also shown.

### EXAMPLES

### Example 1—Production and Functional Characterisation of Fabs

### Materials and Methods

### Generation of Fabs Using Subtractive Immunization Protocol

Antibodies were identified by a subtractive immunization protocol, a technique which is for example described in Sandrock, 1987, J. Immunol Methods, 100:73-82. Specifically, in this case this protocol involves mice firstly being given four tolerizing cycles of IP injections with JV1 cells (which are Ba/F3 cells (ATCC) stably expressing human membrane-bound IL-6R) followed by cyclophosphamide treatment, and secondly being given four IP immunizing injections with recombinant human soluble IL-6R alpha. Following this subtractive immunization protocol the mice were sacrificed and RNA from the spleen cells of each mouse were used to generate a Fab phage display library for screening. Fabs were produced from bacterial cultures in which the bacteria were grown in 2TY medium+ampicillin+glucose and addition of IPTG (IsoPropyl β-D-1-ThioGalactopyranoside). 27 candidate Fabs were obtained for screening through an initial screening process.

### Cell Proliferation Assay to Analyse Inhibition of IL-6 Signalling Pathways by Fabs

As a cellular model for cell proliferation, two variants of a murine pre-B cell line (Ba/F3) were used. Ba/F3 wildtype cells do not express gp130 or IL-6R.
1) Ba/F3 cells stably expressing human gp130 (Ba/F3_gp130) (Gaering et al, PNAS, 1994, 91(3): 1119-11123). These cells do not proliferate in response to IL-6 classical signalling because they lack the membrane-bound IL-6R. Proliferation of Ba/F3_gp130 cells is induced by a complex of IL-6 and soluble IL-6R. Consequently, Ba/F3_gp130 cells serve as model for IL-6 trans signalling.
2) Ba/F3_gp130_IL-6R cells (Vollmer et al, Eur J Biochem, 1999, 263(2): 438-446) stably express both human membrane-bound gp130 and membrane-bound IL-6R. These cells proliferate in response to stimulation with IL-6 and therefore serve as a model for IL-6 classical signalling. When stimulated with IL-6 and soluble IL-6R, both IL-6 classical and trans signalling pathways (IL-6 mixed signaling) are activated in Ba/F3_gp130_IL-6R cells.

To analyse cytokine-induced cell proliferation mediated by the IL-6 trans or combined IL-6 classical and trans (IL-6 mixed) pathways, 5,000 cells/well were seeded in 96-well plates in DMEM (10% FCS+penicillin/streptomycin). Cells were cultured in the presence of human IL-6 (Peprotech, 10 ng/ml) and human soluble IL-6R (200 ng/ml, SEQ ID NO:113) to stimulate IL-6 trans signalling in Ba/F3_gp130 cells and stimulate combined IL-6 classical and trans signalling (mixed signalling) in Ba/F3_gp130_IL-6R cells. To determine the inhibitory potential of the candidate Fabs, 1% periplasmic extracts (PEs) containing each of the 27 Fabs were added. PE containing an anti-Mesothelin Fab (clone 1H7, MAB Designs) was used as control for unspecific growth inhibition by PE. After 48 h the number of viable cells was determined using Cell Titer Blue reagent (Promega) according to the manufacturer's instructions. Absorption at 570 nm and 600 nm was recorded. For normalization, the absorbance of cells grown without cytokine was subtracted from absorbance of each sample. The absorbance of cells grown with cytokines was set to 100%.

### Intracellular Signalling Assay to Analyse Inhibition of IL-6 Signalling Pathways by Fabs

Two variants of human embryonic kidney cells (HEK T-REx™-293, ThermoFisher) were used as a cellular model for intracellular signalling.
1) HEK293 cells endogenously express membrane-bound gp130 but do not express membrane-bound IL-6R. These cells do not proliferate in response to IL-6 classical signalling because they lack the membrane-bound IL-6R. Proliferation of HEK293 cells is induced by a complex of IL-6 and soluble IL-6R. They therefore serve as model for IL-6 trans signalling.
2) HEK293_IL-6R were derived from HEK293 cells by stable transduction of human IL-6R (Dittrich et al. Mol Biosyst. 2012. 8(8):2119-2134). When stimulated with IL-6, IL-6 classical signalling is activated. When stimulated with IL-6 and soluble IL-6R, both IL-6 classical and trans signalling pathways (IL-6 mixed signalling) are activated in HEK293_IL-6R cells.

To analyse cytokine-induced intracellular signalling mediated by the IL-6 trans or combined IL-6 classical and trans (IL-6 mixed) pathways, IL-6-induced STAT3 Y705 phosphorylation was determined by intracellular flow cytometry. 2*10⁵ cells/well were seeded in 12-well dishes in DMEM (10% FCS+penicillin/streptomycin) (HEK293) or DMEM (10% FCS+penicillin/streptomycin+2 μg puromycin) (HEK293_IL-6R). Cells were incubated at 37° C. and 5% CO₂ in a water saturated atmosphere. After 48 h, cells were starved in 900 μl DMEM without FCS and antibiotics for 2 h. Cells were then treated with IL-6 (10 ng/ml) and soluble IL-6R (200 ng/ml) to stimulate IL-6 trans signalling in HEK293 cells and stimulate combined IL-6 classical and trans signalling (IL-6 mixed signalling) in HEK293_IL-6R cells. To determine the inhibitory potential of the candidate Fabs, 1% PEs containing each of the 27 Fabs was added for 30 mins. After stimulation, the medium was removed and 150 μl Trypsin-EDTA per well was added. 100 μl cell suspension was transferred to individual wells of a 96-well plate, pre-filled with 100 μl 4% paraformaldehyde. Cells were fixed for 10 min at 37° C. After fixation, the plate was centrifuged (5 mins, 300 g, 4° C.). The supernatant was removed and cells were resuspended in 200 μl ice-cold 90% methanol. The plate was incubated for 10 min at −20° C. and then centrifuged. Cells were washed two times with 200 μl 1% BSA, 0.5 mM EDTA in PBS (FACS buffer). Cells were resuspended in 50 μl antibody solution (anti (p)Y STAT3, clone: 4/P-STAT3 (BD Biosciences), 1:200 in FACS buffer) and incubated overnight at 4° C. in the dark. The cells were washed three times with FACS buffer. Finally, fluorescence was determined using a flow cytometer. For normalization, fluorescence of unstimulated cells was subtracted from each sample. Fluorescence of cells stimulated with cytokines was set to 100%.

### Results

### Selective Inhibition of IL-6 Trans Signalling-Induced Cell Proliferation by Fabs

In an initial screening process, the Fabs most likely to be selective for the inhibition of IL-6 trans signalling were identified from the 27 candidate Fabs. To this end, it was tested whether PE, each containing one of the 27 Fabs, selectively inhibit IL-6 trans signalling compared to IL-6 classical signalling. As a readout, IL-6 trans and classical signalling induced cell proliferation was analysed.

First, it was tested which of the 27 candidate Fabs inhibit IL-6 trans signalling-induced proliferation. For this purpose, Ba/F3_gp130 cells were stimulated with IL-6 and soluble IL-6R and the inhibitory capacity of the Fab containing PEs was tested (Table 1). The control PE containing anti-Mesothelin Fab does not reduce proliferation of Ba/F3_gp130 cells, suggesting that PE alone does not block the proliferation of these cells. In contrast, all 27 candidate Fabs inhibit IL-6 trans signalling-induced proliferation. The strength of the inhibition varies from clone to clone. For example, while clones 01, 20, and 26 reduce IL-6 trans signalling-induced proliferation by more than 50%, clones 08 and 23 reduce proliferation only slightly.

TABLE 1
Inhibition of IL-6 trans signalling-induced proliferation by
PE containing each of the 27 candidate Fabs. Proliferation of
Ba/F3_gp130 cells induced by 10 ng/ml IL-6 and 200 ng/ml
soluble IL-6R (sIL-6R) was set to 100%. Results are given
as mean fromn = 3 independent experiments ± SD.
Ba/F3_gp130

| | | | Proliferation in % | |
|---|---|---|---|---|
| Sample | | IL-6:sIL-6R | Mean | SD |
| Ctr. | | − | 0.0 | 0.0 |
| IL-6:sIL-6R | | + | 100.0 | 0.0 |
| Periplasmic | 1H7 | + | 96.3 | 0.2 |
| extracts | 01 | + | 44.1 | 26.3 |
| containing | 02 | + | 69.4 | 27.8 |
| Fabs | 03 | + | 60.9 | 20.8 |
| | 04 | + | 66.0 | 23.8 |
| | 05 | + | 67.4 | 16.6 |
| | 06 | + | 58.6 | 16.4 |
| | 07 | + | 55.7 | 9.2 |
| | 08 | + | 89.9 | 20.4 |
| | 09 | + | 69.8 | 24.1 |
| | 10 | + | 61.8 | 21.5 |
| | 11 | + | 76.2 | 22.7 |
| | 12 | + | 47.9 | 16.9 |
| | 13 | + | 67.3 | 14.6 |
| | 14 | + | 60.5 | 16.7 |
| | 15 | + | 66.5 | 14.7 |
| | 16 | + | 48.5 | 10.5 |
| | 17 | + | 73.8 | 22.9 |
| | 18 | + | 59.5 | 19.2 |
| | 19 | + | 53.3 | 19.5 |
| | 20 | + | 36.6 | 13.4 |
| | 21 | + | 58.6 | 14.5 |
| | 22 | + | 58.3 | 19.9 |
| | 23 | + | 84.0 | 20.3 |
| | 24 | + | 75.7 | 16.4 |
| | 25 | + | 57.6 | 22.2 |
| | 26 | + | 34.7 | 22.1 |
| | 27 | + | 51.0 | 18.2 |

To address whether the observed inhibition of IL-6-induced proliferation is selective for IL-6 trans signalling, it was next tested whether IL-6 classical signalling-induced proliferation is also inhibited by the 27 candidate Fabs. In the body, cells are confronted with both IL-6 and soluble IL-6R, allowing classical and trans-signalling in cells expressing membrane-bound IL-6R (combined classical and trans signalling, termed IL-6 mixed signalling). However, high expression of membrane-bound IL-6R favors classical signalling (Reeh et al. Cell Comm Signal. 2019. 17(1):46). Therefore, stimulation of cells expressing high numbers of IL-6R allows us to approximate the ability of the Fabs to reduce IL-6 classical signalling under physiological conditions. Ba/F3_gp130_IL-6R can be stimulated by membrane-bound IL-6R and soluble IL-6R and thus fulfil this prerequisite (Reeh et al., 2019, supra).

Ba/F3_gp130_IL-6R cells were stimulated with human IL-6 and soluble IL-6R to allow IL-6 classical and trans signalling-induced proliferation. PE containing anti-Mesothelin Fab reduces proliferation of Ba/F3_gp130_IL-6R cells to 85%, suggesting that PE alone slightly blocks proliferation of these cells. About half of the 27 Fabs tested (for example, clone 01, 06, 16, and 26) inhibit the proliferation of Ba/F3_gp130_IL-6R cells more than the anti-Mesothelin Fab suggesting blockade of IL-6 classical signalling-induced proliferation by these Fabs (Table 2). In comparison, all 27 candidate Fabs inhibit proliferation induced by IL-6 trans signalling. In summary, these data suggest that some of the 27 candidate Fabs selectively inhibit IL-6 trans signalling, while others inhibit both IL-6 classical and trans signalling.

TABLE 2
Inhibition of combined IL-6 classical and trans signalling-induced
proliferation by PE containing each of the 27 candidate Fabs.
Proliferation of Ba/F3_gp130_IL-6R cells induced by 10 ng/ml IL-6
and 200 ng/ml soluble IL-6R (sIL-6R) was set to 100%. Results are
given as mean from n = 3 independent experiments ± SD.
Ba/F3_gp130_IL-6R

Proliferation in %

| | | | |
|---|---|---|---|
| Sample | IL-6:sIL-6R | Mean | SD |

| Ctr. | | | − | 0.0 | 0.0 |
|---|---|---|---|---|---|
| IL-6:sIL-6R | | | + | 100.0 | 0.0 |
| | Periplasmic | 1H7 | + | 84.6 | 4.5 |
| | extracts | 01 | + | 53.4 | 11.0 |
| | containing | 02 | + | 75.7 | 11.9 |
| | Fabs | 03 | + | 65.8 | 8.6 |
| | | 04 | + | 68.1 | 4.9 |
| | | 05 | + | 74.0 | 9.8 |
| | | 06 | + | 55.3 | 9.8 |
| | | 07 | + | 86.1 | 11.2 |
| | | 08 | + | 89.5 | 12.6 |
| | | 09 | + | 91.6 | 6.6 |
| | | 10 | + | 67.7 | 4.9 |
| | | 11 | + | 80.6 | 9.2 |
| | | 12 | + | 61.6 | 6.7 |
| | | 13 | + | 69.4 | 8.6 |
| | | 14 | + | 72.7 | 12.7 |
| | | 15 | + | 72.0 | 12.5 |
| | | 16 | + | 54.2 | 7.7 |
| | | 17 | + | 85.2 | 7.1 |
| | | 18 | + | 68.8 | 11.0 |
| | | 19 | + | 73.3 | 9.2 |
| | | 20 | + | 81.9 | 7.4 |
| | | 21 | + | 61.0 | 6.7 |
| | | 22 | + | 53.6 | 7.3 |
| | | 23 | + | 84.5 | 6.8 |
| | | 24 | + | 73.0 | 15.6 |
| | | 25 | + | 69.7 | 12.6 |
| | | 26 | + | 41.0 | 8.8 |
| | | 27 | + | 62.3 | 11.4 |

To quantify the selectivity of each Fab for inhibiting IL-6 trans signalling, the ratio between normalized, residual cytokine-induced proliferation of Ba/F3_gp130_IL-6R cells (measuring IL-6 classical signaling) (Table 2) and Ba/F3_gp130 cells (measuring IL-6 trans signalling) (Table 1) in the presence of each candidate Fab was calculated. This ratio is called classical:trans signalling ratio. Fabs that inhibit IL-6 trans signalling to a greater degree than IL-6 classical signalling (i.e. greater or stronger inhibition of IL-6 trans signalling) are characterised by a higher classical:trans signalling ratio (e.g. a ratio of >1.0). Fabs that inhibit IL-6 trans and classical signalling to the same extent have a classical:trans signalling ratio of approximately 1.0, while Fabs that inhibit IL-6 classical signalling to a greater degree than IL-6 trans signalling (i.e. greater or stronger inhibition of IL-6 classical signalling) are characterised by a low classical:trans signalling ratio (i.e. a ratio of <1.0). Accordingly, Fabs were sorted in descending order of classical:trans signalling ratio (Table 3). In particular, Fabs 20, 07, 19, 09, and 12 have a high classical:trans signalling ratio (2.2, 1.5, 1.4, 1.3 and 1.3 respectively) and are thus promising candidates for selective inhibition (or greater inhibition) of IL-6 trans signalling.

TABLE 3
Ranking of candidate Fabs based on inhibition of proliferation.
Mean residual cytokine-induced proliferation in the presence of the
indicated Fabs taken from Table 1 (Ba/F3_gp130, measuring IL-6 trans
signalling) and Table 2 (Ba/F3_gp130_IL-6R, measuring combined IL-6
classical and trans signalling) respectively. Fabs are sorted in
descending order of classical:trans signalling ratio.

| | | | Classical:trans |
|---|---|---|---|
| | Residual | Residual | signalling |
| | proliferation of | proliferation of | ratio |
| Fab | Ba/F3_gp130_IL-6R | Ba/F3_gp130 | proliferation |
| 20 | 82 | 37 | 2.2 |
| 07 | 86 | 56 | 1.5 |
| 19 | 73 | 53 | 1.4 |
| 09 | 92 | 70 | 1.3 |
| 12 | 62 | 48 | 1.3 |
| 27 | 62 | 51 | 1.2 |
| 01 | 53 | 44 | 1.2 |
| 25 | 70 | 58 | 1.2 |
| 14 | 73 | 61 | 1.2 |
| 26 | 41 | 35 | 1.2 |
| 18 | 69 | 60 | 1.2 |
| 17 | 85 | 74 | 1.2 |
| 16 | 54 | 49 | 1.1 |
| 05 | 74 | 67 | 1.1 |
| 10 | 68 | 62 | 1.1 |
| 02 | 76 | 69 | 1.1 |
| 15 | 72 | 67 | 1.1 |
| 03 | 66 | 61 | 1.1 |
| 11 | 81 | 76 | 1.1 |
| 21 | 61 | 59 | 1.0 |
| 04 | 68 | 66 | 1.0 |
| 13 | 69 | 67 | 1.0 |
| 23 | 85 | 84 | 1.0 |
| 08 | 90 | 90 | 1.0 |
| 24 | 73 | 76 | 1.0 |
| 06 | 55 | 59 | 0.9 |
| 22 | 54 | 58 | 0.9 |
| 1H7 | 83 | 101 | 0.8 |

### Selective Inhibition of IL-6 Trans Signalling-Induced Intracellular Signalling by Fabs

Next, the capacity of the 27 candidate Fabs to selectively inhibit intracellular IL-6 trans signalling was tested. In contrast to cell proliferation, which takes hours to days, intracellular signalling occurs within seconds to minutes. Therefore, signalling is unaffected by indirect events and serves as a direct measure for IL-6 signalling. Both IL-6 classical and trans signalling lead to the activation of gp130-associated Januskinases (JAK). Activated JAKs phosphorylate tyrosine (Y) residues within the intracellular part of g130. These phosphorylated Y motifs recruit signal transducers and activators of transcription 3 (STAT3). STAT3 is then phosphorylated at Y705 by JAKs (reviewed in Schaper and Rose-John. Cytokine Growth Factor Rev. 015 October; 26(5):475-487). Hence, phosphorylation of STAT3 at Y705 serves as a readout for IL-6-induced intracellular signalling.

To quantify the selectivity of each Fab for inhibiting IL-6 trans signalling, the ratio between residual STAT3 activation (i.e. phosphorylation on Y705) induced by stimulation with IL-6 and soluble IL-6R in HEK293_IL-6R cells (measuring combined IL-6 classical and trans (mixed) signalling) and HEK293 cells (measuring IL-6 trans signalling) in the presence of individual candidate Fabs was calculated (classical:trans signalling ratio).

As described above, clones that selectively inhibit IL-6 trans signalling (i.e. a greater inhibition of IL-6 trans signalling compared to IL-6 classical signalling) are characterized by a higher classical:trans signalling ratio (e.g. >1.0). Accordingly, clones were sorted in descending order of classical:trans signalling ratio (Table 4). Remarkably, the same five Fabs (clones 20, 19, 07, 09, and 12) were ranked among the Fabs with the highest classical:trans signalling ratio for intracellular signalling.

TABLE 4
Ranking of candidate Fabs based on inhibition
of intracellular signalling. Mean residual cytokine-
induced STAT3 Y705 phosphorylation in the presence of
the indicated Fabs in HEK293_IL-6R cells (measuring
combined IL-6 classical and trans (mixed) signalling)
and HEK293 cells (measuring IL-6 trans signalling).
Fabs are sorted in descending order of
classical:trans signalling ratio.

| | | | Classical:trans |
|---|---|---|---|
| | Residual STAT3 | Residual STAT3 | signalling |
| | phosphorylation in | phosphorylation in | ratio intracellular |
| Fab | HEK293_IL-6R | HEK293 | signalling |
| 20 | 97 | 32 | 3.0 |
| 19 | 72 | 24 | 3.0 |
| 07 | 77 | 28 | 2.8 |
| 26 | 94 | 39 | 2.4 |
| 09 | 83 | 38 | 2.2 |
| 12 | 90 | 42 | 2.1 |
| 25 | 88 | 42 | 2.1 |
| 18 | 89 | 46 | 1.9 |
| 17 | 71 | 38 | 1.9 |
| 10 | 100 | 54 | 1.8 |
| 21 | 80 | 45 | 1.8 |
| 22 | 72 | 42 | 1.7 |
| 16 | 80 | 46 | 1.7 |
| 27 | 82 | 51 | 1.6 |
| 08 | 93 | 58 | 1.6 |
| 14 | 86 | 55 | 1.6 |
| 01 | 85 | 55 | 1.5 |
| 15 | 72 | 48 | 1.5 |
| 04 | 79 | 54 | 1.5 |
| 13 | 80 | 55 | 1.5 |
| 06 | 80 | 57 | 1.4 |
| 11 | 85 | 60 | 1.4 |
| 05 | 75 | 54 | 1.4 |
| 24 | 70 | 54 | 1.3 |
| 02 | 74 | 59 | 1.3 |
| 23 | 68 | 57 | 1.2 |
| 03 | 63 | 54 | 1.2 |

### Example 2—Production and Functional Characterisation of Anti-IL-6R IgG Antibodies

### Materials and Methods

### Production of Anti-IL-6R IgG Antibodies by Chimerization of Selected Fab Fragments to Human IgG1

Expression vectors for Fabs chimerized with human IgG1 were transfected to CHO cells. Cell culture supernatants were collected and anti-IL-6R IgG antibody concentrations was determined.

ELISA assay to analyse binding of anti-IL-6R IgG antibodies to soluble IL-6R

To enable proper folding of soluble IL-6R, ELISA plates were first coated with neutravidin (500 ng/well), followed by biotinylated IL-6 (500 ng/ml) and subsequently incubated with recombinant human soluble IL-6R (500 ng/ml). The plates were then incubated with increasing concentrations of the anti-IL-6R IgG antibodies (1 ng/ml, 10 ng/ml, 20 ng/ml, 50 ng/ml, 100 ng/ml and 500 ng/ml) and subsequently detected by incubating with HRP-coupled anti-human Fc secondary antibodies. Anti-Mesothelin IgG antibody (clone 1H7) was used as a negative control.

### Flow Cytometry Assay to Analyse Binding of Anti-IL-6R IgG Antibodies to Membrane-Bound IL-6R

Human THP1 cells (human monocytic cell line derived from a leukemia patient, commercially available) expressing membrane-bound IL-6R were incubated with anti-IL-6R IgG antibodies. After washing, cells were incubated with APC-coupled secondary antibodies and fluorescence quantified using flow cytometry. Cells stained only with APC-coupled secondary antibodies were used as control for unspecific binding of secondary antibodies. Binding of anti-IL-6R IgG antibodies to membrane-bound IL-6R is indicated by an increase in mean fluorescence of cells stained with anti-IL-6R IgG antibodies and secondary antibodies compared to cells stained with secondary antibodies only.

To analyse antibody binding to membrane-bound IL-6R in the presence of IL-6, Ba/F3_gp130_IL-6R cells were washed twice with 1% BSA-EDTA buffer (1% BSA, 0.5 mM EDTA in PBS). Cells were then incubated on ice with the anti-IL-6R IgG C20 (1:100, 440 ng/ml in 1% BSA-EDTA buffer) in the absence or presence of an equimolar amount of human IL-6 (Peprotech, 60 ng/ml) for 30 min. Subsequently, cells were stained with specific anti-human secondary antibodies coupled to PE (1:100, Biozol, in 1% BSA-EDTA buffer) for 30 min. Ba/F3_gp130_IL-6R cells incubated with secondary antibody only served as a control for unspecific binding of secondary antibodies. Analysis was performed by flow cytometry using a FACS Canto II (BD Biosciences), binding of anti-IL-6R IgG C20 to membrane-bound IL-6R in the absence or presence of IL-6 is indicated by an increase in mean fluorescence of cells stained with anti-IL-6R IgG C20 and secondary antibodies compared to cells stained with secondary antibodies only.

### Cell Proliferation Assay to Analyse Inhibition of Human IL-6, Human IL-11, Murine IL-6 and Human IL-3 Signalling Pathways by Anti-IL-6R IgG Antibodies

Two variants of Ba/F3 cells were used as a cellular model for IL-6-mediated cell proliferation as described in Example 1.

To analyse cytokine-induced cell proliferation mediated by the human IL-6 trans, IL-6 classical, or combined IL-6 classical and trans (IL-6 mixed) pathways, 5,000 cells/well were seeded in 96-well plates in DMEM (10% FCS+penicillin/streptomycin). Ba/F3_gp130_IL-6R cells were cultured in the presence of human IL-6 (Peprotech, 10 ng/ml) to stimulate IL-6 classical signalling, or cultured in the presence of IL-6 (10 ng/ml) and soluble IL-6R (200 ng/ml) to stimulate combined IL-6 classical and trans (mixed) signalling. Ba/F3_gp130 cells were cultured in the presence of IL-6 (10 ng/ml) and soluble IL-6R (200 ng/ml) to stimulate IL-6 trans signalling. Aliquots of the cells were additionally incubated with increasing concentrations of anti-IL-6R IgG antibodies (10 ng/ml, 100 ng/ml, 300 ng/ml, 500 ng/ml, 700 ng/ml, 1,000 ng/ml and 10,000 ng/ml for Ba/F3_gp130 cells, or 1 μg/ml and 10 μg/ml for Ba/F3_gp130_IL-6R cells), or an anti-Mesothelin IgG antibody (1H7) (1 μg/ml) which was used as a control for unspecific inhibition of intracellular signalling by IgG antibodies. After 48 h the number of viable cells was determined as described in Example 1.

To analyse cell proliferation induced by Hyper-IL-6 (Hy-IL-6) trans signalling, Ba/F3_gp130 cells were seeded as described above and Hy-IL-6 (28 ng/ml, equivalent molarity to 10 ng/ml IL-6) was added to stimulate IL-6 trans-signalling. Aliquots of the cells were additionally incubated with increasing concentrations of anti-IL-6R IgG antibodies (10 ng/ml, 100 ng/ml, 300 ng/ml, 500 ng/ml, 700 ng/ml, 1,000 ng/ml and 10,000 ng/ml, or an anti-Mesothelin IgG antibody (1H7) (1 μg/ml) which was used as a control for unspecific inhibition of intracellular signalling by IgG antibodies. After 48 h the number of viable cells was determined as described in Example 1.

To analyse cytokine-induced cell proliferation mediated by the human IL-11 trans, IL-11 classical, or combined IL-11 classical and trans (IL-11 mixed) pathways, cells were seeded, stimulated and analysed as described above, however Ba/F3_gp130_IL-6R cells were replaced with Ba/F3_gp130_IL-11R cells (expressing human gp130 and human IL-11R), IL-6 and soluble IL-6R were replaced with IL-11 (Bio-techne, 10 ng/ml) and soluble IL-11R (Bio-techne, 200 ng/ml) respectively, and the anti-IL-6R IgG antibodies were added at a concentration of 10 μg/ml.

To analyse cytokine-induced cell proliferation mediated by the murine IL-6 trans, IL-6 classical, or combined IL-6 classical and trans (IL-6 mixed) pathways, cells were seeded, stimulated and analysed as described above, however Ba/F3_gp130_IL-6R cells were replaced with Ba/F3_gp130_muIL-6R cells (expressing human gp130 and murine IL-6R), IL-6 and soluble IL-6R were replaced with murine IL-6 (Bio-techne, 10 ng/ml) and murine soluble IL-6R (Bio-techne, 200 ng/ml) respectively, and the anti-IL-6R IgG antibodies were added at a concentration of 10 μg/ml.

To analyse cytokine-induced proliferation mediated by IL-3 signalling, Ba/F3 wildtype cells were used. Ba/F3 wildtype cells do not express membrane-bound gp130 or IL-6R and are thus not responsive to IL-6, however Ba/F3 wildtype cells proliferate in response to stimulation with IL-3. Ba/F3 wildtype cells were thus seeded as described above and stimulated with recombinant murine IL-3 (10 ng/ml, Peprotech 213-13-2) and incubated with 1 or 10 μg/ml of anti-IL-6R IgG antibodies. After 48 h the number of viable cells was determined as described in Example 1.

Intracellular signalling assay to analyse inhibition of human IL-6 signalling pathways by anti-IL-6R IgG antibodies Two variants of human embryonic kidney cells (HEK293) were used as a cellular model for intracellular signalling as described in Example 1.

To analyse cytokine-induced intracellular signalling mediated by the IL-6 trans, IL-6 classical, or combined IL-6 classical and trans (IL-6 mixed) pathways, IL-6-induced STAT3 Y705 phosphorylation was determined by intracellular flow cytometry. HEK293 and HEK293_IL-6R cells were seeded and cultured as described in Example 1. HEK293_IL-6R cells were treated for 30 mins with IL-6 (10 ng/ml) to stimulate IL-6 classical signalling, or treated for 30 mins with IL-6 (10 ng/ml) and soluble IL-6R (200 ng/ml) to stimulate combined IL-6 classical and trans (mixed) signalling. HEK293 cells were treated for 30 mins with IL-6 (10 ng/ml) and soluble IL-6R (200 ng/ml) to stimulate IL-6 trans signalling. Aliquots of the cells were additionally incubated with anti-IL-6R IgG antibodies (1 μg/ml) or an anti-Mesothelin IgG antibody (1H7) (1 μg/ml) which was used as a control for unspecific inhibition of intracellular signalling by IgG antibodies. Cells were fixed, stained and analysed by flow cytometry as described in Example 1.

### Results

### Generation of Five Anti-IL-6R IgG Antibodies by Chimerization of Fab Fragments to Human IgG1

The initial Fab screening highlights five clones, namely clones 07, 09, 12, 19, and 20 as potential selective inhibitors of IL-6 trans signalling. To further characterize their ability to inhibit IL-6 trans signalling these five Fabs were chosen for chimerization with human IgG1.

### Anti-IL-6R IgG Antibodies Bind to Human Soluble IL-6R and Human Membrane-Bound IL-6R

First, the ability of the five anti-IL-6R IgG antibodies to bind soluble IL-6R was quantified by ELISA. All five anti-IL-6R IgGs dose-dependently bind soluble IL-6R, while no binding was detected for the anti-Mesothelin IgG (1H7) control antibody (FIG. 1).

Second, the binding capacity of the five anti-IL-6R IgG antibodies to membrane-bound IL-6R expressed on human THP1 cells was quantified by flow cytometry. Interestingly, all five anti-IL-6R IgG antibodies bind strongly to membrane-bound IL-6R, as shown by an increase in mean fluorescence of cells stained with anti-IL-6R IgG antibodies and secondary antibodies compared to cells stained with secondary antibodies only (FIG. 2).

This suggests, that inhibition of IL-6 trans signalling is not through specific binding of anti-IL-6R IgG antibodies to soluble IL-6R, but rather through interference with the formation of the active IL-6R complex.

Furthermore, the ability of anti-IL-6R IgG C20 to bind membrane-bound IL-6R expressed on Ba/F3_gp130_IL-6R (expressing human IL-6R) in the absence and presence of IL-6 was quantified by flow cytometry. Anti-IL-6R IgG C20 binds membrane-bound IL-6R both in the absence and presence of IL-6, as shown by an increase in mean fluorescence of cells stained with anti-IL-6R IgG C20 and a secondary antibody, compared to cells stained with secondary antibodies only (FIG. 3). Thus, the presence or absence of IL-6 does not affect binding of anti-IL-6R IgG C20 to membrane-bound IL-6R.

### Selective Inhibition of IL-6 Trans Signalling-Induced Cell Proliferation by Anti-IL-6R IgG Antibodies

In the next step, the ability of the five anti-IL-6R IgG antibodies to selectively block IL-6 trans signalling-induced cell proliferation was analyzed by using the Ba/F3 cellular system.

As expected, Ba/F3_gp130 cells do not proliferate in response to stimulation with IL-6 or soluble IL-6R alone. Stimulation with both IL-6 and soluble IL-6R results in IL-6 trans signalling-induced proliferation. The anti-Mesothelin IgG (1H7) control antibody does not affect IL-6 trans signalling-induced proliferation of Ba/F3_gp130 cells, suggesting that proliferation is not affected by IgG antibodies in an unspecific manner. All five anti-IL-6R IgG antibodies reduce IL-6 trans signalling-induced proliferation dose dependently, with IgG 09 being the least potent inhibitor (Table 5 and FIG. 4).

TABLE 5
Inhibition of IL-6 trans signalling-induced proliferation
by anti-IL-6R IgG antibodies. Proliferation of Ba/F3_gp130 cells
induced by 10 ng/ml IL-6 and 200 ng/ml sIL-6R was set to 100%.
Results are given as mean from n = 3 independent experiments ± SD.
Ba/F3_gp130

Proliferation in %

| Sample | Cytokine | IgG [ng/ml] | Mean | SD |
|---|---|---|---|---|
| Ctr | — | | 0.0 | 0.0 |
| IL-6 | IL-6 | | 0.9 | 1.8 |
| sIL-6R | sIL-6R | | 1.8 | 1.8 |
| IL-6 + | IL-6 + | | 100.0 | 0.0 |
| sIL-6R | sIL-6R | | | |
| 1 H7 | IL-6 + | 1,000 | 100.0 | 7.3 |
| | sIL-6R | | | |
| IgG 07 | IL-6 + | 10 | 98.8 | 8.6 |
| | sIL-6R | 100 | 92.2 | 4.7 |
| | | 300 | 78.3 | 8.6 |
| | | 500 | 70.2 | 5.4 |
| | | 700 | 55.3 | 8.7 |
| | | 1,000 | 41.2 | 4.9 |
| | | 10,000 | 9.6 | 2.8 |
| IgG 09 | IL-6 + | 10 | 98.2 | 3.0 |
| | sIL-6R | 100 | 93.1 | 2.4 |
| | | 300 | 88.3 | 1.1 |
| | | 500 | 82.6 | 6.8 |
| | | 700 | 82.5 | 4.3 |
| | | 1,000 | 72.9 | 1.6 |
| | | 10,000 | 25.8 | 2.1 |
| IgG 12 | IL-6 + | 10 | 101.2 | 2.4 |
| | sIL-6R | 100 | 98.9 | 3.0 |
| | | 300 | 89.1 | 2.6 |
| | | 500 | 75.3 | 2.6 |
| | | 700 | 66.9 | 4.4 |
| | | 1,000 | 48.1 | 2.4 |
| | | 10,000 | 11.6 | 2.8 |
| IgG 19 | IL-6 + | 10 | 99.2 | 5.2 |
| | sIL-6R | 100 | 92.2 | 2.3 |
| | | 300 | 88.5 | 1.0 |
| | | 500 | 78.3 | 5.1 |
| | | 700 | 72.3 | 3.4 |
| | | 1,000 | 57.7 | 5.7 |
| | | 10,000 | 10.8 | 6.6 |
| IgG 20 | IL-6 + | 10 | 102.7 | 8.9 |
| | sIL-6R | 100 | 88.5 | 24.7 |
| | | 300 | 84.8 | 16.2 |
| | | 500 | 52.8 | 15.4 |
| | | 700 | 36.1 | 19.2 |
| | | 1,000 | 16.3 | 4.1 |
| | | 10,000 | 6.5 | 5.6 |

Using the mean IgG 07, 09, 12 and 19 values for % IL-6 trans signalling-induced proliferation in Table 5, and an updated mean value for IgG 20 which includes the data used to calculate the mean values in Table 5 in addition to one further experimental repeat (Table 6), dose dependency curves for the inhibition of IL-6 trans signalling-induced cell proliferation by the anti-IL-6R IgG antibodies were plotted (FIG. 5) and IC50 values calculated (Table 7). Low nM IC50 values for the anti-IL-6R IgG antibodies indicate a potent inhibition of IL-6 trans signalling by the antibodies.

TABLE 6
Inhibition of IL-6 trans signalling-induced proliferation
by anti-IL-6R IgG C20. Proliferation of Ba/F3_gp130 cells induced
by 10 ng/ml IL-6 and 200 ng/ml sIL-6R was set to 100%. Results
are given as mean from n = 4 independent experiments ± SD.
Ba/F3_gp130

Proliferation in %

| Sample | Cytokine | IgG [ng/ml] | Mean | SD |
|---|---|---|---|---|
| IgG 20 | IL-6 + sIL- | 10 | 106.8 | 5.3 |
| | 6R | 100 | 104.0 | 4.2 |
| | | 300 | 88.8 | 10.5 |
| | | 500 | 57.8 | 7.7 |
| | | 700 | 43.6 | 15.5 |
| | | 1,000 | 19.5 | 2.9 |
| | | 10,000 | 7.6 | 7.5 |

TABLE 7
IC50 of inhibition of IL-6 trans signalling-induced proliferation
by anti-IL-6R IgG antibodies. IC50 values, in ng/ml and nM, for the
inhibition of proliferation of Ba/F3_gp130 cells induced by 10 ng/ml
IL-6 and 200 ng/ml sIL-6R by increasing concentrations of the five
anti-IL-6R IgG antibodies (10 ng/ml, 100 ng/ml, 300 ng/ml, 500 ng/ml,
700 ng/ml, 1,000 ng/ml and 10,000 ng/ml).
Ba/F3_gp130

IC50

| IgG | Cytokine | ng/ml | nM |
|---|---|---|---|
| 07 | IL-6 + sIL-6R | 757 | 5 |
| 09 | IL-6 + sIL-6R | 5260 | 35 |
| 12 | IL-6 + sIL-6R | 863 | 5.8 |
| 19 | IL-6 + sIL-6R | 1214 | 8 |
| 20 | IL-6 + sIL-6R | 531 | 3.5 |

To provide further evidence for the inhibition of IL-6 trans signalling by the five anti-IL-6R IgG antibodies, Hyper-IL-6 (Hy-IL-6) was used to induce IL-6 trans signalling. Hy-L-6 is a recombinant fusion protein consisting of IL-6 and soluble IL-6R (Peters et al., J Immunol., 1998; 161(7): 3575-81) and is a tool frequently used to induce IL-6 trans signalling in situations where theoretically IL-6 classical and trans signalling are activated in parallel, and is thus a useful tool for in vivo analysis of the biological consequences of IL-6 trans signalling. All five anti-IL-6R IgG antibodies inhibit Hy-IL-6 trans signalling-induced proliferation, with low nM IC50 values for the inhibition of Hy-IL-6-induced cell proliferation by the anti-IL-6R IgG 10 antibodies further supporting a potent inhibition of IL-6 trans signalling by the antibodies (Table 8).

TABLE 8
IC50 of inhibition of Hy-IL-6 trans signalling-induced
proliferation by anti-IL-6R IgG antibodies. IC50 values,
in ng/ml and nM, for the inhibition of proliferation of
Ba/F3_gp130 cells induced by 28 ng/ml Hy-IL-6 by increasing
concentrations of the five anti-IL-6R IgG antibodies (10 ng/ml,
100 ng/ml, 300 ng/ml, 500 ng/ml, 700 ng/ml, 1,000 ng/ml
and 10,000 ng/ml).
Ba/F3_gp130

IC50

| IgG | Cytokine | ng/ml | nM |
|---|---|---|---|
| 07 | Hy-IL-6 | 644 | 4.3 |
| 09 | Hy-IL-6 | 4130 | 28 |
| 12 | Hy-IL-6 | 748 | 5 |
| 19 | Hy-IL-6 | 755 | 5 |
| 20 | Hy-IL-6 | 304 | 2 |

Next, it was tested whether the anti-IL-6R IgG antibodies reduce IL-6 classical signalling-induced proliferation in Ba/F3_gp130_IL-6R cells. As before, the anti-Mesothelin IgG (1H7) control antibody has no effect on proliferation of Ba/F3_gp130_IL-6R cells, so that unspecific inhibition of proliferation by IgG antibodies can be excluded. It is noteworthy that none of the anti-IL-6R IgG antibodies investigated significantly inhibit proliferation induced by IL-6 classical signalling (Table 9 and FIG. 6), even at a concentration of 10 μg/ml which causes a large inhibition of IL-6 trans signalling-induced proliferation (Table 5). As there is less than the 50% inhibition required to calculate IC50 values at the maximum concentration of 10 μg/ml (53 nM), all five anti-IL-6R IgG antibody will have an IC50 for the inhibition of IL-6 classical signalling-induced proliferation of greater than 10 μg/ml (53 nM). Thus, all five anti-IL-6R IgG antibodies investigated selectively block IL-6 trans signalling-induced proliferation without affecting IL-6 classical signalling-induced proliferation (i.e. inhibit IL-6 trans signalling while maintaining IL-6 classical signalling).

TABLE 9
Inhibition of IL-6 classical signalling-induced
proliferation by anti-IL-6R IgG antibodies. Proliferation
of Ba/F3_gp130_IL-6R cells induced by 10 ng/ml IL-6 was
set to 100%. Results are given as mean from n = 3
independent experiments ± SD.
Ba/F3_gp130_IL-6R

Proliferation in %

| Sample | IL-6 | IgG [μg/ml] | Mean | SD |
|---|---|---|---|---|
| Ctr. | − | | 0.0 | 0.0 |
| IL-6 | + | | 100.0 | 0.0 |
| 1H7 | + | 10 | 96.4 | 1.8 |
| IgG 07 | + | 1 | 92.0 | 0.5 |
| | | 10 | 90.8 | 1.2 |
| IgG 09 | + | 1 | 100.3 | 1.8 |
| | | 10 | 94.0 | 1.7 |
| IgG 12 | + | 1 | 94.3 | 4.4 |
| | | 10 | 92.2 | 2.8 |
| IgG 19 | + | 1 | 100.1 | 0.4 |
| | | 10 | 93.1 | 2.4 |
| IgG 20 | + | 1 | 92.6 | 2.5 |
| | | 10 | 87.56 | 5.2 |

As described in Example 1, antibodies that are believed to selectively inhibit IL-6 trans signalling (i.e. a greater inhibition of IL-6 trans signaling compared to IL-6 classical signalling) are characterized by a higher classical:trans signalling ratio (e.g. >1.0) and a greater ratio indicates a greater selectivity of inhibition of IL-6 trans signalling compared to IL-6 classical signalling. Accordingly, the classical:trans signalling ratio was calculated for all five anti-IL-6R IgG antibodies (Table 10).

TABLE 10
Cell proliferation classical:trans signalling ratio
of anti-IL-6R IgG antibodies. Mean residual cytokine-
induced proliferation in the presence of the indicated
IgG antibodies taken from Table 5 (Ba/F3_gp130, measuring
IL-6 trans signalling) and Table 9 (Ba/F3_gp130_IL-6R,
measuring IL-6 classical signalling) respectively.

| | | Residual | Residual | Classical:trans |
|---|---|---|---|---|
| | | proliferation of | proliferation of | signalling ratio |
| IgG | IgG [μg/ml] | Ba/F3_gp130_IL-6R | Ba/F3_gp130 | proliferation |
| 07 | 1 | 92 | 41 | 2.2 |
| | 10 | 91 | 10 | 9.1 |
| 09 | 1 | 100 | 73 | 1.4 |
| | 10 | 94 | 26 | 3.6 |
| 12 | 1 | 94 | 48 | 2.0 |
| | 10 | 92 | 12 | 7.7 |
| 19 | 1 | 100 | 58 | 1.7 |
| | 10 | 93 | 11 | 8.5 |
| 20 | 1 | 93 | 16 | 5.8 |
| | 10 | 88 | 7 | 12.6 |

To analyse the ability of the five anti-IL-6R IgG antibodies to inhibit IL-6-induced proliferation under more physiological conditions, Ba/F3_gp130_IL-6R cells were stimulated with a mixture of IL-6 and soluble IL-6R. As described in Example 1, this condition allows both IL-6 classical and trans (mixed) signalling to occur. None of the anti-IL-6R IgG antibodies investigated significantly inhibit proliferation induced by combined IL-6 classical and trans (mixed) signalling (Table 11 and FIG. 7). As there is less than the 50% inhibition required to calculate IC50 values at the maximum concentration of 10 μg/ml (53 nM), all five anti-IL-6R IgG antibody will have an IC50 for the inhibition of IL-6 mixed signalling-induced proliferation of greater than 10 μg/ml (53 nM). Thus, blockade of IL-6 trans signalling with the anti-IL-6R IgG antibodies does not prevent IL-6 classical signalling under the analysed conditions.

TABLE 11
Inhibition of combined IL-6 classical and trans
signalling-induced proliferation by anti-IL-6R IgG
antibodies. Proliferation of Ba/F3_gp130_IL-6R cells
induced by 10 ng/ml IL-6 and 200 ng/ml sIL-6R was set
to 100%. Results are given as mean from n = 3
independent experiments ± SD.
Ba/F3_gp130_IL-6R

| | | | | Proliferation in % | |
|---|---|---|---|---|---|
| Sample | IL-6 | sIL-6R | IgG [μg/ml] | Mean | SD |
| Ctr. | − | − | | 0.0 | 0.0 |
| IL-6 | + | − | | 100.0 | 0.0 |
| sIL-6R | − | + | | 7.61 | 4.18 |
| IL-6 + sIL-6R | + | + | | 94.4 | 0.9 |
| IgG 1H7 | + | + | 10 | 97.8 | 1.0 |
| IgG 07 | + | + | 1 | 93.0 | 0.7 |
| | | | 10 | 85.9 | 4.2 |
| IgG 09 | + | + | 1 | 100.9 | 4.7 |
| | | | 10 | 97.6 | 5.4 |
| IgG 12 | + | + | 1 | 94.3 | 2.8 |
| | | | 10 | 87.6 | 5.4 |
| IgG 19 | + | + | 1 | 104.4 | 1.7 |
| | | | 10 | 95.2 | 5.6 |
| IgG 20 | + | + | 1 | 94.7 | 1.2 |
| | | | 10 | 88.6 | 5.4 |

As described in Example 1, combined IL-6 classical and trans (mixed) signalling represents IL-6 signalling under physiological conditions. The IL-6 signalling ratio under physiological conditions (mixed:trans signalling ratio) was also calculated for all five anti-IL-6R IgG antibodies (Table 12).

TABLE 12
Cell proliferation mixed:trans signalling ratio of anti-IL-6R
IgG antibodies. Mean residual cytokine-induced proliferation
in the presence of the indicated IgG antibodies taken from Table 5
(Ba/F3_gp130, measuring IL-6 trans signalling) and Table 11
(Ba/F3_gp130_IL-6R, measuring combined IL-6 classical
and trans (mixed) signalling) respectively.

| | | Residual | Residual | |
|---|---|---|---|---|
| | | proliferation | proliferation | Mixed:trans |
| | | of Ba/ | of Ba/ | signalling ratio |
| IgG | IgG [μg/ml] | F3_gp130_IL-6R | F3_gp130 | proliferation |
| 07 | 1 | 93 | 41 | 2.3 |
| | 10 | 86 | 10 | 8.6 |
| 09 | 1 | 101 | 73 | 1.4 |
| | 10 | 98 | 26 | 3.8 |
| 12 | 1 | 94 | 48 | 2.0 |
| | 10 | 88 | 12 | 7.3 |
| 19 | 1 | 104 | 58 | 1.8 |
| | 10 | 95 | 11 | 8.6 |
| 20 | 1 | 95 | 16 | 5.9 |
| | 10 | 89 | 7 | 12.7 |

The classical:trans signalling IC50 ratio was also calculated for the five anti-IL-6R IgG antibodies, using the IC50 values for the inhibition of IL-6 trans signalling-induced proliferation in Table 7, and using the minimum IC50 value of 10 μg/ml, or 53 nM, for the inhibition of IL-6 classical signalling-induced proliferation.

TABLE 13
Cell proliferation classical:trans signalling IC50 ratio of anti-IL-6R
IgG antibodies. IC50 values for the inhibition of IL-6 trans signalling-
induced proliferation by the anti-IL-6R IgG antibodies (Table 7) and
the minimum IC50 value for inhibition of IL-6 classical-signalling
induced proliferation by the anti-IL-6R IgG antibodies.

| | IC50 for inhibition | IC50 for inhibition | |
|---|---|---|---|
| | of IL-6 trans | of IL-6 classical | Classical:trans IC50 |
| | signalling-induced | signalling-induced | signalling ratio |
| IgG | proliferation (nM) | proliferation (nM) | proliferation |
| 07 | 5 | >53 nM | >10 |
| 09 | 35 | >53 nM | >1.5 |
| 12 | 5.8 | >53 nM | >9 |
| 19 | 8 | >53 nM | >6 |
| 20 | 3.5 | >53 nM | >15 |

Next, to test whether the five anti-IL-6R IgG antibodies unspecifically affect proliferation, Ba/F3 wildtype cells were stimulated with IL-3. These cells proliferate in response to stimulation with IL-3 and thus proliferation of these cells should not be affected by the anti-IL-6R IgG antibodies. Regardless of the concentration studied, the five anti-IL-6R IgG antibodies do not reduce IL-6-independent proliferation (Table 14).

TABLE 14
Inhibition of IL-3-induced proliferation
by anti-IL-6R IgG antibodies. Proliferation of Ba/F3
wildtype cells induced by 10 ng/ml IL-3 was set to 100%.
Results are given as mean from n = 3
independent experiments ± SD.
Ba/F3 wildtype

Proliferation in %

| Sample | IL-3 | IgG [μg/ml] | Mean | SD |
|---|---|---|---|---|
| Ctr. | − | | 0.0 | 0.0 |
| IL-3 | + | | 100.0 | 0.0 |
| IgG 07 | + | 1 | 104.4 | 11.6 |
| | | 10 | 106.4 | 8.6 |
| IgG 09 | + | 1 | 104.8 | 8.1 |
| | | 10 | 98.3 | 11.9 |
| IgG 12 | + | 1 | 101.9 | 9.6 |
| | | 10 | 106.5 | 7.5 |
| IgG 19 | + | 1 | 103.5 | 5.9 |
| | | 10 | 105.1 | 8.4 |
| IgG 20 | + | 1 | 103.8 | 13.1 |
| | | 10 | 108.0 | 11.5 |

Subsequently, to test whether the five anti-IL-6R IgG antibodies affect signalling via other human IL-6 family member cytokines, the effect of the five anti-IL-6R IgG antibodies on human IL-11 classical, trans, and combined classical and trans (mixed) signalling was analysed.

To analyse IL-11 classical signalling, Ba/F3_gp13_IL-R cells, expressing human gp130 and human IL-11R, were stimulated with IL-11. The anti-Mesothelin IgG (11H7) control antibody has no effect on proliferation of Ba/F3_gp130_IL-11R cells, so that unspecific inhibition of proliferation by IgG antibodies can be excluded. None of the anti-IL-6R IgG antibodies investigated significantly inhibit proliferation induced by IL-11 classical signalling even at a concentration of 10 μg/ml (Table 15).

TABLE 15
Inhibition of IL-11 classical signalling-induced
proliferation by anti-IL-6R IgG antibodies.
Proliferation of Ba/F3_gp130_IL-11R cells induced
by 10 ng/ml IL-11 was set to 100%.
Results are given as mean from n = 3
independent experiments ± SD.
Ba/F3_gp130_IL-11R

Proliferation in %

| Sample | Cytokine | IgG [ng/ml] | Mean | SD |
|---|---|---|---|---|
| Ctr. | — | — | 0.0 | 0.0 |
| IL-11 | IL-11 | — | 100.0 | 0.0 |
| 1H7 | IL-11 | 10 μg/ml | 102.4 | 1.5 |
| IgG 07 | IL-11 | 10 μg/ml | 102.5 | 3.2 |
| IgG 09 | IL-11 | 10 μg/ml | 103.6 | 0.7 |
| IgG 12 | IL-11 | 10 μg/ml | 99.5 | 5.1 |
| IgG 19 | IL-11 | 10 μg/ml | 103.7 | 2.9 |
| IgG 20 | IL-11 | 10 μg/ml | 103.8 | 6.5 |

To analyse whether the five anti-IL-6R IgG antibodies affect IL-11 trans signalling, Ba/F3_gp130 cells were stimulated with IL-11 and soluble IL-11R. None of the anti-IL-6R IgG antibodies investigated significantly inhibit proliferation induced by IL-11 trans signalling even at a concentration of 10 μg/ml (Table 16), a concentration at which a large inhibition of IL-6 trans signalling was observed (Table 5).

TABLE 16
Inhibition of IL-11 trans signalling-induced proliferation
by anti-IL-6R IgG antibodies. Proliferation of Ba/F3_gp130 cells
induced by 10 ng/ml IL-11 and 200 ng/ml soluble IL-11R (sIL-11R)
was set to 100%. Results are given as mean from
n = 3 independent experiments ± SD.
Ba/F3_gp130

| | | | Proliferation in % | |
|---|---|---|---|---|
| Sample | Cytokine | IgG [ng/ml] | Mean | SD |
| Ctr. | — | — | 0.0 | 0.0 |
| IL-11 + | IL-11 + sIL-11R | — | 100.0 | 0.0 |
| sIL-11R | | | | |
| 1H7 | IL-11 + sIL-11R | 10 μg/ml | 98.0 | 2.0 |
| IgG 07 | IL-11 + sIL-11R | 10 μg/ml | 107.1 | 3.5 |
| IgG 09 | IL-11 + sIL-11R | 10 μg/ml | 116.7 | 0.5 |
| IgG 12 | IL-11 + sIL-11R | 10 μg/ml | 111.0 | 2.6 |
| IgG 19 | IL-11 + sIL-11R | 10 μg/ml | 119.6 | 5.5 |
| IgG 20 | IL-11 + sIL-11R | 10 μg/ml | 112.1 | 5.3 |

To analyse whether the five anti-IL-6R IgG antibodies affect combined IL-11 classical and trans (mixed) signalling, Ba/F3_gp130_IL-11R cells were stimulated with IL-11 and soluble IL-11R. None of the anti-IL-6R IgG antibodies investigated significantly inhibit proliferation induced by IL-11 mixed signalling even at a concentration of 10 μg/ml (Table 17).

TABLE 17
Inhibition of IL-11 mixed signalling-induced proliferation
by anti-IL-6R IgG antibodies. Proliferation of Ba/F3_gp130_IL-11R
cells induced by 10 ng/ml IL-11 and 200 ng/ml soluble IL-11R (sIL-11R)
was set to 100%. Results are given as mean from
n = 4 independent experiments ± SD.
Ba/F3_gp130_IL-11R

| | | | Proliferation in % | |
|---|---|---|---|---|
| Sample | Cytokine | IgG [ng/ml] | Mean | SD |
| Ctr. | — | — | 0.0 | 0.0 |
| IL-11 + | IL-11 + sIL-11R | — | 100.0 | 0.0 |
| sIL-11R | | | | |
| 1H7 | IL-11 + sIL-11R | 10 μg/ml | 98.6 | 2.6 |
| IgG 07 | IL-11 + sIL-11R | 10 μg/ml | 104.6 | 7.5 |
| IgG 09 | IL-11 + sIL-11R | 10 μg/ml | 106.9 | 7.1 |
| IgG 12 | IL-11 + sIL-11R | 10 μg/ml | 105.3 | 9.3 |
| IgG 19 | IL-11 + sIL-11R | 10 μg/ml | 110.3 | 9.8 |
| IgG 20 | IL-11 + sIL-11R | 10 μg/ml | 116.0 | 9.7 |

Thus, all five anti-IL-6R IgG antibodies investigated selectively block IL-6 trans signalling-induced proliferation without affecting IL-11 signalling-induced proliferation, suggesting the anti-IL-6R IgG antibodies do not inhibit signalling via other IL-6 family cytokines (other than IL-6).

Finally, to test whether the five anti-IL-6R IgG antibodies affect murine IL-6 signalling, the effect of the five anti-IL-6R IgG antibodies on murine IL-6 classical, trans, and combined classical and trans (mixed) signalling was analysed.

To analyse murine IL-6 classical signalling, Ba/F3_gp130_muIL-6R cells, expressing human gp130 and murine IL-6R, were stimulated with murine IL-6. Notably, murine IL-6R is capable of forming an active receptor complex with human gp130. The anti-Mesothelin IgG (1-H7) control antibody has no effect on proliferation of Ba/F3_gp130_muIL-6R cells, so that unspecific inhibition of proliferation by IgG antibodies can be excluded. None of the anti-IL-6R IgG antibodies investigated significantly inhibit proliferation induced by murine IL-6 classical signalling even at a concentration of 10 μg/ml (Table 18).

TABLE 18
Inhibition of murine IL-6 classical signalling-induced proliferation
by anti- IL-6R IgG antibodies. Proliferation of Ba/F3_gp130_mulL-6R
cells induced by 10 ng/ml murine IL-6 (mulL-6) was set
to 100%. Results are given as mean from n = 3
independent experiments ± SD.
Ba/F3_gp130_muIL-6R

Proliferation in %

| Sample | Cytokine | IgG [ng/ml] | Mean | SD |
|---|---|---|---|---|
| Ctr. | — | — | 0.0 | 0.0 |
| muIL-6 | muIL-6 | — | 100.0 | 0.0 |
| 1H7 | mulL-6 | 10 μg/ml | 100.7 | 3.8 |
| IgG 07 | muIL-6 | 10 μg/ml | 100.9 | 1.0 |
| IgG 09 | mulL-6 | 10 μg/ml | 104.5 | 3.2 |
| IgG 12 | mulL-6 | 10 μg/ml | 102.3 | 2.0 |
| IgG 19 | muIL-6 | 10 μg/ml | 104.8 | 2.3 |
| IgG 20 | mulL-6 | 10 μg/ml | 107.7 | 3.8 |

To analyse whether the five anti-IL-6R IgG antibodies affect murine IL-6 trans signalling, Ba/F3gp130 cells were stimulated with murine IL-6 and murine soluble IL-6R. None of the anti-IL-6R IgG antibodies investigated significantly inhibit proliferation induced by murine IL-6 trans signalling even at a concentration of 10 μg/ml (Table 19), a concentration at which a large inhibition of human IL-6 trans signalling was observed (Table 5).

TABLE 19
Inhibition of murine IL-6 trans signalling-induced proliferation
by anti-IL-6R IgG antibodies. Proliferation of Ba/F3_gp130 cells
induced by 10 ng/ml murine IL-6 (mulL-6) and 200 ng/ml murine soluble
IL-6R (musIL-6R) was set to 100%. Results are given as
mean from n = 3 independent experiments ± SD.
Ba/F3_gp130

| | | | Proliferation in % | |
|---|---|---|---|---|
| Sample | Cytokine | IgG [ng/ml] | Mean | SD |
| Ctr. | — | — | 0.0 | 0.0 |
| mulL-6 + | mulL-6 + musIL-6R | — | 100.0 | 0.0 |
| musIL-6R | | | | |
| 1H7 | mulL-6 + musIL-6R | 10 μg/ml | 96.3 | 1.4 |
| IgG 07 | mulL-6 + musIL-6R | 10 μg/ml | 103.5 | 10.6 |
| IgG 09 | mulL-6 + musIL-6R | 10 μg/ml | 103.8 | 13.4 |
| IgG 12 | mulL-6 + musIL-6R | 10 μg/ml | 106.4 | 7.2 |
| IgG 19 | mulL-6 + musIL-6R | 10 μg/ml | 108.5 | 8.0 |
| IgG 20 | mulL-6 + musIL-6R | 10 μg/ml | 86.2 | 2.2 |

To analyse whether the five anti-IL-6R IgG antibodies affect combined murine IL-6 classical and trans (mixed) signalling, Ba/F3_gp130_muIL-6R cells were stimulated with murine IL-6 and murine soluble IL-6R. None of the anti-IL-6R IgG antibodies investigated significantly inhibit proliferation induced by murine IL-6 mixed signalling even at a concentration of 10 μg/ml (Table 20).

TABLE 20
Inhibition of murine IL-6 mixed signalling-induced proliferation
by anti-IL-6R IgG antibodies. Proliferation of Ba/F3_gp130_muIL-6R
cells induced by 10 ng/ml murine IL-6 (mulL-6) and 200 ng/ml murine
soluble IL-6R (musIL-6R) was set to 100%. Results are given as mean
from n = 3 independent experiments ± SD.
Ba/F3_gp130_muIL-6R

| | | | Proliferation in % | |
|---|---|---|---|---|
| Sample | Cytokine | IgG [ng/ml] | Mean | SD |
| Ctr. | — | — | 0.0 | 0.0 |
| mulL-6 + | mulL-6 + musIL-6R | — | 100.0 | 0.0 |
| musIL-6R | | | | |
| 1H7 | mulL-6 + musIL-6R | 10 μg/ml | 108.2 | 3.81 |
| IgG 07 | mulL-6 + musIL-6R | 10 μg/ml | 108.1 | 5.4 |
| IgG 09 | mulL-6 + musIL-6R | 10 μg/ml | 105.6 | 3.6 |
| IgG 12 | mulL-6 + musIL-6R | 10 μg/ml | 113.7 | 3.7 |
| IgG 19 | mulL-6 + musIL-6R | 10 μg/ml | 116.2 | 5.1 |
| IgG 20 | mulL-6 + musIL-6R | 10 μg/ml | 114.0 | 5.0 |

In summary, the five anti-IL-6R IgG antibodies investigated (IgG antibodies 07, 09, 12, 19 and 20) selectively inhibit human IL-6 trans signalling-induced cell proliferation without interfering with human IL-6 classical signalling-induced proliferation. In addition, the five anti-IL-6R IgG antibodies do not interfere with human IL-6-independent proliferation (e.g. IL-3), human IL-11 signalling-induced proliferation, or murine IL-6 signalling-induced proliferation.

### Selective Inhibition of IL-6 Trans Signalling-Induced Intracellular Signalling by Anti-IL-6R IgG Antibodies

Next, the ability of the anti-IL-6R IgG antibodies to interfere with IL-6 classical and trans signalling-induced JAK/STAT signalling was analysed.

First, HEK293 cells were stimulated with IL-6 and soluble IL-6R to induce IL-6 trans signalling. All five IgG antibodies strongly reduce IL-6 trans signalling-induced STAT3 phosphorylation. Of the five anti-IL-6R IgG antibodies investigated, IgG 09 is the weakest inhibitor, although it still reduces IL-6 trans signalling-induced STAT3 phosphorylation by 80% (Table 21). In contrast the anti-Mesothelin IgG (1H7) control antibody does not reduce STAT3 phosphorylation, indicating selective inhibition of IL-6 intracellular signalling by the anti-IL-6R IgG antibodies.

TABLE 21
Inhibition of IL-6 trans signalling-induced JAK/STAT signalling
by anti-IL-6R IgG antibodies. STAT3 phosphorylation of HEK293 cells
stimulated with 10 ng/ml IL-6 and 200 ng/ml soluble IL-6R (sIL-6R)
for 30 min was set to 100%. Results are given as mean
from n = 3 independent experiments ± SD.
HEK293

| | |
|---|---|
| IL-6 + | Phosphorylation in % |

| Sample | sIL-6R | IgG [μg/ml] | Mean | SD |
|---|---|---|---|---|
| Ctr. | − | | 0.0 | 0.0 |
| IL-6 + | + | | 100.0 | 0.0 |
| sIL-6R | | | | |
| 1H7 | + | 1 | 111.2 | 21.0 |
| IgG 07 | + | 1 | 2.8 | 1.5 |
| IgG 09 | + | 1 | 20.0 | 7.2 |
| IgG 12 | + | 1 | 4.0 | 5.2 |
| IgG 19 | + | 1 | 6.9 | 6.7 |
| IgG 20 | + | 1 | 0.2 | 0.3 |

Next, IL-6 classical- and combined IL-6 classical and trans-induced JAK/STAT signalling was analysed. The anti-Mesothelin control IgG antibody (11H7) has no effect on IL-6-induced STAT3 phosphorylation in HEK293-IL-6R cells, regardless of stimulation with IL-6 alone (representing IL-6 classical signalling) (Table 22) or IL-6 and soluble IL-6R together (representing combined IL-6 classical and trans (mixed) signalling) (Table 23), indicating that no unspecific inhibition of STAT3 activation by IgG antibodies occurs in HEK293_IL-6R cells. The five anti-IL-6R IgG antibodies do not significantly reduce STAT3 phosphorylation induced by either IL-6 (stimulating IL-6 classical signalling) (Table 22) or IL-6 and soluble IL-6R (stimulating combined IL-6 classical and trans (mixed) signalling (IL-6 mixed signalling)) (Table 23), with the exception of IgG 09, which slightly reduces IL-6 classical signalling-induced STAT3 phosphorylation (Table 22). As described previously, the classical:trans signalling ratio (Table 24) and mixed:trans signalling ratio (Table 25) was calculated using the IL-6 classical signalling or combined IL-6 classical and trans (mixed) signalling data of respectively.

TABLE 22
Inhibition of IL-6 classical signalling-induced
JAK/STAT signalling by anti-IL-6R IgG antibodies.
STAT3 phosphorylation of HEK293_IL-6R cells stimulated
with 10 ng/ml IL-6 for 30 min was set to 100%. Results are
given as mean from n = 3 independent experiments ± SD.
HEK293_IL-6R

Phosphorylation in %

| Sample | IL-6 | IgG [μg/ml] | Mean | SD |
|---|---|---|---|---|
| Ctr. | − | | 0.0 | 0.0 |
| IL-6 | + | | 100.0 | 0.0 |
| 1H7 | + | 1 | 107.8 | 7.8 |
| IgG 07 | + | 1 | 86.2 | 24.5 |
| IgG 09 | + | 1 | 69.7 | 20.5 |
| IgG 12 | + | 1 | 97.8 | 28.2 |
| IgG 19 | + | 1 | 89.5 | 36.2 |
| IgG 20 | + | 1 | 86.9 | 16.6 |

TABLE 23
Inhibition of combined IL-6 classical and trans
signalling-induced JAK/STAT signalling by anti-IL-6R
IgG antibodies. STAT3 phosphorylation of HEK293_IL-6R
cells induced by 10 ng/ml IL-6 and 200 ng/ml soluble
IL-6R (sIL-6R) was set to 100%. Results are given as mean
from n = 3 independent experiments.
HEK293_IL-6R

| | |
|---|---|
| IL-6 + | Phosphorylation in % |

| Sample | sIL-6R | IgG [μg/ml] | Mean | SD |
|---|---|---|---|---|
| Ctr. | − | | 0.0 | 0.0 |
| IL-6 + | + | | 100.0 | 0.0 |
| sIL-6R | | | | |
| 1H7 | + | 1 | 101.6 | 13.9 |
| IgG 07 | + | 1 | 105.2 | 31.9 |
| IgG 09 | + | 1 | 102.4 | 33.7 |
| IgG 12 | + | 1 | 99.9 | 32.4 |
| IgG 19 | + | 1 | 98.4 | 26.9 |
| IgG 20 | + | 1 | 88.1 | 14.5 |

TABLE 24
JAK/STAT signalling classical:trans signalling ratio of
anti-IL-6R IgG antibodies. Mean residual cytokine-induced
STAT3 phosphorylation in the presence of the indicated IgG antibodies
taken from Table 21 (HEK293, measuring IL-6 trans signalling)
and Table 22 (HEK293_IL-6R, measuring IL-6 classical
signalling) respectively.

| | | | Classical:trans |
|---|---|---|---|
| | | Residual | signalling ratio |
| | Residual signal of | proliferation of | intracellular |
| IgG | Ba/F3_gp130_IL-6R | Ba/F3_gp130 | signalling |
| 07 | 86 | 3 | 28.7 |
| 09 | 70 | 20 | 3.5 |
| 12 | 98 | 4 | 24.5 |
| 19 | 90 | 7 | 12.9 |
| 20 | 87 | 0.2 | 435.0 |

TABLE 25
JAK/STAT signalling mixed:trans signalling ratio of anti-
IL-6R IgG antibodies. Mean residual cytokine-induced STAT3
phosphorylation in the presence of the indicated IgG antibodies
taken from Table 21 (HEK293, measuring IL-6 trans signalling) and
Table 23 (HEK293_IL-6R, measuring combined IL-6 classical
and trans (mixed) signalling) respectively.

| | | | Mixed:trans |
|---|---|---|---|
| | | Residual | signalling ratio |
| | Residual signal of | proliferation of | intracellular |
| IgG | Ba/F3_gp130_IL-6R | Ba/F3_gp130 | signalling |
| 07 | 105 | 3 | 35.0 |
| 09 | 102 | 20 | 5.1 |
| 12 | 100 | 4 | 25.0 |
| 19 | 98 | 7 | 14.0 |
| 20 | 88 | 0.2 | 440.0 |

In summary, the inventors describe five anti-IL-6R IgG antibodies (IgG antibodies 07, 09, 12, 19 and 20) that selectively inhibit human IL-6 trans-signalling and not IL-6 classical signalling (i.e. inhibit IL-6 trans signalling while maintaining IL-6 classical signalling). Specifically, the IgG antibodies selectively inhibit cell proliferation and intracellular signalling induced by IL-6 trans signalling.

### Example 3—Production and Functional Characterisation of the Anti-IL-6R Fab C20

### Materials and Methods

### Cell Proliferation Assay to Analyse Inhibition of IL-6 Trans Signalling by the Anti-IL-6R Fab C20

Assays analysing cytokine-induced (IL-6 and soluble IL-6R) cell proliferation mediated by the IL-6 trans signalling pathway, and cell proliferation induced by Hyper-IL-6 (Hy-IL-6) trans signalling, were performed as described in Example 2 (i.e. using Ba/F3_gp130 cells), however the aliquots of cells were incubated with increasing concentrations of anti-IL-6R Fab C20 (3.3 ng/ml, 33.3 ng/ml, 100 ng/ml, 167 ng/ml, 233 ng/ml, 333 ng/ml and 3300 ng/ml; these concentrations correspond to the same molarities of the anti-IL-6R IgG antibodies used in Example 2).

### Results

As the anti-IL-6R IgG C20 had the highest capacity to inhibit IL-6 trans signalling from the candidate antibodies, a purified Fab fragment of C20 was produced.

### Inhibition of IL-6 Trans Signalling-Induced Cell Proliferation by the Anti-IL-6R Fab C20

The ability of the anti-IL-6R Fab C20 to block IL-6 trans signalling-induced cell proliferation was analyzed by using the Ba/F3 cellular system.

Stimulation of Ba/F3_gp130 cells with both IL-6 and soluble IL-6R, or with Hy-IL-6, results in IL-6 trans signalling-induced proliferation. As seen for the five anti-IL-6R IgG antibodies, the anti-IL-6R Fab C20 reduces IL-6 trans-signaling induced proliferation mediated by IL-6 and soluble IL-6R dose dependently (FIG. 8, Table 26), with a comparable IC50 to the anti-IL-6R IgG C20 (Table 7). The anti-IL-6R Fab C20 also reduces IL-6 trans-signaling induced proliferation mediated by Hy-IL-6 (Table 26), with a comparable IC50 to the anti-IL-6R IgG C20 (Table 8).

TABLE 26
IC50 of inhibition of IL-6 trans signalling-induced
proliferation by the anti-IL-6R fab C20. IC50 values,
in ng/ml and nM, for the inhibition of proliferation of
Ba/F3_gp130 cells induced by 10 ng/ml IL-6 and 200 ng/ml
sIL-6R, or by 28 ng/ml Hy-IL-6, by increasing concentrations
of the anti-IL-6R Fab C20 (3.3 ng/ml, 33.3 ng/ml, 100 ng/ml,
167 ng/ml, 233 ng/ml, 333 ng/ml and 3300 ng/ml).
Ba/F3_gp130

IC50

| Sample | Cytokine | ng/ml | nM |
|---|---|---|---|
| Fab 20 | IL-6:sIL-6R | 159 | 3.2 |
| Fab 20 | Hy-IL-6 | 98 | 1.9 |

This result shows the anti-IL-6R Fab C20 inhibits IL-6 trans signalling-induced proliferation, and suggests Fab fragments of the five anti-IL-6R IgG antibodies (C07, C09, C12, C19 and C20) retain the ability to selectively inhibit IL-6 trans signalling.

1. An antibody comprising at least one, preferably two, antigen binding domains that binds to human IL-6 receptor, wherein said antibody inhibits IL-6 trans signalling through the human soluble IL-6 receptor while maintaining IL-6 classical signalling through the human membrane-bound IL-6 receptor, and wherein said antibody can bind to human soluble IL-6 receptor and human membrane-bound IL-6 receptor.

2. The antibody of claim 1, wherein said antigen binding domain comprises a heavy chain variable region that comprises three complementarity determining regions (CDRs) and a light chain variable region that comprises three CDRs,
I. wherein said heavy chain variable region comprises:
(i) a variable heavy (VH) CDR1 that comprises the amino acid sequence of GFTFSNYG (SEQ ID NO:77) or a sequence substantially homologous thereto, wherein said substantially homologous sequence is a sequence containing 1, 2 or 3 amino acid substitutions,
(ii) a VH CDR2 that comprises the amino acid sequence of INSNGGST (SEQ ID NO:78) or a sequence substantially homologous thereto, wherein said substantially homologous sequence is a sequence containing 1, 2 or 3 amino acid substitutions, and
(iii) a VH CDR3 that comprises the amino acid sequence of ARDGNFVSDY (SEQ ID NO:79) or a sequence substantially homologous thereto, wherein said substantially homologous sequence is a sequence containing 1, 2 or 3 amino acid substitutions; and/or
wherein said light chain variable region comprises:
(iv) a variable light (VL) CDR1 that comprises the amino acid sequence of ESVDSYGNRF (SEQ ID NO:80) or a sequence substantially homologous thereto, wherein said substantially homologous sequence is a sequence containing 1, 2 or 3 amino acid substitutions,
(v) a VL CDR2 that comprises the amino acid sequence of LAS (SEQ ID NO:81) or a sequence substantially homologous thereto, wherein said substantially homologous sequence is a sequence containing 1 amino acid substitution, and
(vi) a VL CDR3 that comprises the amino acid sequence of QQNNEDPYT (SEQ ID NO:82) or a sequence substantially homologous thereto, wherein said substantially homologous sequence is a sequence containing 1, 2 or 3 amino acid substitutions; or
II. wherein said heavy chain variable region comprises:
(i) a variable heavy (VH) CDR1 that comprises the amino acid sequence of GFTFSSYA (SEQ ID NO:5) or a sequence substantially homologous thereto, wherein said substantially homologous sequence is a sequence containing 1, 2 or 3 amino acid substitutions,
(ii) a VH CDR2 that comprises the amino acid sequence of INSNGGST (SEQ ID NO:6) or a sequence substantially homologous thereto, wherein said substantially homologous sequence is a sequence containing 1, 2 or 3 amino acid substitutions, and
(iii) a VH CDR3 that comprises the amino acid sequence of AREGYYTMDY (SEQ ID NO:7) or a sequence substantially homologous thereto, wherein said substantially homologous sequence is a sequence containing 1, 2 or 3 amino acid substitutions; and/or
wherein said light chain variable region comprises:
(iv) a variable light (VL) CDR1 that comprises the amino acid sequence of ESVDSYGNSF (SEQ ID NO:8) or a sequence substantially homologous thereto, wherein said substantially homologous sequence is a sequence containing 1, 2 or 3 amino acid substitutions,
(v) a VL CDR2 that comprises the amino acid sequence of LAS (SEQ ID NO:9) or a sequence substantially homologous thereto, wherein said substantially homologous sequence is a sequence containing 1 amino acid substitution, and
(vi) a VL CDR3 that comprises the amino acid sequence of QQNNEDPYT (SEQ ID NO:10) or a sequence substantially homologous thereto, wherein said substantially homologous sequence is a sequence containing 1, 2 or 3 amino acid substitutions; or
III. wherein said heavy chain variable region comprises:
(i) a variable heavy (VH) CDR1 that comprises the amino acid sequence of GFTFSNYG (SEQ ID NO:41) or a sequence substantially homologous thereto, wherein said substantially homologous sequence is a sequence containing 1, 2 or 3 amino acid substitutions,
(ii) a VH CDR2 that comprises the amino acid sequence of INSNGGST (SEQ ID NO:42) or a sequence substantially homologous thereto, wherein said substantially homologous sequence is a sequence containing 1, 2 or 3 amino acid substitutions, and
(iii) a VH CDR3 that comprises the amino acid sequence of TRDGNFVSDY (SEQ ID NO:43) or a sequence substantially homologous thereto, wherein said substantially homologous sequence is a sequence containing 1, 2 or 3 amino acid substitutions; and/or
wherein said light chain variable region comprises:
(iv) a variable light (VL) CDR1 that comprises the amino acid sequence of ESVDSYGNSF (SEQ ID NO:44) or a sequence substantially homologous thereto, wherein said substantially homologous sequence is a sequence containing 1, 2 or 3 amino acid substitutions,
(v) a VL CDR2 that comprises the amino acid sequence of LAS (SEQ ID NO:45) or a sequence substantially homologous thereto, wherein said substantially homologous sequence is a sequence containing 1 amino acid substitution, and
(vi) a VL CDR3 that comprises the amino acid sequence of QQNNEDPYT (SEQ ID NO:46) or a sequence substantially homologous thereto, wherein said substantially homologous sequence is a sequence containing 1, 2 or 3 amino acid substitutions; or
IV. wherein said heavy chain variable region comprises:
(i) a variable heavy (VH) CDR1 that comprises the amino acid sequence of GFTFSNYG (SEQ ID NO:59) or a sequence substantially homologous thereto, wherein said substantially homologous sequence is a sequence containing 1, 2 or 3 amino acid substitutions,
(ii) a VH CDR2 that comprises the amino acid sequence of MNSKGGST (SEQ ID NO:60) or a sequence substantially homologous thereto, wherein said substantially homologous sequence is a sequence containing 1, 2 or 3 amino acid substitutions, and
(iii) a VH CDR3 that comprises the amino acid sequence of ARDGYYTMDY (SEQ ID NO:61) or a sequence substantially homologous thereto, wherein said substantially homologous sequence is a sequence containing 1, 2 or 3 amino acid substitutions; and/or
wherein said light chain variable region comprises:
(iv) a variable light (VL) CDR1 that comprises the amino acid sequence of KSVDSFGNSF (SEQ ID NO:62) or a sequence substantially homologous thereto, wherein said substantially homologous sequence is a sequence containing 1, 2 or 3 amino acid substitutions,
(v) a VL CDR2 that comprises the amino acid sequence of LAS (SEQ ID NO:63) or a sequence substantially homologous thereto, wherein said substantially homologous sequence is a sequence containing 1 amino acid substitution, and
(vi) a VL CDR3 that comprises the amino acid sequence of QQNNEDPYT (SEQ ID NO:64) or a sequence substantially homologous thereto, wherein said substantially homologous sequence is a sequence containing 1, 2 or 3 amino acid substitutions; or
V. wherein said heavy chain variable region comprises:
(i) a variable heavy (VH) CDR1 that comprises the amino acid sequence of GFTFSNYG (SEQ ID NO:23) or a sequence substantially homologous thereto, wherein said substantially homologous sequence is a sequence containing 1, 2 or 3 amino acid substitutions,
(ii) a VH CDR2 that comprises the amino acid sequence of INSNGGST (SEQ ID NO:24) or a sequence substantially homologous thereto, wherein said substantially homologous sequence is a sequence containing 1, 2 or 3 amino acid substitutions, and
(iii) a VH CDR3 that comprises the amino acid sequence of ARDGNYVSDY (SEQ ID NO:25) or a sequence substantially homologous thereto, wherein said substantially homologous sequence is a sequence containing 1, 2 or 3 amino acid substitutions; and/or
wherein said light chain variable region comprises:
(iv) a variable light (VL) CDR1 that comprises the amino acid sequence of ESVDSYGNSF (SEQ ID NO:26) or a sequence substantially homologous thereto, wherein said substantially homologous sequence is a sequence containing 1, 2 or 3 amino acid substitutions,
(v) a VL CDR2 that comprises the amino acid sequence of LAS (SEQ ID NO:27) or a sequence substantially homologous thereto, wherein said substantially homologous sequence is a sequence containing 1 amino acid substitution, and
(vi) a VL CDR3 that comprises the amino acid sequence of QQNNEDPWT (SEQ ID NO:28) or a sequence substantially homologous thereto, wherein said substantially homologous sequence is a sequence containing 1, 2 or 3 amino acid substitutions.

3. The antibody of claim 1 or 2, wherein said antigen binding domain comprises a heavy chain variable region that comprises three complementarity determining regions (CDRs) and a light chain variable region that comprises three CDRs, wherein said heavy chain region comprises:
a) a variable heavy (VH) CDR1 that comprises the amino acid sequence of GFTFSX₆YX₇, wherein X₆ and X₈ is any amino acid (SEQ ID NO:91), wherein X₃ is preferably N or S, and/or X₈ is preferably G or A;
b) a VH CDR2 that comprises the amino acid sequence of X₁NSX₄GGST, wherein X₁ and X₄ is any amino acid (SEQ ID NO:94), wherein X₁ is preferably I or M, and/or X₄ is preferably N or K; and
c) a VH CDR3 that comprises the amino acid sequence X₁RX₃GX₅X₆X₇X₈DY, wherein X₁, X₃, X₅, X₆, X₇, and X₃ is any amino acid (SEQ ID NO:97), wherein X₁ is preferably A or T, X₃ is preferably D or E, X₅ is preferably N or Y, X₆ is preferably Y or F, X₇ is preferably V or T, and/or X₃ is preferably S or M,
preferably wherein the amino acid sequence comprises ARX₃GYYTMDY, wherein X₃ is any amino acid (SEQ ID NO:100), preferably E or D, or wherein the amino acid sequence comprises X₁RDGNX₆VSDY, wherein X₁ and X₆ is any amino acid (SEQ ID NO:103), wherein X₁ is preferably A or T, and/or X₆ is preferably F or Y; and/or
wherein said light chain variable region comprises:
d) a variable light (VL) CDR1 that comprises the amino acid sequence of X₁SVDSX₆GNX₉F, wherein X₁, X₆, and X₉ is any amino acid (SEQ ID NO:106), wherein X₁ is preferably E or K, X₆ is preferably Y or F, and/or X₉ is preferably S or R; and
e) a VL CDR2 that comprises the amino acid sequence of LAS (SEQ ID NO:9), or a sequence substantially homologous thereto, wherein said substantially homologous sequence is a sequence containing 1 amino acid substitution; and
f) a VL CDR3 that comprises the amino acid sequence of QQNNEDPX₈T, wherein X₈ is any amino acid (SEQ ID NO:109), preferably Y or W.

4. The antibody of any one of claims 1 to 3, wherein said antigen binding domain comprises a heavy chain variable region that comprises three CDRs and a light chain variable region that comprises three CDRs,
I. wherein said heavy chain variable region comprises the amino acid sequence of SEQ ID NO:75, or a sequence having at least 80% sequence identity thereto, and/or
wherein said light chain variable region comprises the amino acid sequence of SEQ ID NO:76, or a sequence having at least 80% sequence identity thereto; or
II. wherein said heavy chain variable region comprises the amino acid sequence of SEQ ID NO:3, or a sequence having at least 80% sequence identity thereto, and/or
wherein said light chain variable region comprises the amino acid sequence of SEQ ID NO:4, or a sequence having at least 80% sequence identity thereto; or
III. wherein said heavy chain variable region comprises the amino acid sequence of SEQ ID NO:39, or a sequence having at least 80% sequence identity thereto, and/or
wherein said light chain variable region comprises the amino acid sequence of SEQ ID NO:40, or a sequence having at least 80% sequence identity thereto; or
IV. wherein said heavy chain variable region comprises the amino acid sequence of SEQ ID NO:57, or a sequence having at least 80% sequence identity thereto, and/or
wherein said light chain variable region comprises the amino acid sequence of SEQ ID NO:58, or a sequence having at least 80% sequence identity thereto; or
V. wherein said heavy chain variable region comprises the amino acid sequence of SEQ ID NO:21, or a sequence having at least 80% sequence identity thereto, and/or
wherein said light chain variable region comprises the amino acid sequence of SEQ ID NO:22, or a sequence having at least 80% sequence identity thereto.

5. The antibody of any one of claims 1 to 4, wherein said antibody is a monoclonal antibody.

6. The antibody of any one of claims 1 to 5, wherein said antibody is a full-length antibody.

7. The antibody of any one of claims 1 to 5, wherein said antibody is an antigen binding fragment of an antibody, preferably a Fab fragment.

8. The antibody of any one of claims 1 to 7, wherein said antibody is PEGylated.

9. An antibody that binds to the same epitope on human IL-6 receptor as the antibody of any one of claims 2 to 4.

10. An antigen binding protein comprising an antibody or antigen binding domain as defined in any one of claims 1 to 9.

11. A composition comprising an antibody of any one of claims 1 to 9, or an antigen binding protein of claim 10, and a diluent, carrier or excipient, preferably a pharmaceutically acceptable diluent, carrier or excipient.

12. A nucleic acid molecule comprising a nucleotide sequence that encodes an antibody of any one of claims 1 to 9 or an antigen binding protein of claim 10, or a set of nucleic acid molecules each comprising a nucleotide sequence, wherein said set of nucleic acid molecules together encode an antibody of any one of claims 1 to 9 or an antigen binding protein of claim 10.

13. One or more expression vectors comprising one or more of the nucleic acid molecules of claim 12.

14. One or most host cells or viruses comprising said expression vectors of claim 13, or said nucleic acid molecules of claim 12, or expressing the antibody or antigen binding protein of any one of claims 1 to 10.

15. A method of producing the antibody or antigen binding protein of any one of claims 1 to 10, comprising the steps of:
(i) culturing a host cell comprising one or more nucleic acid molecules as defined in claim 12 or one or more expression vectors as defined in claim 13, under conditions suitable for the expression of the encoded antibody or antigen binding protein; and
(ii) isolating or obtaining the antibody or antigen binding protein from the host cell or from the growth medium/supernatant.

16. An antibody, antigen binding protein, or composition as defined in any one of claims 1 to 11 for use in therapy.

17. An antibody, antigen binding protein, or composition as defined in any one of claims 1 to 11 for use in treating a disease or disorder, wherein the disease or disorder is an inflammatory disease, a CNS disorder or cancer, preferably wherein said disease or disorder is one or more of: cancer, rheumatoid arthritis, Systemic juvenile idiopathic arthritis, Castleman disease, cytokine release syndrome, multiple sclerosis, anaemia, irritable bowel syndrome (IBS), inflammatory bowel disease (IBD), pancreatitis, acute lung injury (ALI), depression, Alzheimer's disease, sepsis, or Covid-19 or Covid-19 complications.

18. A method of treating a disease or disorder, said method comprising administering to a patient in need thereof a therapeutically effective amount of an antibody, antigen binding protein, or composition as defined in any one of claims 1 to 11, wherein the disease or disorder is an inflammatory disease, a CNS disorder or cancer, preferably wherein said disease or disorder is one or more of: cancer, rheumatoid arthritis, Systemic juvenile idiopathic arthritis, Castleman disease, cytokine release syndrome, multiple sclerosis, anaemia, irritable bowel syndrome (IBS), inflammatory bowel disease (IBD), pancreatitis, acute lung injury (ALI), depression, Alzheimer's disease, sepsis, or Covid-19 or Covid-19 complications.

19. Use of an antibody, antigen binding protein, or composition as defined in any one of claims 1 to 11 in the manufacture of a medicament for use in therapy.

20. The use according to claim 19, wherein said therapy is the treatment of an inflammatory disease, a CNS disorder or cancer, preferably one or more of: cancer, rheumatoid arthritis, Systemic juvenile idiopathic arthritis, Castleman disease, cytokine release syndrome, multiple sclerosis, anaemia, irritable bowel syndrome (IBS), inflammatory bowel disease (IBD), pancreatitis, acute lung injury (ALI), depression, Alzheimer's disease, sepsis, or Covid-19 or Covid-19 complications.


SEQUENCE LISTING (116 sequences)

SEQ ID NO: 1 - DNA, 351 bp, synthetic construct
  source 1..351: mol_type = other DNA
gaggtgaagctggtggaatctgggggaggcttagtgcagcctggagggtccctgaaactctcctgtgcagcctctggattcactttcagtagctatgccatgtcttgggttcgccagactgcagacaagaggctggagttggtcgcaaacattaatagtaatggtggtagtacctattatccagacagtgtgaagggccgattcaccatctccagagacaatgccaagaacaccctgttcctgcaaatgagcagtctgaagtctgaggacacagccatgtattactgtgcaagagaaggttactacactatggactactggggtcaaggaacctcagtcaccgtctcctca

SEQ ID NO: 2 - DNA, 333 bp, synthetic construct
  source 1..333: mol_type = other DNA
gacattgtgctgacccaatctccagcttctttggctgtgtctccagggcagagggccaccatatcctgcagagccagtgaaagtgttgatagttatggcaatagttttatgaactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtacacgttcggaggggggaccaagctggaaataaaa

SEQ ID NO: 3 - protein, 117 aa, synthetic construct
EVKLVESGGGLVQPGGSLKLSCAASGFTFSSYAMSWVRQTADKRLELVANINSNGGSTYYPDSVKGRFTISRDNAKNTLFLQMSSLKSEDTAMYYCAREGYYTMDYWGQGTSVTVSS

SEQ ID NO: 4 - protein, 111 aa, synthetic construct
DIVLTQSPASLAVSPGQRATISCRASESVDSYGNSFMNWYQQKPGQPPKLLIYLASNLESGVPARFSGSGSRTDFTLTIDPVEADDAATYYCQQNNEDPYTFGGGTKLEIK

SEQ ID NO: 5 - protein, 8 aa, synthetic construct
GFTFSSYA

SEQ ID NO: 6 - protein, 8 aa, synthetic construct
INSNGGST

SEQ ID NO: 7 - protein, 10 aa, synthetic construct
AREGYYTMDY

SEQ ID NO: 8 - protein, 10 aa, synthetic construct
ESVDSYGNSF

SEQ ID NO: 9 - skipped

SEQ ID NO: 10 - protein, 9 aa, synthetic construct
QQNNEDPYT

SEQ ID NO: 11 - protein, 25 aa, synthetic construct
EVKLVESGGGLVQPGGSLKLSCAAS

SEQ ID NO: 12 - protein, 17 aa, synthetic construct
MSWVRQTADKRLELVAN

SEQ ID NO: 13 - protein, 38 aa, synthetic construct
YYPDSVKGRFTISRDNAKNTLFLQMSSLKSEDTAMYYC

SEQ ID NO: 14 - protein, 11 aa, synthetic construct
WGQGTSVTVSS

SEQ ID NO: 15 - protein, 26 aa, synthetic construct
DIVLTQSPASLAVSPGQRATISCRAS

SEQ ID NO: 16 - protein, 17 aa, synthetic construct
MNWYQQKPGQPPKLLIY

SEQ ID NO: 17 - protein, 36 aa, synthetic construct
NLESGVPARFSGSGSRTDFTLTIDPVEADDAATYYC

SEQ ID NO: 18 - protein, 10 aa, synthetic construct
FGGGTKLEIK

SEQ ID NO: 19 - DNA, 351 bp, synthetic construct
  source 1..351: mol_type = other DNA
gaggtgcagcttgttgagtctgggggagacttagtgcagcctggagggtccctgaaactctcctgtgcagcctctggattcactttcagtaactatggcatgtcttgggttcgccagactccagacaagaggctggaattggtcgcagccattaatagtaatggtggtagcacctattatccagacagtgtgaagggccgattcaccatctccagagacaatgccaagaacaccctgtacctgcaaatgagcagtctgaagtctgaggacacagccatgtattactgtgcaagggacggtaactacgtatctgactactggggccaaggcaccactctcacagtctcctca

SEQ ID NO: 20 - DNA, 333 bp, synthetic construct
  source 1..333: mol_type = other DNA
gacattgtgctgacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaa

SEQ ID NO: 21 - protein, 117 aa, synthetic construct
EVQLVESGGDLVQPGGSLKLSCAASGFTFSNYGMSWVRQTPDKRLELVAAINSNGGSTYYPDSVKGRFTISRDNAKNTLYLQMSSLKSEDTAMYYCARDGNYVSDYWGQGTTLTVSS

SEQ ID NO: 22 - protein, 111 aa, synthetic construct
DIVLTQSPASLAVSLGQRATISCRASESVDSYGNSFMHWYQQKPGQPPKLLIYLASNLESGVPARFSGSGSRTDFTLTIDPVEADDAATYYCQQNNEDPWTFGGGTKLEIK

SEQ ID NO: 23 - protein, 8 aa, synthetic construct
GFTFSNYG

SEQ ID NO: 24 - protein, 8 aa, synthetic construct
INSNGGST

SEQ ID NO: 25 - protein, 10 aa, synthetic construct
ARDGNYVSDY

SEQ ID NO: 26 - protein, 10 aa, synthetic construct
ESVDSYGNSF

SEQ ID NO: 27 - skipped

SEQ ID NO: 28 - protein, 9 aa, synthetic construct
QQNNEDPWT

SEQ ID NO: 29 - protein, 25 aa, synthetic construct
EVQLVESGGDLVQPGGSLKLSCAAS

SEQ ID NO: 30 - protein, 17 aa, synthetic construct
MSWVRQTPDKRLELVAA

SEQ ID NO: 31 - protein, 38 aa, synthetic construct
YYPDSVKGRFTISRDNAKNTLYLQMSSLKSEDTAMYYC

SEQ ID NO: 32 - protein, 11 aa, synthetic construct
WGQGTTLTVSS

SEQ ID NO: 33 - protein, 26 aa, synthetic construct
DIVLTQSPASLAVSLGQRATISCRAS

SEQ ID NO: 34 - protein, 17 aa, synthetic construct
MHWYQQKPGQPPKLLIY

SEQ ID NO: 35 - protein, 36 aa, synthetic construct; identical to SEQ ID NO: 17

SEQ ID NO: 36 - protein, 10 aa, synthetic construct
FGGGTKLEIK

SEQ ID NO: 37 - DNA, 351 bp, synthetic construct
  source 1..351: mol_type = other DNA
gaggtgaagctgatggaatctggaggaggcttagtgcagcctggagggtccctgaaactctcctgtgcagcctctggattcactttcagtaactatggcatgtcttgggttcgccagactccagacaagaggctggaatgggtcgcaaacattaatagtaatggtgggagcacctattatccagacagtgtgaagggccgattcaccatctccagagacaatgcccagaacaccctgtatctgcaaatgagcagtctgaagtctgaggacacagccatgtattactgtacaagagacggtaacttcgtatctgactactggggccaaggcaccactctcacagtctcctca

SEQ ID NO: 38 - DNA, 333 bp, synthetic construct; identical to SEQ ID NO: 2
  source 1..333: mol_type = other DNA

SEQ ID NO: 39 - protein, 117 aa, synthetic construct
EVKLMESGGGLVQPGGSLKLSCAASGFTFSNYGMSWVRQTPDKRLEWVANINSNGGSTYYPDSVKGRFTISRDNAQNTLYLQMSSLKSEDTAMYYCTRDGNFVSDYWGQGTTLTVSS

SEQ ID NO: 40 - protein, 111 aa, synthetic construct; identical to SEQ ID NO: 4

SEQ ID NO: 41 - protein, 8 aa, synthetic construct
GFTFSNYG

SEQ ID NO: 42 - protein, 8 aa, synthetic construct
INSNGGST

SEQ ID NO: 43 - protein, 10 aa, synthetic construct
TRDGNFVSDY

SEQ ID NO: 44 - protein, 10 aa, synthetic construct
ESVDSYGNSF

SEQ ID NO: 45 - skipped

SEQ ID NO: 46 - protein, 9 aa, synthetic construct
QQNNEDPYT

SEQ ID NO: 47 - protein, 25 aa, synthetic construct
EVKLMESGGGLVQPGGSLKLSCAAS

SEQ ID NO: 48 - protein, 17 aa, synthetic construct
MSWVRQTPDKRLEWVAN

SEQ ID NO: 49 - protein, 38 aa, synthetic construct
YYPDSVKGRFTISRDNAQNTLYLQMSSLKSEDTAMYYC

SEQ ID NO: 50 - protein, 11 aa, synthetic construct
WGQGTTLTVSS

SEQ ID NO: 51 - protein, 26 aa, synthetic construct
DIVLTQSPASLAVSPGQRATISCRAS

SEQ ID NO: 52 - protein, 17 aa, synthetic construct
MNWYQQKPGQPPKLLIY

SEQ ID NO: 53 - protein, 36 aa, synthetic construct; identical to SEQ ID NO: 17

SEQ ID NO: 54 - protein, 10 aa, synthetic construct
FGGGTKLEIK

SEQ ID NO: 55 - DNA, 351 bp, synthetic construct
  source 1..351: mol_type = other DNA
gaggtgaagctgatggaatctgggggaggcttagtgcagcctggagggtccctgaaattgtcctgtgcagcctctggattcactttcagtaactatggcatgtcttgggttcgccagactccagacaagaggctggagttggtcgcaattatgaatagtaaaggtggtagcacctattatccagacagtgtgatgggccgattcaccatctccagagacaatgccaaaaacaccctgtacctgcaaatgagcagtctgaagtctgaggacacagccatgtattactgtgcaagagatggttactacactatggactactggggtcaaggaacctcagtcaccgtctcctca

SEQ ID NO: 56 - DNA, 333 bp, synthetic construct
  source 1..333: mol_type = other DNA
gacattgtgctgacccaatctccagcttctttggctgtgtctctagggcagagggccaccatatcctgcagagccagtaaaagtgttgatagttttggcaatagttttatgcactggtaccagcagaaaccaggacagtcacccagactcctcatcttccttgcatccaacctagaatctggggtccctgccagattcagtggcagtgggtctaggacagacttcaccctcaccattgatcctgtggaggctgatgatgctgcaacctattactgtcagcaaaataatgaggatccatacacgttcggaggggggaccaagctggaaataaaa

SEQ ID NO: 57 - protein, 117 aa, synthetic construct
EVKLMESGGGLVQPGGSLKLSCAASGFTFSNYGMSWVRQTPDKRLELVAIMNSKGGSTYYPDSVMGRFTISRDNAKNTLYLQMSSLKSEDTAMYYCARDGYYTMDYWGQGTSVTVSS

SEQ ID NO: 58 - protein, 111 aa, synthetic construct
DIVLTQSPASLAVSLGQRATISCRASKSVDSFGNSFMHWYQQKPGQSPRLLIFLASNLESGVPARFSGSGSRTDFTLTIDPVEADDAATYYCQQNNEDPYTFGGGTKLEIK

SEQ ID NO: 59 - protein, 8 aa, synthetic construct
GFTFSNYG

SEQ ID NO: 60 - protein, 8 aa, synthetic construct
MNSKGGST

SEQ ID NO: 61 - protein, 10 aa, synthetic construct
ARDGYYTMDY

SEQ ID NO: 62 - protein, 10 aa, synthetic construct
KSVDSFGNSF

SEQ ID NO: 63 - skipped

SEQ ID NO: 64 - protein, 9 aa, synthetic construct
QQNNEDPYT

SEQ ID NO: 65 - protein, 25 aa, synthetic construct
EVKLMESGGGLVQPGGSLKLSCAAS

SEQ ID NO: 66 - protein, 17 aa, synthetic construct
MSWVRQTPDKRLELVAI

SEQ ID NO: 67 - protein, 38 aa, synthetic construct
YYPDSVMGRFTISRDNAKNTLYLQMSSLKSEDTAMYYC

SEQ ID NO: 68 - protein, 11 aa, synthetic construct
WGQGTSVTVSS

SEQ ID NO: 69 - protein, 26 aa, synthetic construct
DIVLTQSPASLAVSLGQRATISCRAS

SEQ ID NO: 70 - protein, 17 aa, synthetic construct
MHWYQQKPGQSPRLLIF

SEQ ID NO: 71 - protein, 36 aa, synthetic construct; identical to SEQ ID NO: 17

SEQ ID NO: 72 - protein, 10 aa, synthetic construct
FGGGTKLEIK

SEQ ID NO: 73 - DNA, 351 bp, synthetic construct
  source 1..351: mol_type = other DNA
gaggtgaagctgatggaatctgggggaggcttagtgcagcctggagggtccctgaaactctcctgtgcagcctctggattcactttcagtaactatggcatgtcttgggttcgccagactccagacaagaggctggagttggtcgcaatcattaatagtaatggtggtagcacctattatccagacagtgtgaagggccgattcaccatctccagagacaatgccaagaacaccctgtacctgcaaatgagcagtctgaagtctgaggacacagccatatattactgtgcaagagacggtaacttcgtatctgactactggggccaaggcaccactctcacagtctcctca

SEQ ID NO: 74 - DNA, 333 bp, synthetic construct
  source 1..333: mol_type = other DNA
gacattgtgctgacccaatctccagcttctttggctgtgtctctagggcagagggccaccatatcctgcagagccagtgaaagtgttgatagttatggcaataggtttttgaactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtacacgttcggaggggggaccaagctggaaataaaa

SEQ ID NO: 75 - protein, 117 aa, synthetic construct
EVKLMESGGGLVQPGGSLKLSCAASGFTFSNYGMSWVRQTPDKRLELVAIINSNGGSTYYPDSVKGRFTISRDNAKNTLYLQMSSLKSEDTAIYYCARDGNFVSDYWGQGTTLTVSS

SEQ ID NO: 76 - protein, 111 aa, synthetic construct
DIVLTQSPASLAVSLGQRATISCRASESVDSYGNRFLNWYQQKPGQPPKLLIYLASNLESGVPARFSGSGSRTDFTLTIDPVEADDAATYYCQQNNEDPYTFGGGTKLEIK

SEQ ID NO: 77 - protein, 8 aa, synthetic construct
GFTFSNYG

SEQ ID NO: 78 - protein, 8 aa, synthetic construct
INSNGGST

SEQ ID NO: 79 - protein, 10 aa, synthetic construct
ARDGNFVSDY

SEQ ID NO: 80 - protein, 10 aa, synthetic construct
ESVDSYGNRF

SEQ ID NO: 81 - skipped

SEQ ID NO: 82 - protein, 9 aa, synthetic construct
QQNNEDPYT

SEQ ID NO: 83 - protein, 25 aa, synthetic construct
EVKLMESGGGLVQPGGSLKLSCAAS

SEQ ID NO: 84 - protein, 17 aa, synthetic construct
MSWVRQTPDKRLELVAI

SEQ ID NO: 85 - protein, 38 aa, synthetic construct
YYPDSVKGRFTISRDNAKNTLYLQMSSLKSEDTAIYYC

SEQ ID NO: 86 - protein, 11 aa, synthetic construct
WGQGTTLTVSS

SEQ ID NO: 87 - protein, 26 aa, synthetic construct
DIVLTQSPASLAVSLGQRATISCRAS

SEQ ID NO: 88 - protein, 17 aa, synthetic construct
LNWYQQKPGQPPKLLIY

SEQ ID NO: 89 - protein, 36 aa, synthetic construct; identical to SEQ ID NO: 17

SEQ ID NO: 90 - protein, 10 aa, synthetic construct
FGGGTKLEIK

SEQ ID NO: 91 - protein, 8 aa, synthetic construct
GFTFSXYX

SEQ ID NO: 92 - protein, 8 aa, synthetic construct
  VARIANT 6: note = N or S
  VARIANT 8: note = G or A
GFTFSXYX

SEQ ID NO: 93 - protein, 8 aa, synthetic construct
  VARIANT 6: note = N or S
  VARIANT 8: note = G or A
GFTFSXYX

SEQ ID NO: 94 - protein, 8 aa, synthetic construct
XNSXGGST

SEQ ID NO: 95 - protein, 8 aa, synthetic construct
  VARIANT 1: note = I or M
  VARIANT 4: note = N or K
XNSXGGST

SEQ ID NO: 96 - protein, 8 aa, synthetic construct
  VARIANT 1: note = I or M
  VARIANT 4: note = N or K
XNSXGGST

SEQ ID NO: 97 - protein, 10 aa, synthetic construct
XRXGXXXXDY

SEQ ID NO: 98 - protein, 10 aa, synthetic construct
  VARIANT 1: note = A or T
  VARIANT 3: note = D or E
  VARIANT 5: note = N or Y
  VARIANT 6: note = Y or F
  VARIANT 7: note = V or T
  VARIANT 8: note = S or M
XRXGXXXXDY

SEQ ID NO: 99 - protein, 10 aa, synthetic construct
  VARIANT 1: note = A or T
  VARIANT 3: note = D or E
  VARIANT 5: note = N or Y
  VARIANT 6: note = Y or F
  VARIANT 7: note = V or T
  VARIANT 8: note = S or M
XRXGXXXXDY

SEQ ID NO: 100 - protein, 10 aa, synthetic construct
ARXGYYTMDY

SEQ ID NO: 101 - protein, 10 aa, synthetic construct
  VARIANT 3: note = E or D
ARXGYYTMDY

SEQ ID NO: 102 - protein, 10 aa, synthetic construct
  VARIANT 3: note = E or D
ARXGYYTMDY

SEQ ID NO: 103 - protein, 10 aa, synthetic construct
XRDGNXVSDY

SEQ ID NO: 104 - protein, 10 aa, synthetic construct
  VARIANT 1: note = A or T
  VARIANT 6: note = F or Y
XRDGNXVSDY

SEQ ID NO: 105 - protein, 10 aa, synthetic construct
  VARIANT 1: note = A or T
  VARIANT 6: note = F or Y
XRDGNXVSDY

SEQ ID NO: 106 - protein, 10 aa, synthetic construct
XSVDSXGNXF

SEQ ID NO: 107 - protein, 10 aa, synthetic construct
  VARIANT 1: note = E or K
  VARIANT 6: note = Y or F
  VARIANT 9: note = S or R
XSVDSXGNXF

SEQ ID NO: 108 - protein, 10 aa, synthetic construct
  VARIANT 1: note = E or K
  VARIANT 6: note = Y or F
  VARIANT 9: note = S or R
XSVDSXGNXF

SEQ ID NO: 109 - protein, 9 aa, synthetic construct
QQNNEDPXT

SEQ ID NO: 110 - protein, 9 aa, synthetic construct
  VARIANT 8: note = Y or W
QQNNEDPXT

SEQ ID NO: 111 - protein, 9 aa, synthetic construct
  VARIANT 8: note = Y or W
QQNNEDPXT

SEQ ID NO: 112 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 113 - protein, 357 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQ

SEQ ID NO: 114 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 115 - protein, 460 aa, Mus musculus
MLTVGCTLLVALLAAPAVALVLGSCRALEVANGTVTSLPGATVTLICPGKEAAGNVTIHWVYSGSQNREWTTTGNTLVLRDVQLSDTGDYLCSLNDHLVGTVPLLVDVPPEEPKLSCFRKNPLVNAICEWRPSSTPSPTTKAVLFAKKINTTNGKSDFQVPCQYSQQLKSFSCQVEILEGDKVYHIVSLCVANSVGSKSSHNEAFHSLKMVQPDPPANLVVSAIPGRPRWLKVSWQHPETWDPSYYLLQFQLRYRPVWSKEFTVLLLPVAQYQCVIHDALRGVKHVVQVRGKEELDLGQWSEWSPEVTGTPWIAEPRTTPAGILWNPTQVSVEDSANHEDQYESSTEATSVLAPVQESSSMSLPTFLVAGGSLAFGLLLCVFIILRLKQKWKSEAEKESKTTSPPPPPYSLGPLKPTFLLVPLLTPHSSGSDNTVNHSCLGVRDAQSPYDNSNRDYLFPR

SEQ ID NO: 116 - protein, 328 aa, Mus musculus
LVLGSCRALEVANGTVTSLPGATVTLICPGKEAAGNVTIHWVYSGSQNREWTTTGNTLVLRDVQLSDTGDYLCSLNDHLVGTVPLLVDVPPEEPKLSCFRKNPLVNAICEWRPSSTPSPTTKAVLFAKKINTTNGKSDFQVPCQYSQQLKSFSCQVEILEGDKVYHIVSLCVANSVGSKSSHNEAFHSLKMVQPDPPANLVVSAIPGRPRWLKVSWQHPETWDPSYYLLQFQLRYRPVWSKEFTVLLLPVAQYQCVIHDALRGVKHVVQVRGKEELDLGQWSEWSPEVTGTPWIAEPRTTPAGILWNPTQVSVEDSANHEDQYESSTE